(19) United States
(12) Patent Application Publication
(10) Pub. No.: US 2026/0297166 A1
(43) Pub. Date: Oct. 1, 2026

(54) ASYMMETRIC CIRCULAR TANDEM REPEAT PROTEINS

(71) Applicant: Seattle Children's Hospital d/b/a Seattle Children's Research Institute, Seattle, WA (US)

(72) Inventors: Jason Price, Seattle, WA (US); Raymond Ruff, Seattle, WA (US); Bianka Haro, Seattle, WA (US); James M. Olson, Seattle, WA (US); Sinduja Marx, Seattle, WA (US); Eric Nealy, Seattle, WA (US)

(73) Assignee: Seattle Children's Hospital d/b/a Seattle Children's Research Institute, Seattle, WA (US)

(21) Appl. No.: 19/478,671

(22) PCT Filed: May 15, 2024
(86) PCT No.: PCT/US2024/029426

§ 371 Date: Oct. 24, 2025

Related U.S. Application Data
(60) Provisional application No. 63/466,622, filed on May 15, 2023.

(51) Int. Cl. C07K 14/715; A61K 39/00; A61K 39/145; C07K 14/705; C07K 16/108

(52) U.S. Cl. CPC C07K 14/7155; A61K 39/145; C07K 14/70532; C07K 16/108; A61K 2039/6031; A61K 2039/70

(57) ABSTRACT

In certain embodiments, the present invention provides protomers and asymmetrical circular tandem repeat proteins (cTRPs). Asymmetric cTRPs are provided as assemblage of two or more protomers having amino acid substitutions with cysteine or with non-canonical amino acids at different amino acid residue positions on respective terminal α-helical structural units, thereby facilitating disulfide bonding or click conjugation in between adjacent protomers so as to form hetero-oligomer cTRPs with interface bonding/conjugation. Preferably cargo molecules are operably linked to the protomers, so as to form an asymmetrical cTRPs with different cargo molecules.

### CROSS-REFERENCE TO RELATED APPLICATIONS

This application includes a claim of priority under 35 U.S.C. § 119 (e) to U.S. provisional patent application No. 63/466,622, filed May 15, 2023, the entirety of which is hereby incorporated by reference.

### REFERENCE TO SEQUENCE LISTING

This application contains a Sequence Listing submitted as a computer readable form named “067505_000015WOPT_SequenceListing”, having a size in bytes of 502, 184 bytes, and created on May 14, 2024. The information contained in this computer readable form is hereby incorporated by reference in its entirety.

### FIELD OF INVENTION

This invention relates to artificially designed circular tandem repeat proteins (cTRPs), specifically asymmetrical cTRPs with disulfide or otherwise conjugated linkages, and their uses as scaffolds for geometrically precise presentation of protein and peptide epitopes related to immunology or other biochemical applications.

### BACKGROUND

Proteins containing internal repetitions are found in nature. Such protein repeats possess regular secondary structures and form multi-repeat assemblies in three dimensions of diverse sizes and functions. Repeat proteins are formed by the repeating of modular units of protein sub-structures. The structure of the repeat proteins as a whole is dictated by the internal geometry of the protein and the local packing of the repeat modular units. These features are generated by underlying patterns of amino acid sequences that are repetitive.

Repeat proteins function in nature as macromolecular binding and scaffolding domains, enzymes, and building blocks for the assembly of fibrous materials. The structure and identity of these repeat proteins are highly diverse, ranging from extended, super-helical folds that bind peptide, DNA, and RNA partners, to closed and compact conformations with internal cavities suitable for small molecule binding and catalysis.

Tandem repeat protein (“TRPs”) are proteins containing multiple repeated peptide sequences. TRPs form highly similar folded topologies that self-associate to form closed circular or open linear protein scaffolds. Naturally occurring TRPs can bind to a variety of protein and nucleic acid partners. TRPs are straightforward to redesign and often display several favorable biophysical properties, including high thermal stability and solubility, and ease of expression. TRPs have been used as scaffolds to host various protein domains.

Non-naturally occurring circular tandem repeat proteins (cTRPs) have been engineered to be protein scaffolds that are generally based on anti-parallel two-helix bundles. They contain repeated protein sequences and structural motifs and form closed circular structures. cTRPs can display significant stability and solubility, a wide range of sizes, and are useful as protein display particles for biotechnology applications. Symmetrical cTRPs are discussed in PCT Publication WO 2017/096236 A1 or U.S. Pat. No. 10,597,466, and WO 2021/003413 A1 or US20220411472, all of which are incorporated by reference in their entirety herein. Symmetric protein-based nanoparticles that display multiple protein domains can exhibit enhanced functional properties due to increased avidity and improved solution behavior and stability. The creation and characterization of a computationally designed cTRP composed of 24 identical repeated motifs that can display a variety of functional protein domains (“cargo”) at defined positions around its periphery has been discussed. Such designed proteins were expressed at very high levels, were extremely thermostable and soluble, and formed structures that corresponded closely to their original designs.

Many proteins have a natural tendency to self-associate into homo-oligomeric protein complexes or assemblies, which are composed of two or more identical subunits. The vast majority of such assemblies possess internal symmetry. Symmetry is an inherent property of almost all homo-oligomers characterized to-date. An example of a homo-oligomeric protein is collagen, which is composed of three identical protein chains. Although homo-oligomers often exhibit at least some degree of local asymmetry, this asymmetry generally is limited to small differences in the backbone position, differences in side-chain orientations or limited to a certain part of the protein, while the complex as a whole is still symmetric.

To-date, hetero-oligomeric assemblage of cTRPs have not been generated. The binding to targets, specificity, avidity, thermal stability, solubility, other tertiary structure effects, etc. may be well different from asymmetrical cTRPs and homo-oligomer cTRPs.

Therefore it is an object of the present invention to provide improved cTRPs which are based on assemblage of different oligomers, thereby forming hetero-oligomer cTRPs.

It is another object of the present invention to provide hetero-oligomer cTRPs as vehicles or scaffolds for displaying different therapeutic, prophylactic, and/or diagnostic cargos.

It is another object of the present invention to provide methods of using hetero-oligomer cTRPs for delivery of different cargos in one or more immunization, therapeutics, diagnostics, and/or nanotechnological applications.

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

### SUMMARY OF THE INVENTION

The following embodiments and aspects thereof are described and illustrated in conjunction with compositions and methods which are meant to be exemplary and illustrative, not limiting in scope.

Various embodiments provide asymmetric circular tandem repeat proteins (asymmetric cTRPs), which are assemblage of two or more protomers having amino acid substitutions with cysteine or with non-canonical amino acids at different amino acid residue positions on respective terminal α-helical structural units, thereby facilitating disulfide bonding or click conjugation in between protomers so as to form hetero-oligomer cTRPs with interface bonding/conjugation.

In various embodiments, protomers are provided, which comprise a formula:

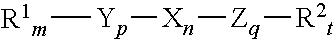

wherein
R¹ is an N-terminal linker or a covalent bond,
R² is a C-terminal linker or a covalent bond,
X is an α-helical peptide repeat unit comprising 30-35 amino acid residues and forming an outer α-helix and an inner α-helix joined by a helix-linker,
Y is a repeat unit having an identical amino acid sequence or at least 90% sequence identity to X except for the substitution of one residue with a cysteine or with a non-canonical amino acid,
Z is a repeat unit having an identical amino acid sequence or at least 90% sequence identity to X except for the substitution of one residue with a cysteine or with a non-canonical amino acid, wherein the cysteine or the non-canonical amino acid in Z is at a different position than in Y, wherein if the substitution in Y is with the cysteine then the substitution in Z is with the cysteine, and wherein if the substitution in Y is with the non-canonical amino acid then the substitution in Z is with the non-canonical amino acid,
m is 0 or 1, wherein if m is 0 then R¹ is absent,
p is 1,
q is 1,
t is 0 or 1, wherein if t is 0 then R² is absent, and
n is a repeat number of the α-helical peptide repeat unit being 4, 1, 2, 6, 10 or 0.

In some embodiments, X in the formula is a polypeptide having an amino acid sequence of any one of SEQ ID NOs: 7 and 54-553. In various aspects, polypeptide X contains an α-helix—turn—α-helix structure, corresponding to a sequence of PEAIKAAAELGKA (SEQ ID NO:15)—GBB type linker—SEEILELLRAAHEL (SEQ ID NO:16), or any of those sequences (first 30 amino acid residues of each) in Table 8. GBB type linker, as a turn between two α-helices, includes but is not limited to GIS; GLD; GKS; GIT; GTT; GYS; GDK; GOE; NDK; GDR; and GDL.

Even when n is 0 (i.e., X is absent), Y and Z are each polypeptides having 30-35 amino acids of an α-helix—turn—α-helix structure, wherein Y and Z are connected by another turn (GBB linker), and Y and Z may otherwise be identical in the α-helix—turn—α-helix region except for substitutions with cysteine or with non-canonical amino acids at different positions in the α-helix—turn—α-helix region.

In some embodiments, Y has an amino acid sequence of SEQ ID NO:6, 9, 11, or 13, or at least 90% sequence identity thereto.

In some embodiments, Z has an amino acid sequence of SEQ ID NO:8, 10, 12, or 14, or at least 90% sequence identity thereto.

In some embodiments, Y and Z of a protomer do not have the substitution with cysteine or with non-canonical amino acid at positions 1 and 3, wherein Y and Z each have a sequence of PEAIKAAAELGKA (SEQ ID NO:15)—GBB type linker—SEEILELLRAAHEL (SEQ ID NO: 16).

In various aspects, a cargo molecule is operably linked to R¹ and/or R². Exemplary cargo molecules include but are not limited to a microbial subunit, a protein, or a small molecule ligand binding domain, such as an ectodomain of a protein. Preferably the protein comprising an immunogen.

In various aspects, non-canonical amino acids, especially those in substituted pairs of repeats in adjacent protomers comprise mutually exclusive orthogonal pair of reactive residues, which are selected from:
one of (S)-2-amino-3-azido-propionic acid or (S)-2-amino-4-azido-butanoic acid and one of (S)-2-aminopent-4-ynoic acid or (S)-2-aminohex-5-ynoic acid;
2-amino-3-mercaptopropanoic acid (L-Cysteine) and one of (S)-2-aminobut-3-enoic acid or (S)-2-aminopro-4-enoic acid;
(S)-2-amino-2-((1R, 8S, 9r)-bicyclo[6.1.0]non-4-yn-9-yl) acetic acid and one of (S)-2-amino-3-azido-propionic acid or (S) 2-amino-4-azido-butanoic acid;
(S)-2-amino-3-(4-(6-methyl-1,2,4,5-tetrazin-3-yl)phenyl) propanoic acid and one of (S)-2-amino-3-cyanopropanoic acid or (S)-2-amino-4-cyanobutanoic acid; and
(S)-2-(N-tert-Butoxycarbonyl)-4-[N-[2-chlorobenzyloxycarbonyl]-N-methoxy]diaminobutanoic acid and (S)-2-amino-3-(3-formylphenyl) propanoic acid.

In various aspects, the number of protomers in forming an cTRP, multiplied by (2+n), equals 24.

In some embodiments, the terminal linkers, as R¹ and/or R², can independently be GGS, GGGS (SEQ ID NO:5), GGSGGGSGGSG (SEQ ID NO:17), GGSGGGSGSG (SEQ ID NO:18), GGSGGGSG (SEQ ID NO:19), GGGGSGGGGS (SEQ ID NO:20), GGGSGGGS (SEQ ID NO: 21), and GGSGGS (SEQ ID NO:22).

Various embodiments provide asymmetrical cTRPs, each of which comprises two or more protomers associated in tandem in a closed circular structure with disulfide bonding and/or click conjugation in between two protomers, preferably in between every two associated protomers.

In some embodiments, an asymmetrical cTRP includes four protomers, each protomer independently comprising a formula of R¹—Y—X₄—Z—R², wherein the cysteine residue or the non-canonical amino acid residue in Z of each protomer forms a covalent bond with the cysteine residue or the non-canonical amino acid residue, respectively, in Y of a next protomer in tandem in the closed circular structure; and wherein each pair of cysteine substitutions in Z of a protomer and Y of a next protomer is different in amino acid residue positions relative to respective protomers, and wherein each pair of non-canonical amino acid residue substitutions in Z of a protomer and Y of a next protomer is a mutually exclusive orthogonal pair of reactive groups for click conjugation.

In some embodiments, an asymmetrical cTRP includes two protomers, each protomer independently comprising a formula of R¹—Y—X₁₀—Z—R², wherein the cysteine or the non-canonical amino acid residue in Z of each protomer forms a covalent bond with the cysteine or the non-canonical amino acid residue, respectively, in Y of a next protomer in tandem in the closed circular structure, and wherein each pair of cysteine substitutions in Z of a protomer and Y of a next protomer is different in amino acid residue positions relative to respective protomers, and wherein each pair of non-canonical amino acid residue substitutions in Z of a protomer and Y of a next protomer is an orthogonal pair of reactive groups for click conjugation.

In some embodiments, an asymmetrical cTRP includes three protomers, each protomer independently comprising a formula of R¹—Y—X₆—Z—R², wherein the cysteine residue or the non-canonical amino acid residue in Z of each protomer forms a covalent bond with the cysteine or the non-canonical amino acid residue, respectively, in Y of a next protomer in tandem in the closed circular structure; and wherein each pair of cysteine substitutions in Z of a protomer and Y of a next protomer is different in amino acid residue positions relative to respective protomers, and wherein each pair of non-canonical amino acid residue substitutions in Z of a protomer and Y of a next protomer is an orthogonal pair of reactive groups for click conjugation.

In some embodiments, an asymmetrical cTRP includes six protomers, each protomer independently comprising a formula of R¹—Y—X₂—Z—R², wherein the cysteine or the non-canonical amino acid residue in Z of each protomer forms a covalent bond with the cysteine or the non-canonical amino acid residue, respectively, in Y of a next protomer in tandem in the closed circular structure; and wherein each pair of cysteine substitutions in Z of a protomer and Y of a next protomer is different in amino acid residue positions relative to respective protomers, and wherein each pair of non-canonical amino acid residue substitutions in Z of a protomer and Y of a next protomer is an orthogonal pair of reactive groups for click conjugation.

In some embodiments, an asymmetrical cTRP includes 12 protomers, each protomer independently comprising a formula of R¹—Y—Z—R², wherein the cysteine or the non-canonical amino acid residue in Z of each protomer forms a covalent bond with the cysteine or the non-canonical amino acid residue, respectively, in Y of a next protomer in tandem in the closed circular structure; and wherein each pair of cysteine substitutions in Z of a protomer and Y of a next protomer is different in amino acid residue positions relative to respective protomers, and wherein each pair of non-canonical amino acid residue substitutions in Z of a protomer and Y of a next protomer is an orthogonal pair of reactive groups for click conjugation.

In various aspects, the substitutions in pairs of Z of a protomer and Y of a next protomer, i.e., interfacing repeats in adjacent protomers, in the closed circular structure are at:
position 1 and position 33,
position 1 and position 2,
position 1 and position 3,
position 1 and position 32,
position 2 and position 33,
position 3 and position 33,
position 3 and position 32,
position 4 and position 3,
position 4 and position 6,
position 12 and position 6,
position 8 and position 9,
position 8 and position 10,
position 8 and position 13,
position 8 and position 6,
position 11 and position 10,
position 11 and position 13,
position 11 and position 15,
position 12 and position 13,
position 17 and position 19,
position 21 and position 23,
position 28 and position 30,
position 4 and position 27,
position 11 and position 15, or
position 4 and position 33,
relative to respective protomers, wherein the amino acid numbering is based on amino acid positions in or aligned with PEAIKAAAELGKA (SEQ ID NO:15)—GBB linker—SEEILELLRAAHEL (SEQ ID NO:16)—GBB linker.

In various embodiments, different cargo molecules are operably linked to the protomers forming an asymmetrical cTRP. In various embodiments, polypeptide sequences of an immunogen of different mammalian species are operably linked to the protomers forming an asymmetrical cTRP. In some embodiments, the cargo molecules are direct genetically ‘fused’ with the protomers, e.g., to be expressed from a same vector. In some embodiments, the cargo molecules are chemically including enzymatically conjugated to the protomers. For example, click chemistry binding members may be modified onto cargo molecules and the protomers, respectively, to facilitate click conjugation between the two. As another example, sortase recognition motifs are fused with cargo molecules and the protomers, respectively, to permit sortase-mediated connection of the two.

Polynucleotides are also provided, which encode one or more protomers disclosed herein.

Vectors are also provided, which include a nucleic acid molecule encoding a protomer disclosed herein.

Genetically engineered cells and/or animals are provided, which has been introduced with one or more vectors each comprising a nucleic acid molecule encoding a protomer disclosed herein.

Various embodiments provide methods of generating monoclonal antibodies cross-protective against a microbe (e.g., influenza), which include:
(a) isolating cells from a sample of blood or other tissue containing cells of the immune system from a subject that has been administered or infected with an asymmetrical circular tandem repeat protein (cTRP) disclosed herein, wherein proteins of two or more strains or subtypes of the microbe are each operably linked to R¹ or R² of the cTRP;
(b) preparing monoclonal antibodies using the cells of (a); and
(c) selecting monoclonal antibodies that cross-react with different strains and subtypes of the microbe.

Additional embodiments provide methods for inducing an immunogenic response to an immunogenic protein from two or more species or to two or more immunogenic proteins, and the methods include administering an asymmetrical circular tandem repeat protein (cTRP) disclosed herein to a subject in need thereof, wherein the immunogenic protein from two or more species or the two or more immunogenic proteins are each operably linked to R¹ or R² of the cTRP.

Some embodiments provide methods of inducing T-cell expansion, which include culturing T-cells in the presence of an asymmetrical circular tandem repeat protein (cTRP) disclosed herein, wherein two or more CD3 engaging domains and one or more co-stimulatory domains are each operably linked to R¹ or R² of the cTRP.

Some embodiments provide methods of inducing engineered T-cell expansion and/or detection, wherein the T-cell is engineered with one or more surface receptors that bind to one or more ligands, and the methods include culturing the engineered T-cell in the presence of an asymmetrical circular tandem repeat protein (cTRP) disclosed herein, wherein the one or more ligands are each operably linked to R¹ or R² of the cTRP.

Some embodiments provide methods of channeling a substrate for reactions mediated by two or more enzymes, comprising contacting the substrate with an asymmetrical circular tandem repeat protein (cTRP) disclosed herein, wherein the two or more enzymes are each operably linked to R¹ or R² of the cTRP.

Some embodiments provide methods of preparing an symmetrical cTRP, which include expressing two or more vectors each comprising a polynucleic aid encoding a different protomer, thereby obtaining two more different protomers; and mixing the two or more different protomers at an equal molar ratio, whereby the two or more different protomers form a closed circular structure, thereby obtaining an asymmetrical cTRP, wherein the two or more different protomers each comprise a polypeptide having the formula of: R¹—Y—Xₙ—Z—R², wherein
R¹ is an N-terminal linker or a covalent bond,
R² is a C-terminal linker or a covalent bond,
X is an α-helical peptide repeat unit comprising 30-35 amino acid residues and forming an outer α-helix and an inner α-helix joined by a helix-linker,
Y is a repeat unit having an identical amino acid sequence as X except for substitution of one residue with a cysteine or with a non-canonical amino acid and optionally further except for deletion of an amino acid residue at the terminus outside of the outer and inner α-helices,
Z is a repeat unit having an identical amino acid sequence as X except for substitution of one residue with a cysteine or with a non-canonical amino acid and optionally further except for deletion of an amino acid residue at the terminus outside of the outer and inner α-helices, wherein the cysteine or the non-canonical amino acid in Z is at a different position than in Y, wherein if the substitution in Y is with the cysteine then the substitution in Z is with the cysteine, and wherein if the substitution in Y is with the non-canonical amino acid then the substitution in Z is with the non-canonical amino acid, and
n is a repeat number of the α-helical peptide repeat unit being 0, 1, 2, 4, 6 or 10; and
wherein the substitutions with cysteines are at different amino acid positions in each Y and Z of the two or more different protomers, or wherein the substitutions with the non-canonical amino acids in each pair of Z of a protomer and Y of a next protomer are with mutually exclusive orthogonal pair of reactive residues for click conjugation.

Some embodiments provide methods of preparing a multivalent vaccine, which include: preparing an asymmetrical circular tandem repeat protein (cTRP), wherein proteins of two or more different strains or subtypes of a virus or proteins of two or more different viruses are each operably linked to R¹ or R² of a protomer. In some embodiments where the virus comprises an influenza, each protein of the influenza is fused to a trimerization domain, and the method further comprises inducing trimerization of each protein of the influenza, followed by conjugating the trimerized proteins of the influenza to R¹ or R² of a different protomer, before mixing the two or more different protomers to form the asymmetrical cTRP. In further embodiments, each protein of the influenza is further fused with a sortase recognition motif LPXTG (SEQ ID NO:44) at the C-terminus, and R¹ comprises a sortase recognition motif GGG or pentaglycine or diglycine. In other embodiments, each protein of the influenza is further fused with a sortase recognition motif GGG or pentaglycine or diglycine at the N-terminus, and R² comprises a sortase recognition motif LPXTG (SEQ ID NO:44). In some instances, the methods further include adding sortase to a mixture containing the sortase recognition motif-fused influenza and the sortase recognition motif-fused protomer/toroid.

Other features and advantages of the invention will become apparent from the following detailed description, taken in conjunction with the accompanying drawings, which illustrate, by way of example, various features of embodiments of the invention.

### BRIEF DESCRIPTION OF DRAWINGS

Exemplary embodiments are illustrated in referenced figures. It is intended that the embodiments and figures disclosed herein are to be considered illustrative rather than restrictive.

FIGS. 1A-1D. Asymmetric (aSym) cTRP assembly. FIG. 1A Cartoon depiction of aSym_cTRP design. FIG. 1B Crude Ni-purified aSym_cTRP in SDS PAGE under non-reducing (NR) and reducing (R) conditions. aSym_cTRP runs at expected molecular weight (mw) under non-reducing condition and reduces into individual protomers under reducing condition. FIG. 1C Densitometry of reduced SDS PAGE lane in (FIG. 1B) supports 1:1:1:1 stoichiometry of the protomers. FIG. 1D Native size exclusion chromatography (SEC) of crude Ni-purified aSym_cTRP runs at the predicted mw of a 1:1:1:1 tetrameric assembly.

FIGS. 2A-2C. CD3 Immunogens. FIG. 2A. 3D model of CD3 δε heterodimer in complex with TCR and additional CD3 γ/ε and ζ subunits. FIG. 2B. 3D model of Llama Fc scaffolded soluble CD3 δε design. FIG. 2C. 3D model of toroid tetramer design presenting human/cyno/canine/murine CD3 heterodimers.

FIG. 3. B cell receptor clustering by aSym_cTRP. From left to right: 1. cartoon of an unbound B cell receptor. 2. B cell receptor interacting with a monomeric antigen, transducing a weak signal. 3. The aSym_cTRP displaying four distinct antigens bound to a single B cell receptor monomerically, transducing a weak signal. 4. The aSym_cTRP interacting with a B cell receptor capable of recognizing a conserved epitope shared by all antigens driving B cell receptor clustering and strong expansion and class switching signals.

FIG. 4. The sequences of cTRP24(6)SS protomers (denoted with a numerical appendix to “aSym”, e.g., “aSym1” or “aSym_1”) are provided: cTRP24(6)SS_aSym1 (SEQ ID NO:1), cTRP24(6)SS_aSym2 (SEQ ID NO: 2), cTRP24(6)SS_aSym3 (SEQ ID NO: 3), and cTRP24(6)SS_aSym4 (SEQ ID NO: 4). In the sequences, the position of cysteine mutations facilitating defined 1:1:1:1 hetero-complexes are indicated with bolded/underlined letters “C.” Amino acids are color-coded in each sequence to denote the unique interfaces of each protomer, with like-colored regions pairing with each other.

FIG. 5. Quad-CAR T Cell Expansion and Screening. Left to right: a series of four cTRP tetramers each presenting a single ectodomain for a given CAR on a Quad-CAR T cell. The aSym_cTRP displaying a single copy of each distinct ectodomain recognized by a Quad-CAR T cell.

FIG. 6. Superose 6 size exclusion chromatography and SDS PAGE of purified thymic stromal lymphopoietin receptor (TSLPR) aSym cTRPs assembled from protomers shown in Table 1. The schematic on top right shows a predicted structure of tetrameric aSym cTRP where each subunit is fused with TSLPR from one species (either human, cynomolgus monkey, mouse, or canine) to produce a multi-species TSLPR immunogen. The left panel shows the SEC-HPLC analysis of a fully assembled multi-species TSLPR immunogen after purification by Ni-affinity and size-exclusion techniques. The right panel shows SDS-PAGE analysis of fully assembled multi-species TSLPR immunogen under native and reducing conditions. “asym_cTRP tetramer” refers to the band corresponding to the fully assembled multi-species TSLPR immunogen and “monomer” refers to the band corresponding to the individual subunits of the multi-species TSLPR immunogen. The purified protein under non-reducing SDS PAGE runs at the correct size for the tetrameric complex and indicates proper disulfide connectivity. Upon reduction the complex breaks down into the correct size predicted for the monomeric protomers. Multi-species immunogen with mouse TSLPR on subunit no. 2 (left, upper panel) multi-species immunogen with rat TSLPR on subunit no. 2 (left, lower panel). SDS PAGE of multi-species immunogen with mouse TSLPR on subunit no. 2 (left two lanes next to ladder lane) multi-species immunogen with rat TSLPR on subunit no. 2 (right two lanes).

FIG. 7A. Illustration of 1:1:1:1 stoichiometry and validation of TSLPR multi-species immunogen. Purified Canine/Mouse/Cyno/Human aSym cTRP formed a complex by super-shift assay with each of anti-His, anti-murine TSLPR and anti-StrepII Abs. 2× molar excess of antibody was incubated with 1× aSym cTRP and the percent antibody shifted (approx. 50%) indicates the complex is the expected 1:1 stoichiometry. (Cartoon on left) Top to bottom are individual assay conditions. Asymmetric cTRP only, Asymmetric cTRP+antibody against tag on subunit no. 1, Asymmetric cTRP+antibody against mouse TSLPR on subunit no. 2, Asymmetric cTRP+antibody against tag on subunit no. 4. (Graphs on right) SEC-HPLC analyses of fully assembled multi-species TSLPR immunogen (human, cyno, murine, canine) and three SEC-HPLC analyses of fully assembled multi-species TSLPR immunogen after incubation with a commercial antibody that recognizes a unique subunit in the complex (bottom three). Zoomed inset shows area under the curve calculation method for percent antibody bound by asymmetric cTRP and corresponding results are annotated. Multi-species TSLPR immunogen cartoon (top left) illustrates the tags fused to c-terminal end of a subunits 2, 3 and 4 respectively. “aSym_cTRP only” refers to SEC-HPLC trace of multi-species TSLPR immunogen alone. “anti-His Ab” and “anti-His Ab+aSym_cTRP” refers to SEC-HPLC of two-fold molar excess of an anti-Histidine commercial antibody relative to multi-species TSLPR immunogen, alone or in combination with multi-species TSLPR immunogen respectively. “anti-Mouse Ab” and “anti-Mouse Ab+aSym_cTRP” refers to SEC-HPLC of two-fold molar excess of an anti-murine TSLPR commercial antibody relative to multi-species TSLPR immunogen, alone or in combination with multi-species TSLPR immunogen respectively. “anti-Strep Ab” and “anti-Strep Ab+aSym_cTRP” refers to SEC-HPLC of two-fold molar excess of an anti-Streptavidin commercial antibody relative to multi-species TSLPR immunogen, alone or in combination with multi-species TSLPR immunogen respectively. The shift to the left of aSym_cTRP in the antibody+aSym_cTRP condition relative to aSym_cTRP only condition indicate an increase in molecular weight of aSym_cTRP as a result of binding to the subunit-specific antibody. A 50% reduction in the quantity of antibody in the antibody+aSym_cTRP condition relative to the antibody only condition indicates binding of exactly one aSym_cTRP subunit to the subunit specific antibody.

FIG. 7B. Sequence homology of the full TSLPR domains across four preclinically relevant species, with the highlighted regions show the fully conserved sequence between the four species; bottom is calculated TSLPR sequence similarity between two species.

FIG. 7C. Anti-TSLPR campaign by immunizing OMNIRAT® and analysis of total serum titers and cross-reactive serum titers. Middle panel shows the presence of target specific antibodies (“antibody titer”) in the serum of the immunized animals as detected by an enzyme-linked immunosorbent assay (ELISA). Each bar corresponds to independent ELISA tests conducted by coating plates with the ecto-domains of either human TSLPR antigen (“Human”) or cyno TSLPR (“Cyno”) antigen or murine TSLPR antigen (“Murine) or canine TSLPR antigen (“Canine”). In addition, ELISA tests were conducted by coating plates with both cyno and human TSLPR antigens (“Human+Cyno”) or murine and human TSLPR antigens (“Human+Murine”) or canine and human TSLPR antigens (“Human+Canine”) to estimate species cross-reactive titer. ELISA against “BSA” was performed to show non-specific antibody titer. Bottom panel shows the relative binding of a commercial human TSLPR antibody (Clone 1B4 from Biolegend) to each target antigen or combination thereof mentioned above, performed as a positive and negative control to the rat antibody titer ELISA assay.

FIG. 7D. (top) shows the antibody titer that is specific to human and one other species (“cross-reactive titer”) as a percentage of the human-specific antibody titer, calculated as described in the example; and (bottom) shows the corresponding calculation for commercial human TSLPR antibody control.

Cross-reactive titer can be calculated as follows, using the calculation of percentage of human titer that is also cross-reactive with cyno as an example:

$${Titer}_{human - \& cyno - specific cross - reactive} = [ \text{} {Titer}_{ecto \_ human - TSLPR} + {Titer}_{ecto \_ cyno - TSLPR} ] - {Titer}_{ecto \_ human - TSLPR \& ecto \_ cyno - TSLPR}$$

$$And$$

$$Human Titer ( \% cross - reactive , to cyno ) = \frac{{Titer}_{human - \& cyno - specific cross - reactive}}{{Titer}_{ecto \_ human - TSLPR}} \times 100$$

FIG. 7E. (Left) shows the number of human TSLPR only reactive versus human and murine TSLPR cross-reactive B cells per 10,000 B cells isolated from the immunized rats and screened for reactivity with a BEACON® instrument. (Right) Species reactivity for isolated and recombinantly produced antibodies identified from the rat immunization campaign as determined by FACS and ELISA assay.

FIG. 8A-8V. Exemplary repeat (α-helical structure unit) sequences.

FIG. 9. Exemplary Cysteine mutation pairs.

FIGS. 10A-10B. Asymmetrical cTrp linking and cargo decoration. FIG. 10A. One-pot tetramer linkage. FIG. 10B. Site-specific cargo binding.

FIG. 11. Protomer sequences that may be assembled at 1:1:1:1 into asymmetric, disulfide bonded cTRPs that display PD-L1 ectodomains from four different species on each asymmetric cTRP, hence four immunogens presented by each cTRP. The PD-L1 ectodomain is indicated in italics and underlined. A 18-amino acid signal peptide is indicated on the N-terminus of respective PD-L1 ectodomain. The positions of cysteine mutations at terminal repeats of α-helical structure unit are indicated with bolded/underlined letters “C.” Each cTRP may be formed from the assemblage of:
Toroid_cfPDL1-asym1_cTRP24(6)SS_Tetramer-His ((SEQ ID NO:28) a canine PD-L1 immunogen with a His tag),
Toroid_mmPDL1-aSym2_cTRP24(6)SS_Tetramer ((SEQ ID NO:29) a mouse PD-L1 immunogen),
Toroid_mfPDL1-aSym3_cTRP24(6)SS_Tetramer-sFLAG ((SEQ ID NO:31) a cynomolgus PD-L1 immunogen with a sFLAG tag), and
Toroid_hsCRLF2-aSym4_cTRP24(6)SS_Tetramer-StrepII ((SEQ ID NO:32) a human PD-L1 immunogen with a StrepII tag);
   or from the assemblage of:
Toroid_cfPDL1-asym1_cTRP24(6)SS_Tetramer-His ((SEQ ID NO:28) a canine PD-L1 immunogen with a His tag),
Toroid_rnPDL1-aSym2_cTRP24(6)SS_Tetramer ((SEQ ID NO:30) a rat PD-L1 immunogen),
Toroid_mfPDL1-aSym3_cTRP24(6)SS_Tetramer-sFLAG ((SEQ ID NO:31) a cynomolgus PD-L1 immunogen with a sFLAG tag), and
Toroid_hsCRLF2-aSym4_cTRP24(6)SS_Tetramer-StrepII ((SEQ ID NO:32) a human PD-L1 immunogen with a StrepII tag).

FIGS. 12A-12B. SEC analysis (12A) and SDS PAGE analysis (12B) of multi-species PD-L1-displaying aSym cTRP formed based on protomer sequences shown in FIG. 11.

FIG. 13. Amino acid sequences encoded by vectors that encode for four protomers with a signal sequence (e.g., Ig kappa leader sequence of SEQ ID NO:37), a furin cleavage site (e.g., SEQ ID NO:39), a sortase recognition N-terminal motif of tri-glycine, and one or more affinity tags, wherein the four protomers have different residue positions of cysteine substitutions at terminal repeats, and the four protomers can assemble in a cTRP at a molar ratio of approximately 1:1:1:1.

FIG. 14. Amino acid sequences encoded by vectors that each encode for a different exemplary influenza HA sequence with a signal sequence (e.g., Ig kappa leader sequence of SEQ ID NO: 37), a fibritin foldon trimerization domain (SEQ ID NO:42), and a sortase recognition C-terminal motif (e.g., a sequence of SEQ ID NO:43), and one or more affinity tags.

FIGS. 15A-15C. SEC analysis (15A) and SDS-PAGE analysis (15B) of mammalian cell expressed sortase; and SDS-PAGE analysis (15C) demonstrating sortase conjugation of Nb14-LPETG (SEQ ID NO:43) and GGG-eGFP or GGG-mScarlet.

### DETAILED DESCRIPTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, NY 2006); March, Advanced Organic Chemistry Reactions, Mechanisms and Structure 7^{th} ed., J. Wiley & Sons (New York, NY 2013); and Sambrook and Russel, Molecular Cloning: A Laboratory Manual 4^{th} ed., Cold Spring Harbor Laboratory Press (Cold Spring Harbor, NY 2012), provide one skilled in the art with a general guide to many of the terms used in the present application. For references on how to prepare antibodies, see D. Lane, Antibodies: A Laboratory Manual 2^{nd} ed. (Cold Spring Harbor Press, Cold Spring Harbor NY, 2013); Kohler and Milstein, (1976) Eur. J. Immunol. 6:511; Queen et al. U.S. Pat. No. 5,585,089; and Riechmann et al., Nature 332:323 (1988); U.S. Pat. No. 4,946,778; Bird, Science 242:423-42 (1988); Huston et al., Proc. Natl. Acad. Sci. USA 85:5879-5883 (1988); Ward et al., Nature 334:544-54 (1989); Tomlinson I. and Holliger P. (2000) Methods Enzymol, 326, 461-479; Holliger P. (2005) Nat. Biotechnol. September; 23(9):1126-36).

One skilled in the art will recognize many methods and materials similar or equivalent to those described herein, which could be used in the practice of the present invention. Indeed, the present invention is in no way limited to the methods and materials described. For purposes of the present invention, the following terms are defined below.

As used herein, the term “protomer” refers to the structural unit of an oligomeric protein (Chetverin, A. B. (1986) FEBS Lett. 196(1): 121-125). A protomer is a protein unit, wherein two or more copies of identical protein units form a homo-oligomer, or different protein units form a larger hetero-oligomer. Protomers usually arrange in cyclic symmetry. Relative to the formed, larger oligomer, a protomer may also be understood as a protein subunit.

The term “non-canonical amino acid” (ncaa) refers to amino acids that are not among the twenty canonical amino acids nor their 19 D-stereoisomeric counterparts. Many ncaa are found in proteins with moderate frequency and they can be very effective at improving folding and binding properties of proteins and peptidomimetics.

The term “operably linked” refers to a linkage of two elements in a functional relationship. For example, “operably linked” may refer to a linkage of polypeptide elements in a functional relationship. “Operably-linked” also refers to the association of two chemical moieties so that the function of one is affected by the other, e.g., an arrangement of elements wherein the components so described are configured so as to perform their usual function.

“Variants” include protein sequences having one or more amino acid additions, deletions, stop positions, or substitutions, as compared to a protein sequence disclosed elsewhere herein.

An amino acid substitution can be a conservative or a non-conservative substitution. Variants of protein sequence disclosed herein can include those having one or more conservative amino acid substitutions. A “conservative substitution” or “conservative amino acid substitution” involves a substitution found in one of the following conservative substitutions groups: Group 1: Alanine (Ala; A), Glycine (Gly; G), Serine (Ser; S), Threonine (Thr; T); Group 2: Aspartic acid (Asp; D), Glutamic acid (Glu; E); Group 3: Asparagine (Asn; N), Glutamine (Gln; Q); Group 4: Arginine (Arg; R), Lysine (Lys; K), Histidine (His; H); Group 5: Isoleucine (Ile; I), Leucine (Leu; L), Methionine (Met; M), Valine (Val; V); and Group 6: Phenylalanine (Phe; F), Tyrosine (Tyr; Y), Tryptophan (Trp; W).

Additionally, amino acids can be grouped into conservative substitution groups by similar function, chemical structure, or composition (e.g., acidic, basic, aliphatic, aromatic, or sulfur-containing). For example, an aliphatic grouping may include, for purposes of substitution, G, A, V, L, and I. Other groups including amino acids that are considered conservative substitutions for one another include: sulfur-containing: M and C; acidic: D, E, N, and Q; small aliphatic, nonpolar or slightly polar residues: A, S, T, P, and G; polar, negatively charged residues and their amides: D, N, E, and Q; polar, positively charged residues: H, R, and K; large aliphatic, nonpolar residues: M, L, I, V, and C; and large aromatic residues: F, Y, and W.

Non-conservative substitutions include those that significantly affect: the structure of the peptide backbone in the area of the alteration (e.g., the α-helical or beta-sheet structure); the charge or hydrophobicity of the molecule at the target site; or the bulk of the side chain. Non-conservative substitutions which in general are expected to produce the greatest changes in the protein's properties are those in which (i) a hydrophilic residue (e.g. S or T) can be substituted for (or by) a hydrophobic residue (e.g. L, I, F, V, or A); (ii) a C or P can be substituted for (or by) any other residue; (iii) a residue having an electropositive side chain (e.g. K, R, or H) can be substituted for (or by) an electronegative residue (e.g. Q or D); or (iv) a residue having a bulky side chain (e.g. F), can be substituted for (or by) one not having a bulky side chain, (e.g. G). Additional information is found in Creighton (1984) Proteins, W.H. Freeman and Company.

Variants of protein sequences disclosed herein also include proteins with at least 70% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to a protein sequence disclosed herein.

“Percent (%) amino acid sequence identity” with respect to the sequences identified herein is defined as the percentage of amino acid residues in a candidate sequence that are identical with the amino acid residues in the reference sequence after aligning the sequences and introducing gaps, if necessary, to achieve the maximum percent sequence identity. Alignment for purposes of determining percent amino acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared. For example, % amino acid sequence identity values generated using the WU-BLAST-2 computer program (Altschul et al., Methods in Enzymology, 266:460-480 (1996)) uses several search parameters, most of which are set to the default values. Those that are not set to default values (i.e., the adjustable parameters) are set with the following values: overlap span=1, overlap fraction=0.125, word threshold (T)=11 and scoring matrix BLOSUM62.

Repeat proteins are formed by repetition of modular units of protein sub-structures. The overall structural architecture of repeat proteins is dictated by the internal geometry of the protein and the local packing of the repeat building blocks. These features are generated by underlying patterns of amino acid sequences, that themselves, are repetitive in nature.

Naturally existing repeat proteins play important biological roles as macromolecular binding and scaffolding domains, enzymes, and building blocks for the assembly of fibrous materials. The structure and identity of these repeat proteins are highly diverse, ranging from extended, super-helical folds that bind peptide, DNA, and RNA partners, to closed and compact conformations with internal cavities suitable for small molecule binding and catalysis.

The current disclosure provides circular tandem repeat proteins (cTRPs) designed purely by geometric criteria defining the inter-repeat geometry, without reference to the sequences or the structures of naturally existing repeat protein families. Because the design methodology did not rely on template sequence or structural information taken from natural repeat proteins, the resulting cTRPs are unlike those seen in nature. More particularly, the designed cTRPs have highly repetitive α-helical structures that are weakly interacting and have covalent bonds to help further stabilize the structure. The inter-repeat packing geometry is constrained so as to juxtapose the N- and C-termini creating circular architectures. Further, the repeated domains including the circular α-helical repeat proteins are uniformly handed. These proteins are capable of independently self-folding and have high thermostability and solubility.

The disclosed cTRPs can have numerous uses as new biomaterials. For example, the cTRPs have a number of uses as scaffolds for geometrically precise, arrayed presentation of cell-signaling and/or immune-related protein and peptide epitopes, as well as numerous other therapeutic, diagnostic, and nanotechnological uses. In particular embodiments, functional domains can be inserted between or within α-helical sequences and linker sequences without significantly altering the engineered parameters (e.g., circular, handed, α-helical repeats) of the cTRPs. The cTRPs can also be used as nano-filters. The cTRPs, methods of generation, characteristics, and uses are described in detail below.

cTRPs disclosed herein has a circular tandem repeat architecture, assembled from two or more protomers, each containing repetitive α-helical structures joined by loops. In various embodiments, the two or more protomers are different from each other in at least the N-terminal repeats and the C-terminal repeats. In various embodiments, the differences are differences in the N-terminal repeats across two or more protomers, and differences in the C-terminal repeats across two or more protomers, as well as the difference between the N-terminal repeat and the C-terminal repeat of a same protomer. Hence, the differences permit specific associations at interfaces between specific protomers, so as to arrange the two or more different protomers into a circular structure with a specific sequence. Particularly, the association between two protomers is preferably based on disulfide bonding, or may be based on click chemistry bonding.

Therefore, in various embodiments, the N-terminal repeat and the C-terminal repeat of a protomer each has a cysteine substitution, i.e., a non-cysteine amino acid is mutated into a cysteine, preferably at a different residue/location in respective α-helical repeat and the mutated residue being one on surface of respective α-helical repeat to interface with another protomer. In preferable embodiments, a cysteine substitution at the C-terminal repeat of a first protomer is capable of forming a disulfide bonding with a cysteine substitution at the N-terminal repeat of a second protomer, when the distance between these two substitution residues upon assembly of the first and the second (or more) protomers into a cTRP are within a certain threshold (e.g., 7.0 Å or less, 6.5 Å or less, 6.0 Å or less, 5.6 Å or less, 5.0 Å or less, 4.5 Å or less, 4.0 Å or less, 3.5 Å or less, or between 3.0 and 7.0 Å). Likewise, a cysteine substitution at the C-terminal repeat of the second protomer is capable of forming a disulfide bonding with a cysteine substitution at the N-terminal repeat of a third protomer, and so on. In some embodiments, when the first and the second protomer each contains 12 repeats and together form a cTRP of 24 repeats, the substitution at the C-terminal repeat of the second protomer is capable of forming a disulfide bonding by pairing with the cysteine substitution at the N-terminal repeat of the first protomer.

Preferably besides the difference in cysteine substitution positions in respective α-helix in the terminal repeats of a same protomer, two or more protomers will each have a different combination of substitution positions in the terminal repeats. For example, protomer A has a substitution at a residue position a₁ in the α-helix of the N-terminal repeat and a substitution at residue position a₂ in the α-helix of the C-terminal repeat, a₁ and a₂ being different positions relative to respective α-helix (i.e., a₁≠a₂); protomer B has a substitution at residue position b₁ in the α-helix of the N-terminal repeat and a substitution at residue position b₂ in the α-helix of the C-terminal repeat, b₁ and b₂ being different positions relative to respective α-helix (i.e., b₁≠b₂); protomer C has a substitution at residue position c₁ in the α-helix of the N-terminal repeat and a substitution at residue position c₂ in the α-helix of the C-terminal repeat, c₁ and c₂ being different positions relative to respective α-helix (i.e., c₁≠c₂); and protomer D has a substitution at residue position d₁ in the α-helix of the N-terminal repeat and a substitution at residue position d₂ in the α-helix of the C-terminal repeat, d₁ and d₂ being different positions relative to respective α-helix (i.e., d₁≠d₂); and so on; and an asymmetric cTRP (also referred to as hetero-oligomer cTRP) is formed from a circular assemblage of the protomers as: A⋅⋅B⋅⋅C⋅⋅D⋅ . . . ⋅A, specifically a₁-a₂⋅⋅b₁-b₂⋅⋅c₁-c₂⋅⋅d₁-d₂⋅⋅a₁-a₂, wherein “⋅⋅” represents disulfide bonding (based on cysteine substitutions) or click chemistry bonding (based on non-canonical amino acid substitutions, e.g., wherein the non-canonical amino acid is modified with a member of a click chemistry bonding pair) between terminal repeats of adjacent protomers, hence “substitution-facilitated bonding”, and “-” represents peptidyl bonds (e.g., a₁-a₂ is one polypeptide), and wherein for each substitution-facilitated bonding pair (residues connected by “ . . . ”), their residue positions are: a₂≠b₁, b₂≠c₁, c₂·d₁, d₂≠ . . . ≠a₁. In some embodiments, both of a₁ and a₂ are different from b₁ and b₂, and so on; that is, a₁≠a₂≠b₁≠b₂≠c₁≠c₂≠d₁≠d₂, and so on.

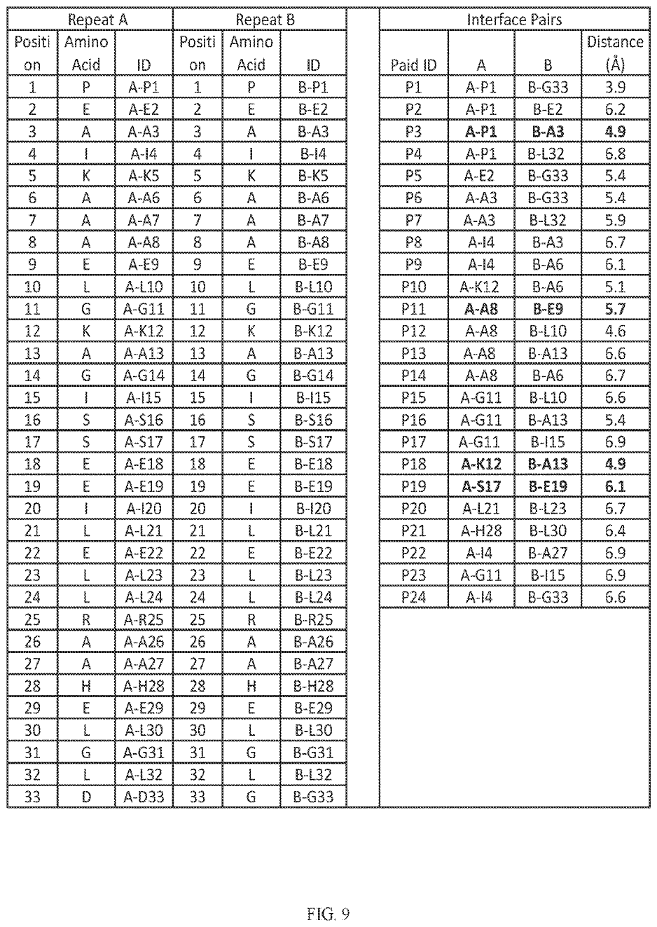

In various embodiments, residue positions of substitution-facilitated bonding pair, i.e., a₂ and b₁, b₂ and c₁, c₂ and d₁, and d₂ . . . a₁ (i.e., a fourth pair, d₂ and a₁, for a hetero-tetramer cTRP), are selected from those with a measured distance smaller than 7.0 Å in FIG. 9.

In some embodiments, for assemblage of a hetero-tetramer cTRP (i.e., depicted by a₁-a₂⋅⋅b₁-b₂⋅⋅c₁-c₂⋅⋅d₁-d₂⋅⋅a₁-a₂), four protomers contain amino acid substitutions (e.g., with cysteine, or with a non-canonical amino acid) at terminal repeats at following positions:
a₂ is for the Alanine at residue position 13 (A13) of the C-terminal α-helix repeat having an amino acid sequence of PEAIKAAAELGKA (SEQ ID NO:15)—GBB linker—SEEILELLRAAHEL (SEQ ID NO:16) of a first protomer, and b₁ is for the Lysine at residue position 12 (K12) of the N-terminal α-helix repeat having an amino acid sequence of PEAIKAAAELGKA (SEQ ID NO: 15)—GBB linker—SEEILELLRAAHEL (SEQ ID NO: 16) of a second protomer;
b₂ is for the glutamic acid at residue position 9 of the C-terminal α-helix repeat having an amino acid sequence of PEAIKAAAELGKA (SEQ ID NO:15)—GBB linker—SEEILELLRAAHEL (SEQ ID NO:16) of the second protomer, and c₁ is for the Alanine at residue position 8 (A8) of the N-terminal α-helix repeat having an amino acid sequence of PEAIKAAAELGKA (SEQ ID NO:15)—GBB linker—SEEILELLRAAHEL (SEQ ID NO: 16) of a third protomer;
c₂ is for the Glutamic acid at residue position 19 (E19) of the C-terminal α-helix repeat having an amino acid sequence of PEAIKAAAELGKA (SEQ ID NO:15)—GBB linker—SEEILELLRAAHEL (SEQ ID NO: 16) of the third protomer, and d₁ is for the Serine at residue position 17 (S17) of the N-terminal α-helix repeat having an amino acid sequence of PEAIKAAAELGKA (SEQ ID NO:15)—GBB linker—SEEILELLRAAHEL (SEQ ID NO:16) of a fourth protomer; and
d₂ is for the Alanine at residue position 3 (A3) of the C-terminal α-helix repeat having an amino acid sequence of PEAIKAAAELGKA (SEQ ID NO:15)—GBB linker—SEEILELLRAAHEL (SEQ ID NO:16) of the fourth protomer, and a₁ is for the Proline at position 1 (P1) of the N-terminal α-helix repeat having an amino acid sequence of PEAIKAAAELGKA (SEQ ID NO: 15)—GBB linker—SEEILELLRAAHEL (SEQ ID NO: 16) of the first protomer.

In various instances, if a distance between two residues, one on each terminal α-helix repeat of adjacent protomers in a cTRP, is within a threshold effective for inducing disulfide bonding or click chemistry bonding, the two residues can be selected for the cysteine substitution or for the non-canonical amino acid substitution. Preferably, the two residues (one on each α-helix terminal repeat of adjacent protomers) are at different positions relative to respective α-helix terminal repeat. In some embodiments, the two residues (one on each α-helix terminal repeat of adjacent protomers) are at positions off by 1 residue in respective α-helices. In some embodiments, the two residues (one on each α-helix terminal repeat of adjacent protomers) are at positions off by 2 residues in respective α-helices. In some embodiments, the two residues (one on each α-helix terminal repeat of adjacent protomers) are at positions off by 3 residues in respective α-helices. In some embodiments, the two residues (one on each α-helix terminal repeat of adjacent protomers) are at positions off by 4 residues in respective α-helices. In some embodiments, the two residues (one on each α-helix terminal repeat of adjacent protomers) are at positions off by 5 residues in respective α-helices. In some embodiments, the two residues (one on each α-helix terminal repeat of adjacent protomers) are at positions off by 6 residues in respective α-helices. In some embodiments, the two residues are at the far-most positions within the distance threshold on respective α-helices.

Each repetitive α-helical structure includes an outer α-helix and an inner α-helix, connected by a loop (i.e., a turn conformation) between the outer α-helix and the inner α-helix. In particular embodiments, an α-helical structure is repetitive (e.g., structurally repetitive) when following (i) stacking with an adjacent α-helical structure; and (ii) comparison using root-mean-square-deviation (RMSD), the distance between corresponding atoms of the stacked outer α-helices and the stacked inner α-helices is within 2 angstrom (Å); within 1.5 Å; within 1 Å; within 0.5 Å; within 0.4 Å; or within 0.2 Å.

Typically, each repetitive α-helical structure is based on a polypeptide sequence folded into a bundle containing two antiparallel α-helices. The two segments forming the antiparallel helices within each repetitive structure can each be independently about 10-17 or about 12-15 or about 13-14 residues. Thus, in particular embodiments, an asymmetric/hetero-oligomeric cTRP disclosed herein corresponds to a circular tandem repeat architecture containing 24 α-helical structural units in 2-12 segments, folded into a ring with outer α-helices and inner α-helices, wherein each segment contains an equal number of identical (or repetitive) α-helical structure units, and the α-helical structure units are different for each segment at least in the terminal α-helical structure units, and the total number of α-helical structure units in the 2-12 segments are 24. In some embodiments, a cTRP disclosed herein contains 24 α-helical structural units in 2 segments of 12 α-helical structural units. In some embodiments, a cTRP disclosed herein contains 24 α-helical structural units in 3 segments of 8 α-helical structural units. In some embodiments, a cTRP disclosed herein contains 24 α-helical structural units in 4 segments of 6 α-helical structural units. In some embodiments, a cTRP disclosed herein contains 24 α-helical structural units in 6 segments of 4 α-helical structural units. In some embodiments, a cTRP disclosed herein contains 24 α-helical structural units in 8 segments of 3 α-helical structural units. In some embodiments, a cTRP disclosed herein contains 24 α-helical structural units in 12 segments of 2 α-helical structural units.

Disclosed engineered cTRPs that contain a circular architecture represent a target for rational, geometry-guided design of structural repeats for several reasons. Closure results from simple constraints on the inter-repeat geometry: if the transformation between successive repeats is considered as being composed of a rotation (curvature) about an axis together with a translation (rise) parallel to that axis, then the rise must equal zero and the curvature multiplied by the number of repeats must equal 360°. Circular structures are stabilized by interactions between the first and last repeats, obviating the need for capping repeats to maintain solubility and making them more tolerant than open repeat architectures to imperfections in the designed geometry. Thus, in particular embodiments, a circular protein (e.g., a protein having a circular architecture) is one wherein the N-terminal and C-terminal ends of the protein are naturally found within 10 Å following expression and folding. In particular embodiments, a circular protein is one wherein the N-terminal and C-terminal ends of the protein are naturally found within 10 Å; within 9 Å; within 8 Å; within 7 Å; within 6 Å; within 5 Å; within 4 Å; within 3 Å; within 2 Å; within 1 Å; or within 0.5 Å; following expression and folding. Naturally found means that the cTRP is self-folding. In particular embodiments, a circular protein is one designated as such by the teachings of Kajava, A. V. Tandem repeats in proteins: from sequence to structure. J. Struct. Biol. 179, 279-288, doi: 10.1016/j.jsb.2011.08.009 (2012).

cTRPs offer the advantages of rotational symmetry (for example, in generating higher-order assemblies) with the added control provided by a covalent linkage between repetitive α-helical structures (also called “repeats” or “repeat units”).

In certain embodiments, linkers between the two antiparallel α-helices within an α-helical structure can utilize a GBB format, also call GBB linker or GBB type linker. In particular embodiments, the G residue is glycine. In particular embodiments, the G residue is not isoleucine or valine. In particular embodiments, the B residues are selected from serine, threonine, asparagine, or glutamine. Examples of GBB linkers include GLD, GLN, GLS, GLT, GID, GIN, GIT, GIS, GVD, GVN, GVT, GVS, GTD, GTN, GTT, GTS, GKS, GYS, and GNS. As will be understood by one of ordinary skill in the art, particular residues that fall within a G or B classification can depend on the particular protein at issue. Therefore, while representative (and common) selection options within these groups are provided, such examples are not exclusive to use of other potential residues. Without being bound by theory, and in particular embodiments, GBB linkers are used because they facilitate formation of left-handed proteins. It is important to note that, due to the circular architecture of the disclosed repeat proteins, the repeat proteins can “begin” or “end” with any residue at the N- or C-terminus.

The present disclosure relates to hetero-oligomeric cTRPs. The present invention provides unique cTRP disulfide pairings that facilitate defined hetero-oligomer toroid formation. In particular, mutations that facilitate specific cTRP repeat pairing have been engineered that allow for the generation of defined asymmetric toroid structures. These cTRPs with defined asymmetry can be used in immunogen design for presentation of multi-species antigens that facilitate discovery of cross-reactive binders; multivalent vaccines that improve broadly neutralizing antibody responses; generation of T cell expansion reagents that target multiple co-stimulatory targets; and/or natural production creation through multi-enzyme complexes that improve catalytic efficiency of the engineered biosynthetic pathway.

The invention includes a new composition of matter that improves the utility of existing cTRP technology. The conceived disulfide mutations allow for the defined paring of cTRP protomers. This allows for the controlled generation of cTRP hetero-oligomers with defined 1:1, 1:1:1, 1:1:1:1, 1:1:1:1:1:1, 1:1:1:1:1:1:1:1, or 1:1:1:1:1:1:1:1:1:1:1:1 stoichiometries. Exemplary interface pairs are provided in FIG. 9 (right panel).

### Protomers

In certain embodiments, the asymmetric cTRPs contain protomers of any repeat length that is evenly divisible into 24 and sums to 24, i.e., have a total of 24 α-helical structure units, where the cTRPs are made up of the following combinations of protomers:
24 different protomers, each being a different sequence folded into an α-helical structure unit;
12 pairs of protomers, each being a different sequence folded into two α-helical structure units in tandem,
8 triplets of protomers, each being a different sequence folded into three α-helical structure units in tandem,
6 quadruplets of protomers, each being a different sequence folded into four α-helical structure units in tandem,
4 sextuplets of protomers, each being a different sequence folded into six α-helical structure units in tandem,
3 octuplets of protomers, each being a different sequence folded into eight α-helical structure units in tandem,
2 duodecaplets of protomers, each being a different sequence folded into 12 α-helical structure units in tandem,
or
1 protomer, having a sequence folded into 24 α-helical structure units in tandem.

In certain embodiments, an asymmetric cTRP comprises the formula:

—R¹—R²—R³—R⁴—,
wherein R¹, R², R³, R⁴ are protomers, and each of R¹, R², R³, R⁴ (if present) are distinct from each other, the protein is left-handed; and the N- and C-termini of the protein created a circular architecture.

In certain embodiments,
R¹ is cTRP24(6)SS_aSym1 (SEQ ID NO:1),
R² is cTRP24(6)SS_aSym2 (SEQ ID NO:2),
R³ is cTRP24(6)SS_aSym3 (SEQ ID NO:3), and
R⁴ is cTRP24(6)SS_aSym4 (SEQ ID NO:4).

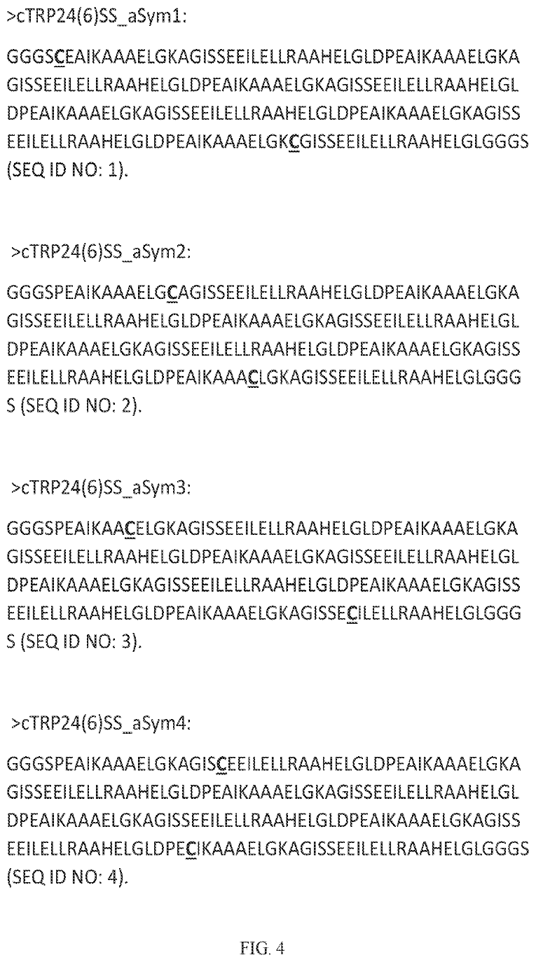

The following are exemplary protomers is the following (see FIG. 4): cTRP24(6)SS_aSym1 (SEQ ID NO:1), cTRP24(6)SS_aSym2 (SEQ ID NO:2), cTRP24(6)SS_aSym3 (SEQ ID NO:3), and cTRP24(6)SS_aSym4 (SEQ ID NO:4).

The protomer cTRP24(6)SS_aSym1 (SEQ ID NO: 1) consists of several α-helical structural units flanked by linkers in the following order:

(a)
(SEQ ID NO: 20)
GGGGSGGGGS,
(b)
(SEQ ID NO: 6)
CEAIKAAAELGKAGISSEEILELLRAAHELGLD,
(c)
(SEQ ID NO: 7)
PEAIKAAAELGKAGISSEEILELLRAAHELGLD,
(d)
(SEQ ID NO: 7)
PEAIKAAAELGKAGISSEEILELLRAAHELGLD,
(e)
(SEQ ID NO: 7)
PEAIKAAAELGKAGISSEEILELLRAAHELGLD,
(f)
(SEQ ID NO: 7)
PEAIKAAAELGKAGISSEEILELLRAAHELGLD,
(g)
(SEQ ID NO: 8)
PEAIKAAAELGKCGISSEEILELLRAAHELGL,
and
(h)
(SEQ ID NO: 5)
GGGS.

(Alternatively, the first residue G of the three contiguous glycine in (h) may be considered as the last residue in (g), thereby (g) having 33 amino acid residues as the other repeats (b)-(f), and (h) would be two contiguous glycine as in GGS instead.)

The protomer cTRP24(6)SS_aSym2 (SEQ ID NO: 2) consists of several α-helical structural units flanked by linkers in the following order:

(a)
(SEQ ID NO: 20)
GGGGSGGGGS,
(b)
(SEQ ID NO: 9)
PEAIKAAAELGCAGISSEEILELLRAAHELGLD,
(c)
(SEQ ID NO: 7)
PEAIKAAAELGKAGISSEEILELLRAAHELGLD,
(d)
(SEQ ID NO: 7)
PEAIKAAAELGKAGISSEEILELLRAAHELGLD,
(e)
(SEQ ID NO: 7)
PEAIKAAAELGKAGISSEEILELLRAAHELGLD,
(f)
(SEQ ID NO: 7)
PEAIKAAAELGKAGISSEEILELLRAAHELGLD,
(g)
(SEQ ID NO: 10)
PEAIKAAACLGKAGISSEEILELLRAAHELGL,
and
(h)
(SEQ ID NO: 5)
GGGS

(Alternatively, the first residue G of the three contiguous glycine in (h) may be considered as the last residue in (g), thereby (g) having 33 amino acid residues as the other repeats (b)-(f), and (h) would be two contiguous glycine as in GGS instead.)

The protomer cTRP24(6)SS_aSym3 (SEQ ID NO: 3) consists of several α-helical structural units flanked by linkers in the following order:

(a)
(SEQ ID NO: 20)
GGGGSGGGGS,
(b)
(SEQ ID NO: 11)
PEAIKAACELGKAGISSEEILELLRAAHELGLD,
(c)
(SEQ ID NO: 7)
PEAIKAAAELGKAGISSEEILELLRAAHELGLD,
(d)
(SEQ ID NO: 7)
PEAIKAAAELGKAGISSEEILELLRAAHELGLD,
(e)
(SEQ ID NO: 7)
PEAIKAAAELGKAGISSEEILELLRAAHELGLD,
(f)
(SEQ ID NO: 7)
PEAIKAAAELGKAGISSEEILELLRAAHELGLD,
(g)
(SEQ ID NO: 12)
PEAIKAAAELGKAGISSECILELLRAAHELGL,
and
(h)
(SEQ ID NO: 5)
GGGS

(Alternatively, the first residue G of the three contiguous glycine in (h) may be considered as the last residue in (g), thereby (g) having 33 amino acid residues as the other repeats (b)-(f), and (h) would be two contiguous glycine as in GGS instead.)

The protomer cTRP24(6)SS_aSym4 (SEQ ID NO: 4) consists of several α-helical structural units flanked by linkers in the following order:

(a)
(SEQ ID NO: 20)
GGGGSGGGGS,
(b)
(SEQ ID NO: 13)
PEAIKAAAELGKAGISCEEILELLRAAHELGLD,
(c)
(SEQ ID NO: 7)
PEAIKAAAELGKAGISSEEILELLRAAHELGLD,
(d)
(SEQ ID NO: 7)
PEAIKAAAELGKAGISSEEILELLRAAHELGLD,
(e)
(SEQ ID NO: 7)
PEAIKAAAELGKAGISSEEILELLRAAHELGLD,
(f)
(SEQ ID NO: 7)
PEAIKAAAELGKAGISSEEILELLRAAHELGLD,
(g)
(SEQ ID NO: 14)
PECIKAAAELGKAGISSEEILELLRAAHELGL,
and
(h)
(SEQ ID NO: 5)
GGGS

(Alternatively, the first residue G of the three contiguous glycine in (h) may be considered as the last residue in (g), thereby (g) having 33 amino acid residues as the other repeats (b)-(f), and (h) would be two contiguous glycine as in GGS instead.)

In the sequences, the position of cysteine mutations facilitating defined 1:1:1:1 hetero-complexes are indicated with a bolded/underlined letter “C.” In FIG. 4, the amino acid repeat units are color-coded in each protomer sequence to denote the unique interfaces of each protomer, with like-colored regions pairing with each other. For example, the α-helical structural unit consisting of the amino acids of SEQ ID NO:6 in cTRP24(6)SS_aSym1 (SEQ ID NO:1) pairs with the α-helical structural unit consisting of the amino acids of SEQ ID NO:14 in protomer cTRP24(6)SS_aSym4 (SEQ ID NO:4); the α-helical structural unit consisting of the amino acids of SEQ ID NO:8 in cTRP24(6)SS_aSym1 (SEQ ID NO:1) pairs with the α-helical structural unit consisting of the amino acids of SEQ ID NO:9 in protomer cTRP24(6)SS_aSym2 (SEQ ID NO:2); the α-helical structural unit consisting of the amino acids of SEQ ID NO:10 in cTRP24(6)SS_aSym2 (SEQ ID NO:2) pairs with the α-helical structural unit consisting of the amino acids of SEQ ID NO:11 in protomer cTRP24(6)SS_aSym3 (SEQ ID NO:3); and the α-helical structural unit consisting of the amino acids of SEQ ID NO:12 in cTRP24(6)SS_aSym3 (SEQ ID NO:3) pairs with the α-helical structural unit consisting of the amino acids of SEQ ID NO: 13 in protomer cTRP24(6)SS_aSym4 (SEQ ID NO:4).

In certain embodiments, the terminal α-helical structure unit has 90% identity to SEQ ID NO: 7. In certain embodiments, the terminal α-helical structure unit has 95% identity to SEQ ID NO: 7. In certain embodiments, the terminal α-helical structure has one cysteine substitution or one non-canonical amino acid substitution compared to SEQ ID NO:7. In certain embodiments, the C-terminal α-helical structure has one cysteine substitution or one non-canonical amino acid substitution compared to SEQ ID NO:7, and has the last amino acid residue different or truncated compared to SEQ ID NO:7. As will be understood by one of ordinary skill in the art, variants of the cTRP sequences, that do not alter the circular, handed and repetitive structural nature of the proteins can also be used. Indeed, variants of all protein sequences disclosed herein can be used, so long as the variation does not render the protein unfit for its intended purpose.

### Positioning of Cysteine or Noncanonical Amino Acid Mutations

Positioning of the cysteine residues or the noncanonical amino acids (with click chemistry reaction groups) in the repeat unit (aka α-helical structural unit) is important. Herein, the inventors have used in silico models, and neighboring interchain residues less than 3 angstroms apart were mutated to cysteines and refolded with AlphaFold2. Cysteines were placed where the interfaces of the protomers interact (i.e., via terminal repeats) so that they self-assemble to form disulfide bonds. Noncanonical amino acids with click chemistry reaction groups may also be placed in place of native amino acids, to facilitate click bonding at interfaces of protomers. Preferably, in the case of the asymmetric cTRPs, the disulfides or click reaction pairs were uniquely positioned at certain interfaces so that disulfide bonds or conjugations are formed at the top or the bottom of the interfaces.

The interface between individual helix—turn—helix repeats may be able to harbor multiple cysteine mutations or noncanonical amino acid mutations to facilitate hetero-oligomerization. In various embodiments, each mutation set is within 5.4 Å between alpha carbons to allow disulfide formation. More possible positions within 7 Å are defined in FIG. 9 (right panel). Each position number is identified as the position in an individual repeat and its nearest neighbor repeat which would need to be linked to achieve hetero-oligomerization. Each amino acid numbering is based on amino acid positions in a sequence having the amino acid sequence of PEAIKAAAELGKA (SEQ ID NO:15)—GBB linker—SEEILELLRAAHEL (SEQ ID NO:16)—GBB linker, or based on positions in a sequence aligned with the sequence having the amino acid sequence of PEAIKAAAELGKA (SEQ ID NO:15)—GBB linker—SEEILELLRAAHEL (SEQ ID NO:16)—GLD. Due to the repeating characteristics of α-helical structural units, the sequence of an α-helical structural unit can “begin” or “end” at different residues, per user designation. For instance, an α-helical peptide repeat unit may have different designations of a same sequence:

PEAIKAAAELGKA (SEQ ID NO: 15)-GBB linker-
SEEILELLRAAHEL (SEQ ID NO: 16) -GLD,
D-PEAIKAAAELGKA (SEQ ID NO: 15)-GBB linker-
SEEILELLRAAHEL (SEQ ID NO: 16)-GL,
LD-PEAIKAAAELGKA (SEQ ID NO: 15)-GBB linker-
SEEILELLRAAHEL(SEQ ID NO: 16)-G,
or
GLD-PEAIKAAAELGKA (SEQ ID NO: 15)-GBB linker-
SEEILELLRAAHEL(SEQ ID NO: 16).

Therefore, positioning of desirable substitutions is provided based on amino acid positions in PEAIKAAAELGKA (SEQ ID NO:15)—GBB linker—SEEILELLRAAHEL (SEQ ID NO: 16)—GLD, or when aligned with (and positions renumbered based on) PEAIKAAAELGKA (SEQ ID NO:15)—GBB linker—SEEILELLRAAHEL (SEQ ID NO:16)—GLD.

In various embodiments, positions of mutations for bonding between two terminal α-helical structural units (i.e., terminal repeats) of two protomers, also referred to as pair-wise positions, include:
position 1 and position 33,
position 1 and position 2,
position 1 and position 3,
position 1 and position 32,
position 2 and position 33,
position 3 and position 33,
position 3 and position 32,
position 4 and position 3,
position 4 and position 6,
position 12 and position 6,
position 8 and position 9,
position 8 and position 10,
position 8 and position 13,
position 8 and position 6,
position 11 and position 10,
position 11 and position 13,
position 11 and position 15,
position 12 and position 13,
position 17 and position 19,
position 21 and position 23,
position 28 and position 30,
position 4 and position 27,
position 11 and position 15, and
position 4 and position 33,
based on amino acid positions in PEAIKAAAELGKA (SEQ ID NO:15)—GBB linker—SEEILELLRAAHEL (SEQ ID NO:16)—GBB linker or in a sequence aligned therewith.

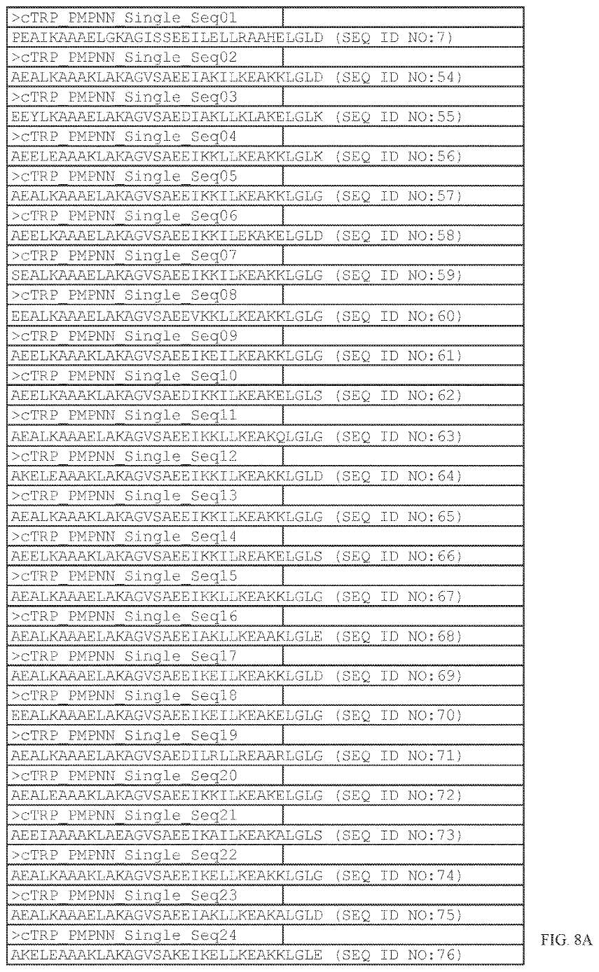

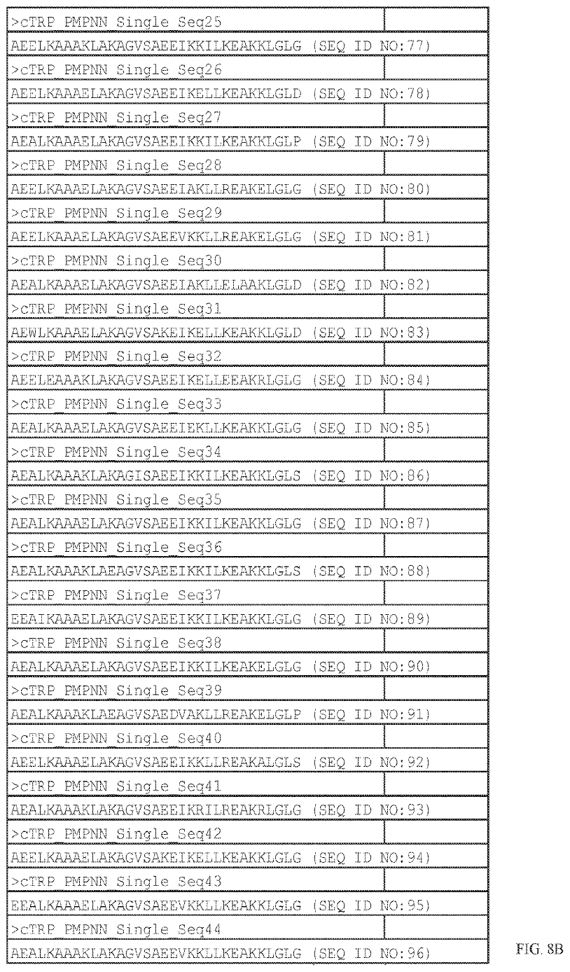

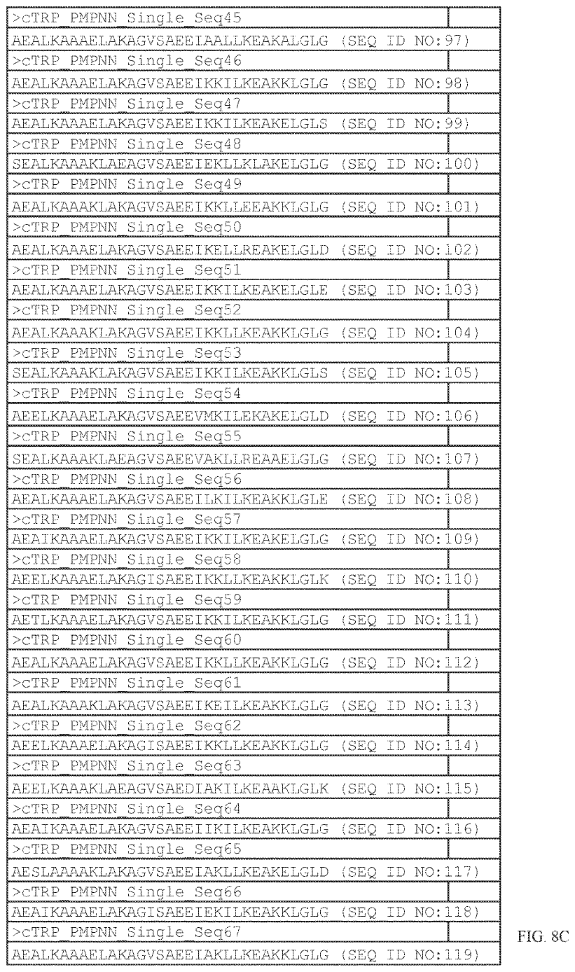

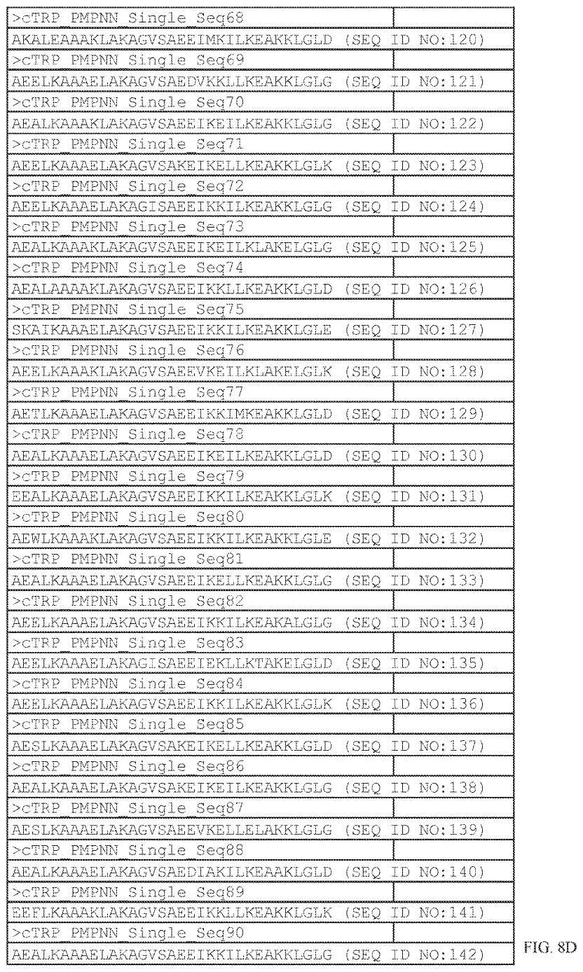

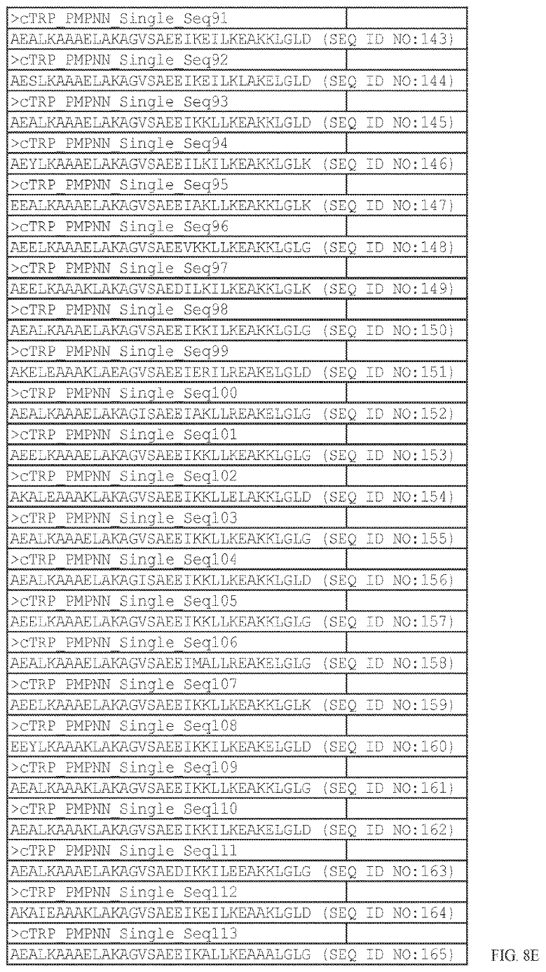

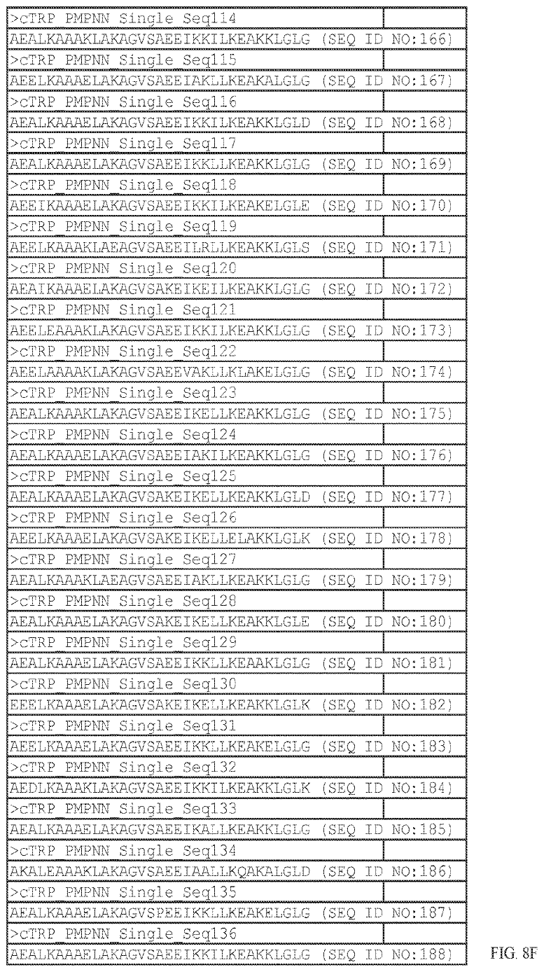

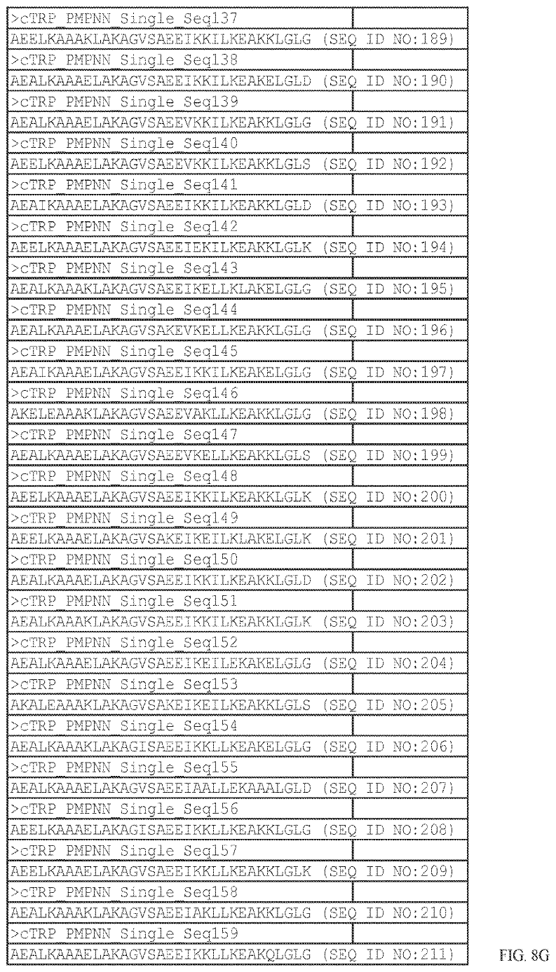

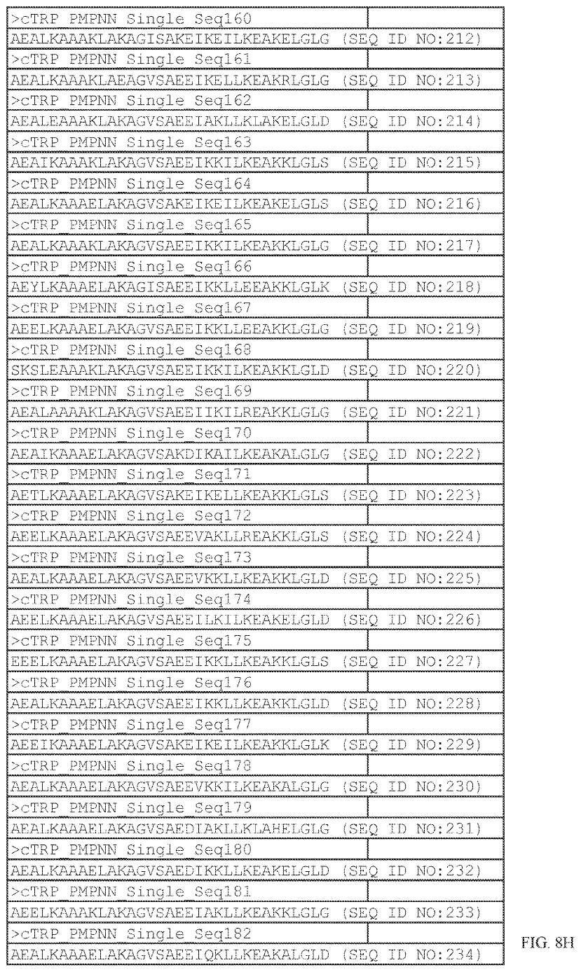

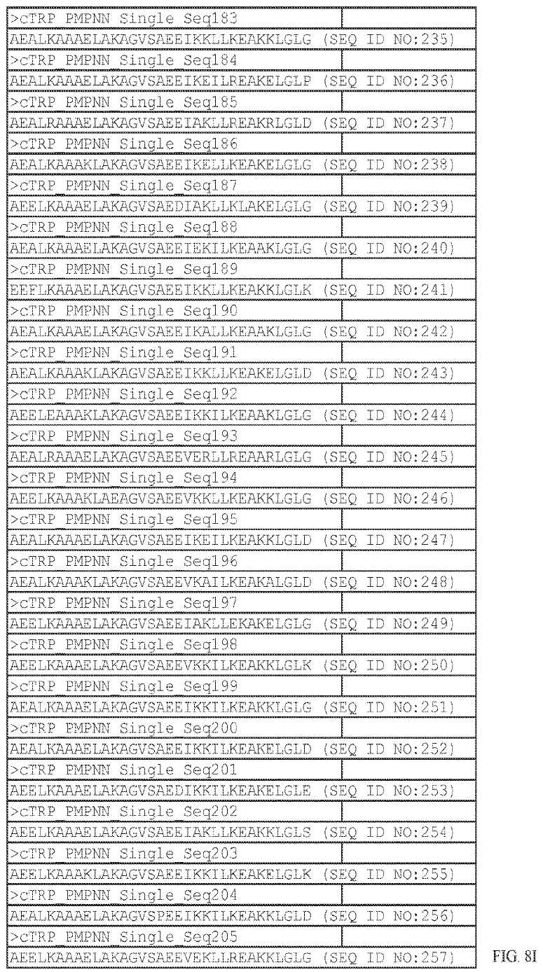

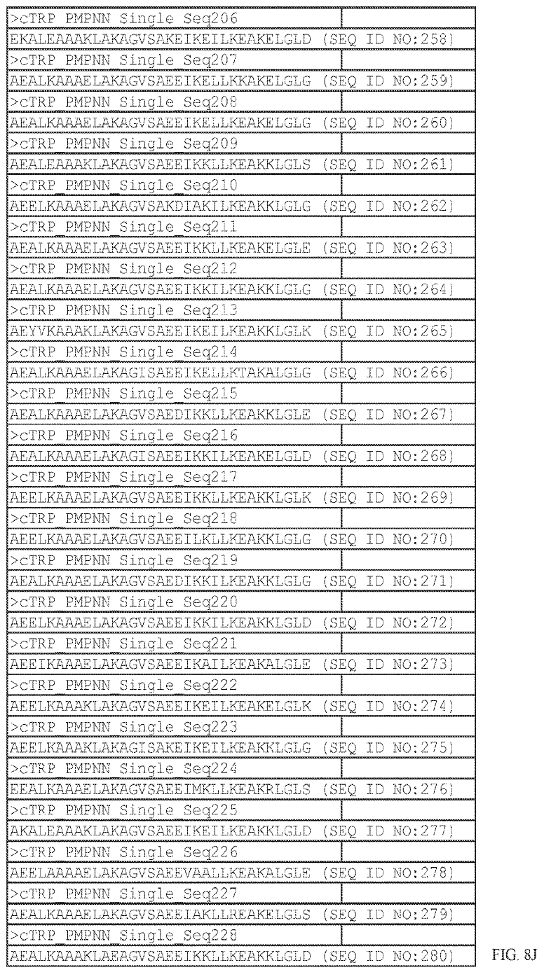

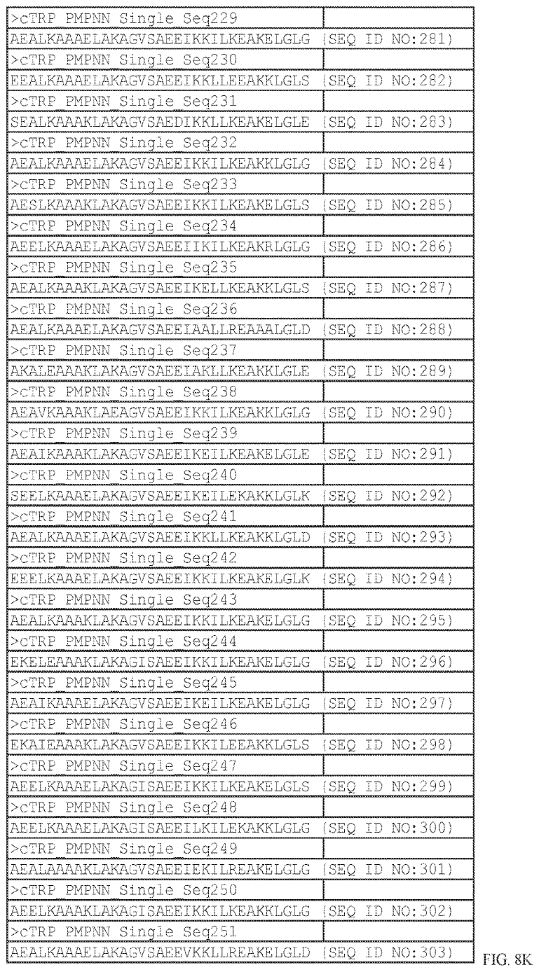

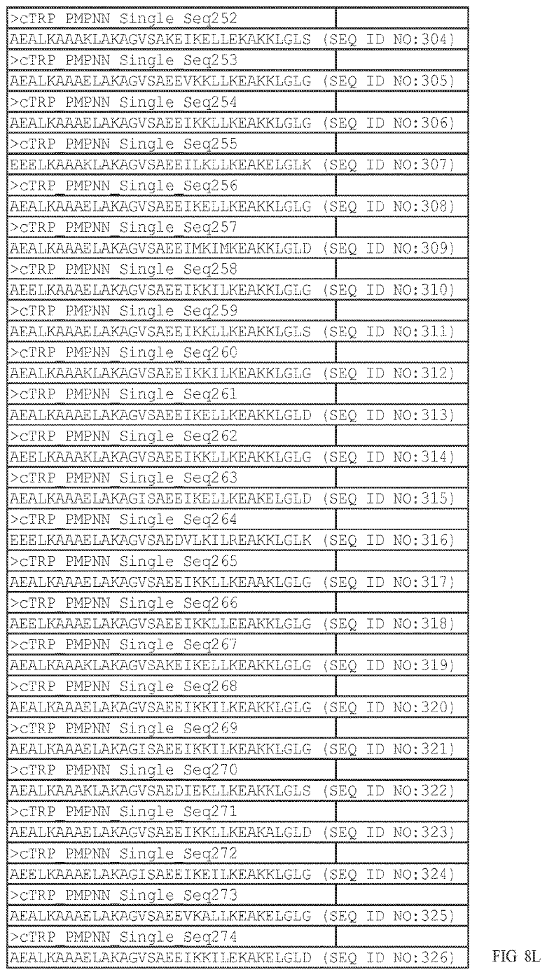

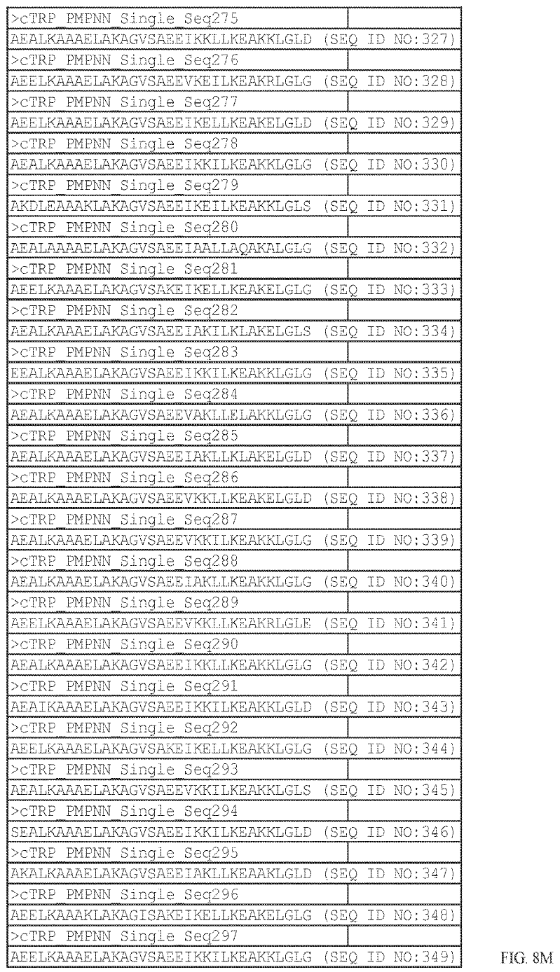

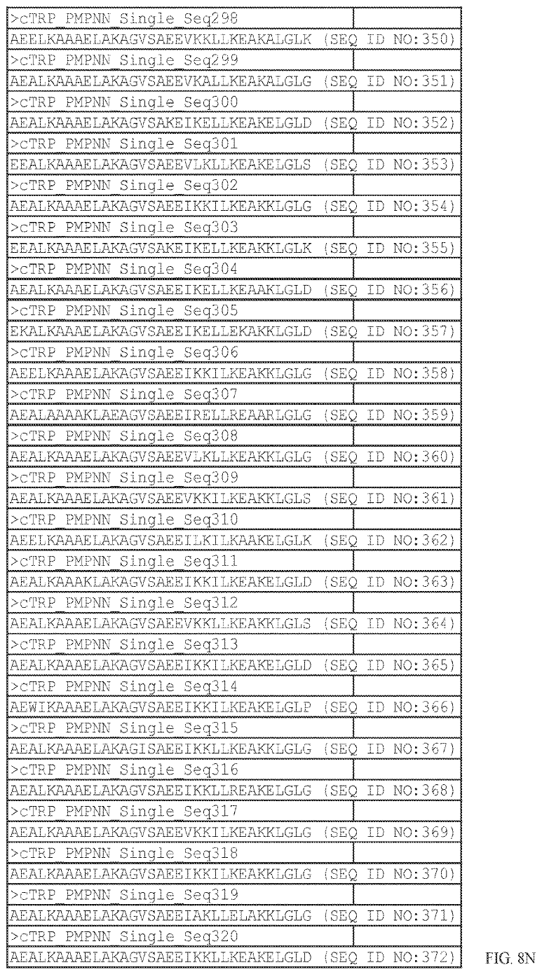

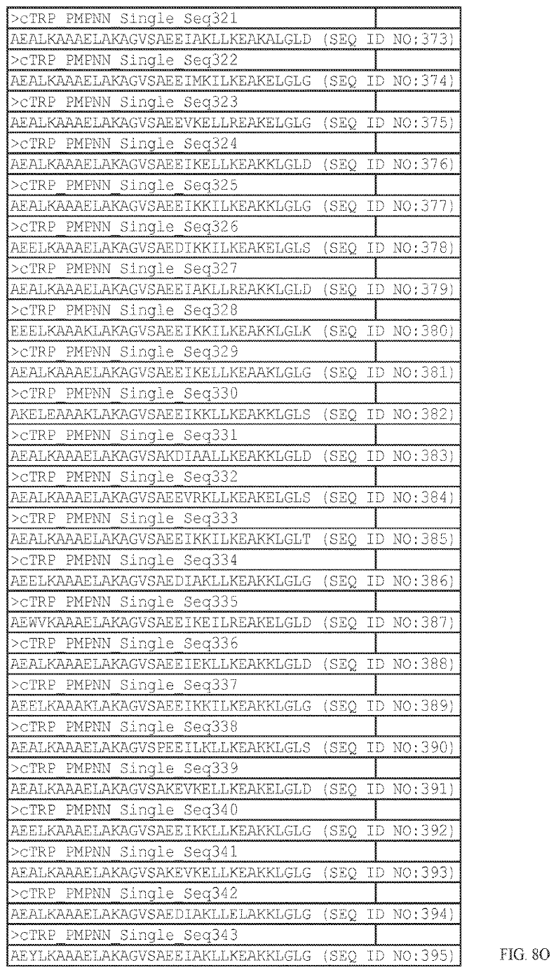

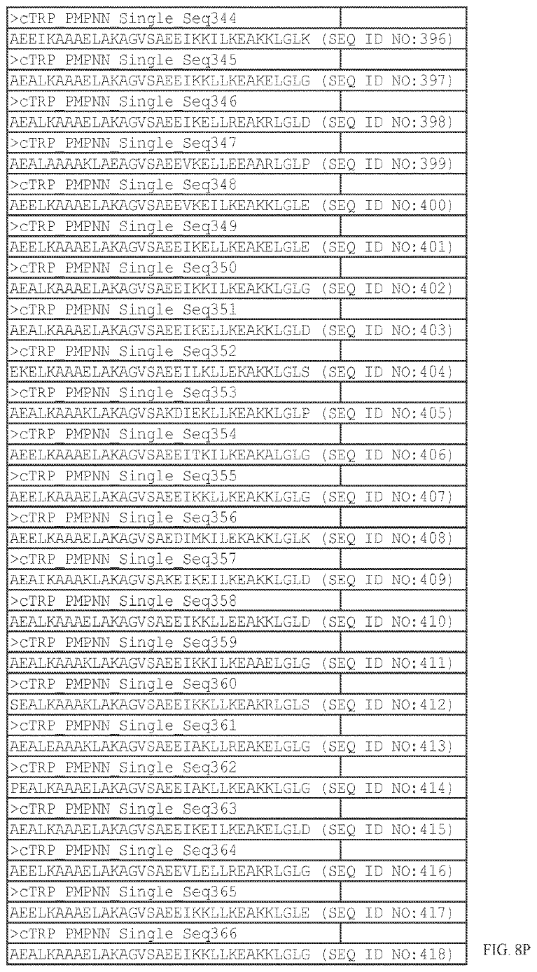

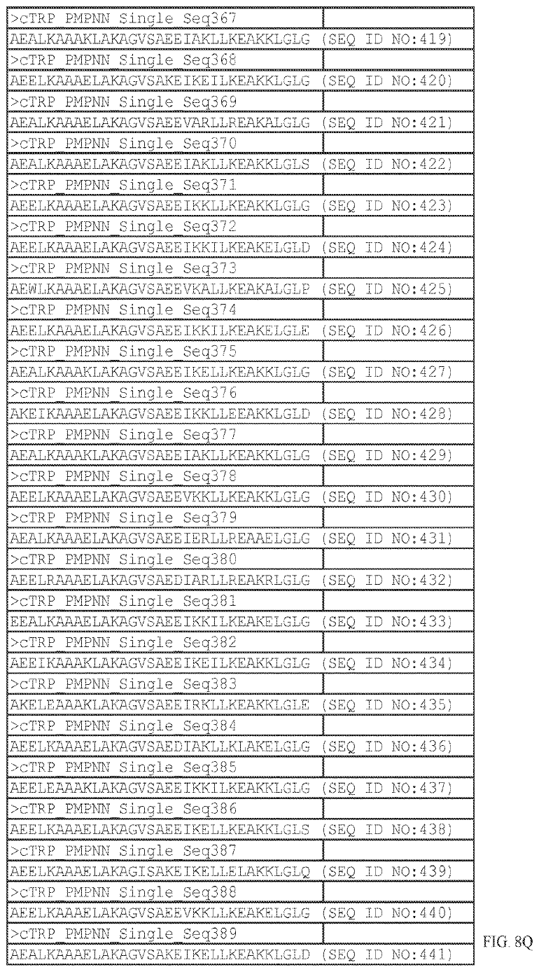

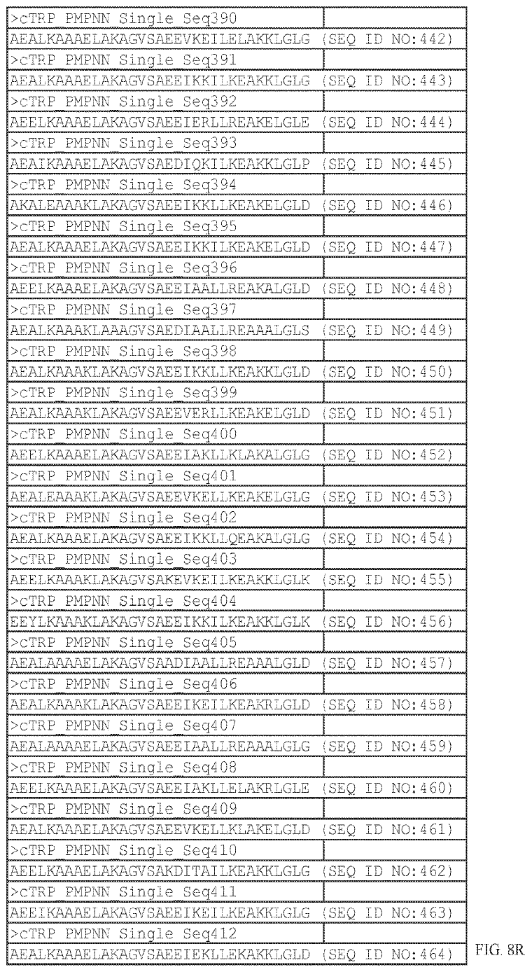

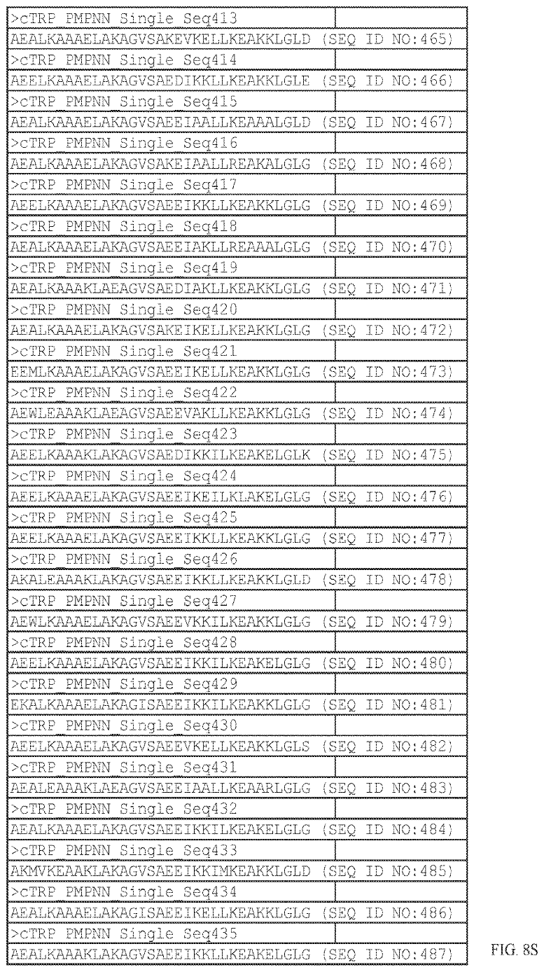

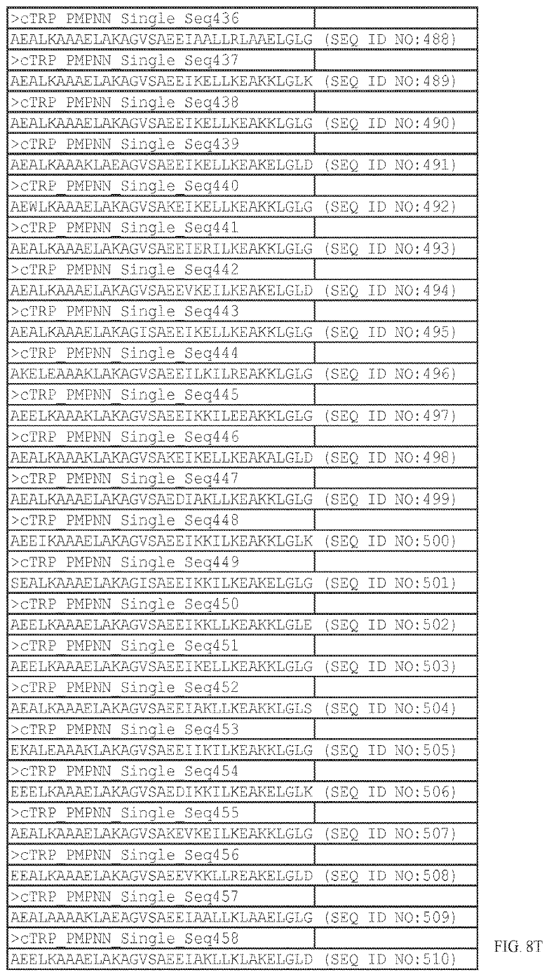

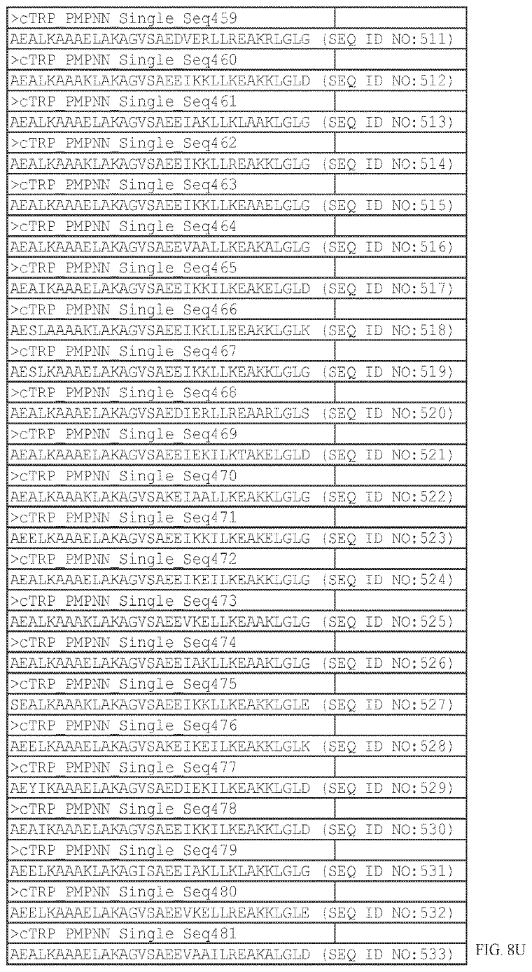

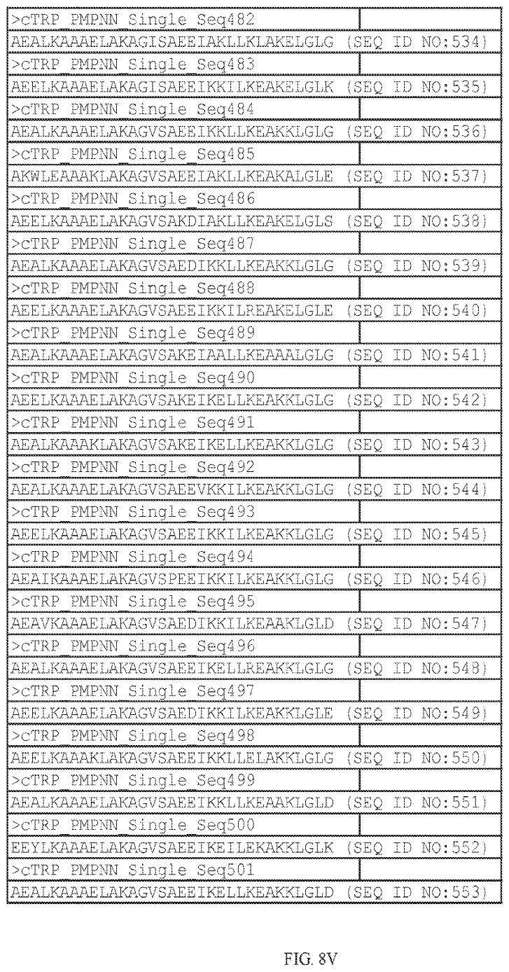

For example, amino acid position 1 of an α-helical structure unit (“repeat A”) is within 7 Å of positions 33, 2, 3, and 32 of another α-helical structure unit (“repeat B”), wherein the positions are based on amino acid positions in/aligned to PEAIKAAAELGKA (SEQ ID NO:15)—GBB linker—SEEILELLRAAHEL (SEQ ID NO:16)—GBB linker. For repeats having the amino acid sequence of PEAIKAAAELGKA (SEQ ID NO:15)—GBB linker—SEEILELLRAAHEL (SEQ ID NO:16)—GBB linker (such as cTRP_PMPNN_Single_Seq01 in FIG. 8A), amino acid position 1 is proline (hence P1), position 2 is glutamic acid (hence E2), position 3 is alanine (hence A3), and position 32 is leucine (hence L32). For repeats having the amino acid sequence of SEQ ID NO:54 (cTRP_PMPNN_Single_Seq02 in FIG. 8A), amino acid position 1 is alanine (hence a substitution with cysteine would be A1C).

As another example, amino acid position 2 of an α-helical structure unit (“repeat A”) is within 7 Å of position 33 of another α-helical structure unit (“repeat B”). For repeats having the amino acid sequence of PEAIKAAAELGKA (SEQ ID NO:15)—GBB linker—SEEILELLRAAHEL (SEQ ID NO:16)—GBB as linker (such cTRP_PMPNN_Single_Seq01 in FIG. 8A), amino acid position 2 is glutamic acid (hence E2), and amino acid position 33 is aspartic acid (hence D33). For repeats having the amino acid sequence of SEQ ID NO:64 (cTRP_PMPNN_Single_Seq12 in FIG. 8A), amino acid position 2 is lysine (hence a substitution with cysteine would be K2C), and amino acid position 33 is aspartic acid (hence a substitution with cysteine would be D33C).

Repeats A and B, in the left panel of FIG. 9, denotes two terminal repeats of two protomers, e.g., the C-terminal repeat of a prior protomer (“Repeat B”) and the N-terminal repeat of a next protomer (“Repeat A”) in a closed circular protein structure/cTRP; wherein the amino acid sequences of the repeats in the two protomers may otherwise be identical except for the amino acid substitutions and optionally further for the C-terminal amino acid deletion or substitution outside of the helix—turn—helix.

In various aspects, each interface (e.g., each pair including C-terminal repeat of a prior protomer and N-terminal repeat of a next protomer) of two or more protomers has a different pair-wise positions of mutations for introduction of disulfide bonding or conjugation between the protomers.

For example, a tetraoligomeric protein includes four protomers associated in tandem in a closed circular structure, wherein the four protomers have otherwise identical or different repeats of α-helical forming sequences except for substitutions in N- and C-terminal repeats, wherein the substitutions in the C-terminal repeat of any one of the four protomers and the N-terminal repeat of a next protomer in tandem are at amino acid positions selected from positions 1 and 3, positions 8 and 9, positions 12 and 13, and positions 17 and 19, relative to respective terminal repeats, wherein the amino acid numbering is based on amino acid positions in PEAIKAAAELGKA (SEQ ID NO: 15)—GBB linker—SEEILELLRAAHEL (SEQ ID NO:16)—GBB linker or based on amino acid positions in a sequence having at least 95%, 90%, 85%, 80%, 75%, 70%, 65%, 60%, 55%, or 50% identity to and being aligned with PEAIKAAAELGKA (SEQ ID NO:15)—GBB linker—SEEILELLRAAHEL (SEQ ID NO:16)—GBB linker.

In some embodiments, a protomer does not include amino acid substitutions at terminal repeats at positions 1 and 3, based on amino acid positions in PEAIKAAAELGKA (SEQ ID NO: 15)—“GBB” type linker—SEEILELLRAAHEL (SEQ ID NO:16)—GLD or based on amino acid positions in a sequence aligned with PEAIKAAAELGKA (SEQ ID NO:15)—“GBB” type linker—SEEILELLRAAHEL (SEQ ID NO:16)—GLD. In some embodiments, Y and Z of a protomer do not have the substitution with cysteine or with non-canonical amino acid at positions 1 and 3, (e.g., neither position 1 in Y and position 3 in Z, nor position 3 in Y and position 1 in Z), wherein Y and Z are each 30-35 amino acids long (e.g., 33 amino acids long) and comprising an α-helix—turn—α-helix structure, e.g., PEAIKAAAELGKA (SEQ ID NO:15)—“GBB” type linker—SEEILELLRAAHEL (SEQ ID NO:16).

Further embodiments provide that an oligomeric protein comprising two or more protomers associated in tandem in a closed circular structure, wherein each protomer contains repeats of α-helical forming sequences selected from any one of those in FIGS. 8A-8V (SEQ ID NOs.: 7 and 54-553, wherein N- and C-terminal repeats of each protomer have amino acid substitutions with a cysteine or a noncanonical amino acid.

Properly designed protomers self-assemble into cTRPs as they move through the secretory pathway or in the supernatant of a cell culture. In addition to being circular, handed, and structurally repetitive, the cTRPs disclosed herein exhibit self-folding, high thermostability, and high solubility. Self-folding means that the cTRPs fold without any need for inclusion of additional folding domains or subunits (e.g., additional protein domains physically appended to the cTRP construct, or independently added protein folding chaperones such as GroEL/GroES or redox-dependent folding cofactors such as thioredoxin). High thermostability means that the proteins retain their overall secondary structure (including alpha-helices) and tertiary structures (defined by their size and shape) at temperatures as high as 95° C. High solubility means that the proteins can be concentrated to levels of 1 mg/mL or higher at physiological pH and salt concentrations without formation of soluble protein aggregates or protein precipitate.

The asymmetric cTRPs discussed herein have applications as scaffolds for binding and catalysis and as building blocks for higher-order assemblies. Amino acids lining the central pores can be mutated to introduce binding or catalytic functionalities and/or sites of chemical modification. Monomeric toroids could replace multimeric assemblies as symmetry centers in the assembly of protein cages; by breaking the symmetry of the interaction surfaces it is possible to create more complex heterotypic assemblies with non-uniform placement of functional sites. Thus, the cTRPs disclosed herein can be exploited as insertion sites for peptides or protein moieties. The largest of the engineered repeat proteins created to date accommodates up to 24 inserted elements of variable composition. This provides highly adaptable nanoparticles for the display and delivery of precisely arranged arrays of peptides or protein factors. Particular embodiments can further include up to 50, 60, 70, 80, 90, 100, 110, 120, or 130 repetitive α-helical structures and/or functional domains of variable composition. Particular embodiments can include 96 or 124 repetitive α-helical structures and/or functional domains of variable composition that need to be cotransduced or added together in equimolar ratios to assemble into full cTRPs.

The protomers are co-transduced or added together in equimolar ratios to assemble into full cTRPs.

Use of Non-Canonical Amino Acids to Connect cTRP Protomers

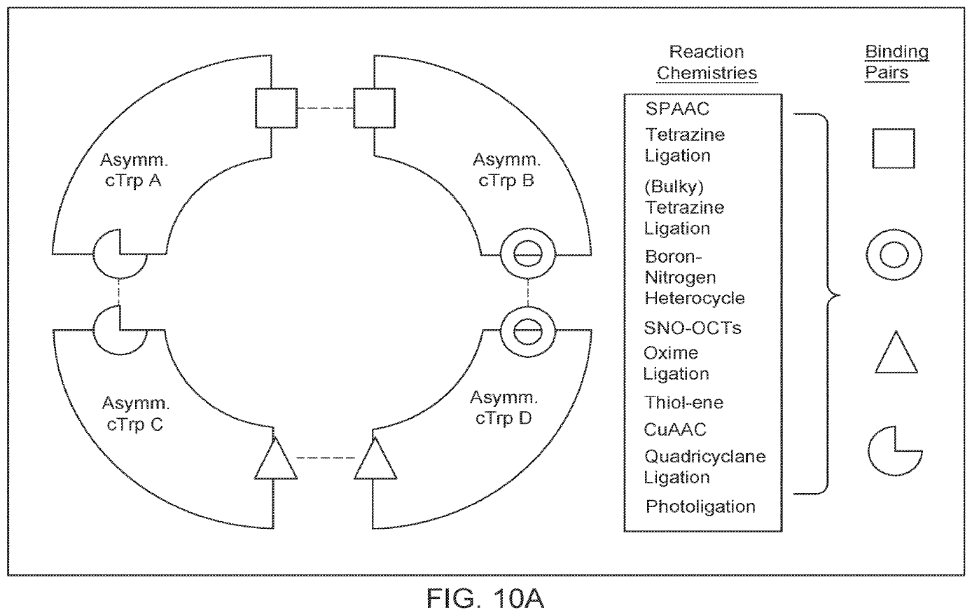

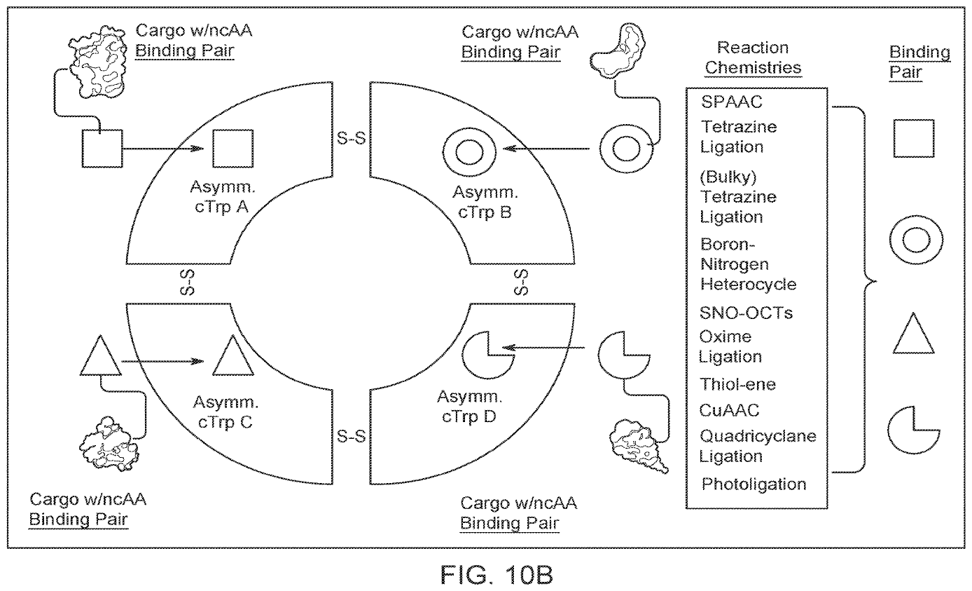

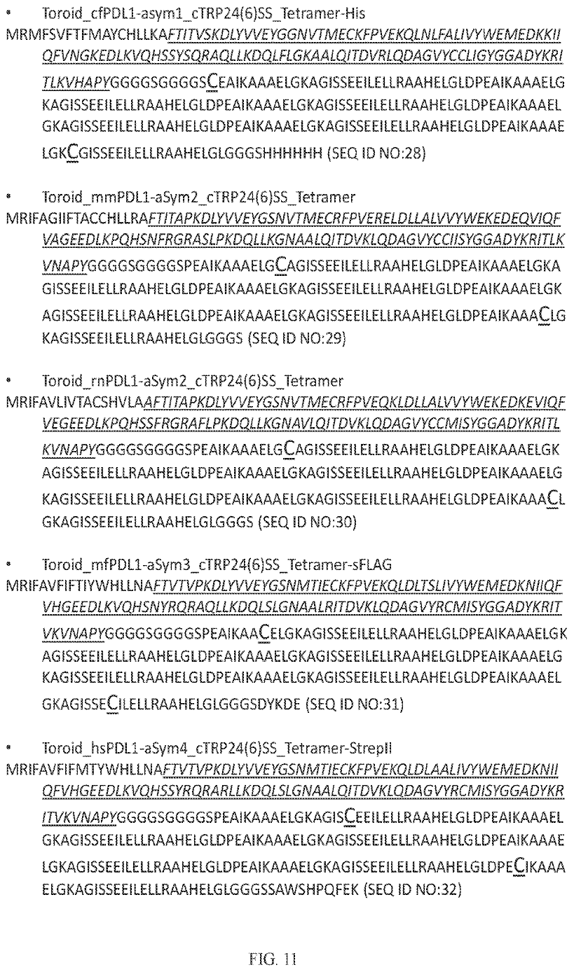

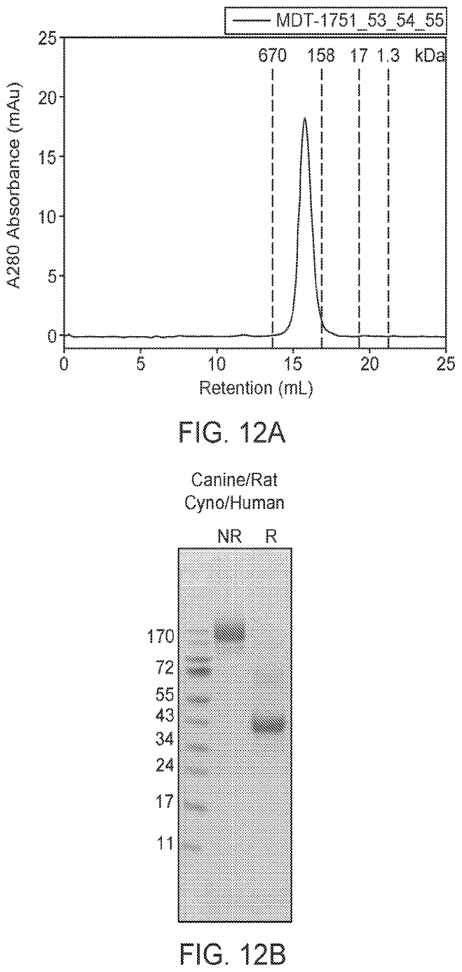

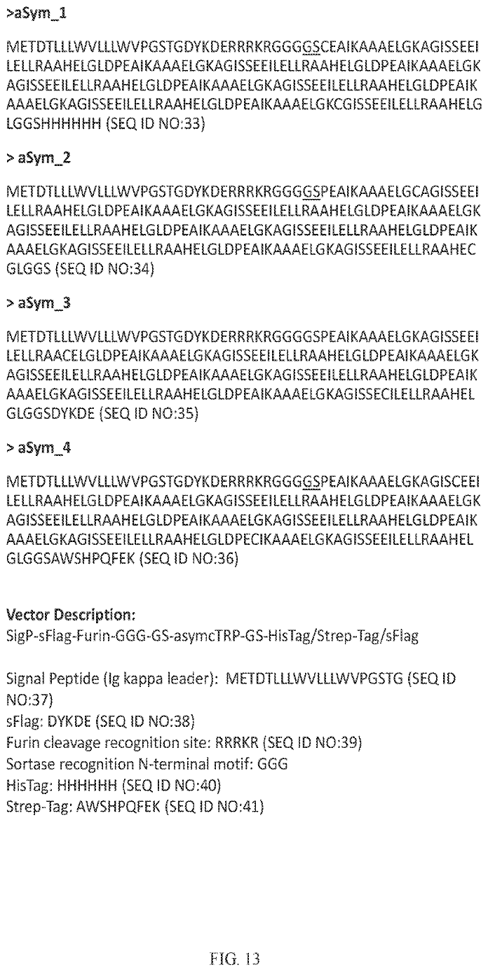

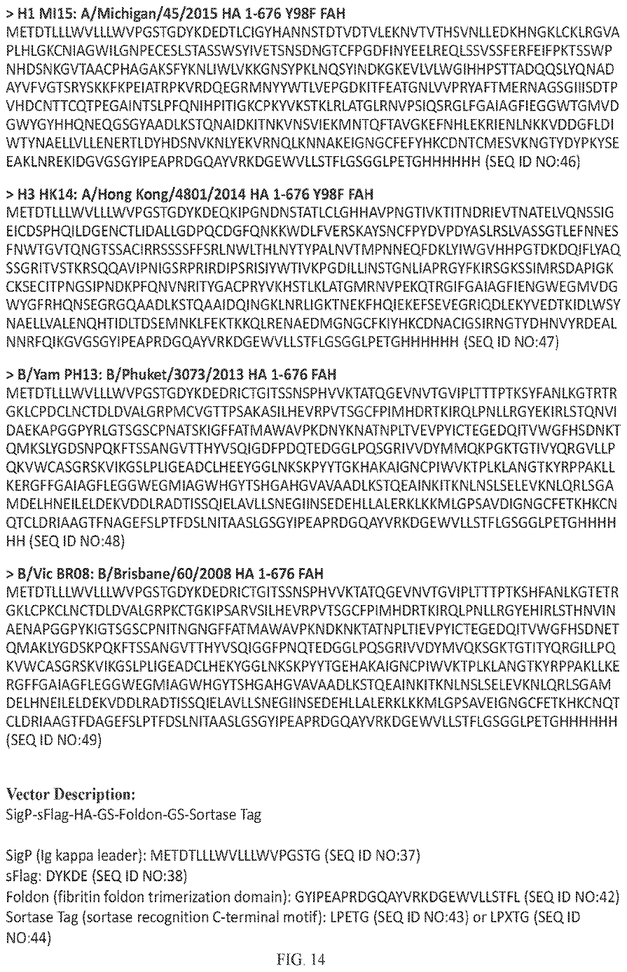

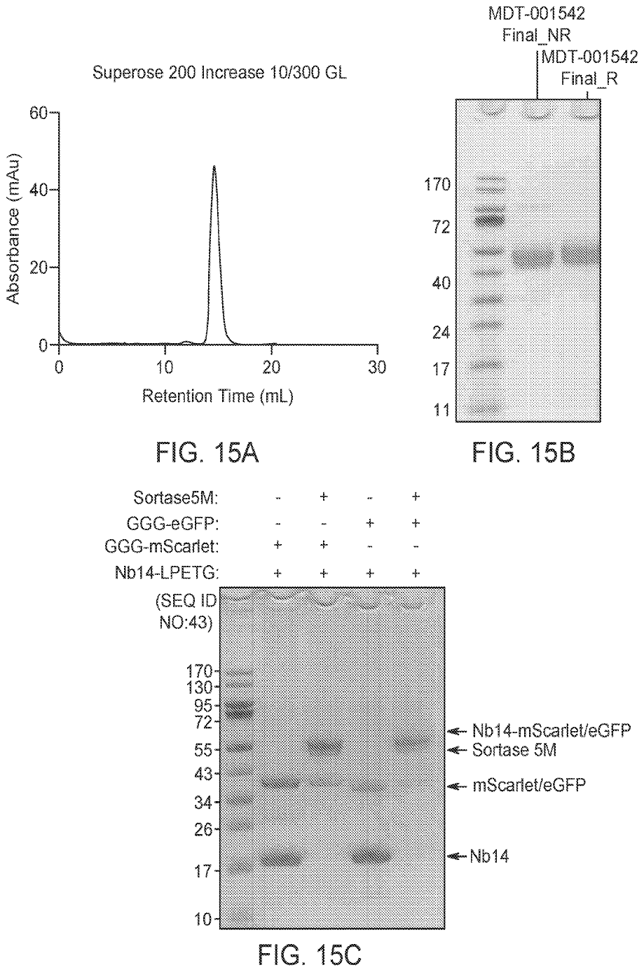

cTRP Protomers can be self-assembled asymmetrically through click chemistry without disulfide bonds. To link together four separate protomers, a combination of four mutually orthogonal binding pairs is required. To self-assemble the protomers, any four of these ten mutually orthogonal click chemistries can be utilized, including: SPAAC, Tetrazine ligation, Bulky Tetrazine-isonitrile ligation, Boron+Nitrogen Heterocycle formation, SnO Oct+Type I/II dipole, Oxime ligation, Thiol-ene ligation, CuACC, Quadricyclane ligation, and photoligation. See Yao, T. et al (2021) Molecules 26(17): 5368. The incorporation of ncAA pairs containing reactive handles to any four of these chemistries can be inserted at the cTRP interfaces to facilitate pairing. There are a total of 210 possible combinations. (See FIG. 10A)

In certain embodiments, “one-pot” reactions are performed with mutually orthogonal pairs at physiological pH. Not only are these reactions compatible with cells, they do not interference with each other.

Examples where no catalysts are needed:
1. Azide+Strained Alkyne (SPAAC)
2. Tetrazine-TCO (Tetrazine ligation)
3 (di)tert-butyl-tetrazine+isonitrile (Bulky Tetrazine ligation)
4. Boron+Nitrogen (Heterocycle formation)
5. SnO Oct+Type I/II dipole (SNO-OCT)

Examples where catalysts are needed a physiological pH:
6. Aldehyde+Alkoxyamine (Oxime)
7. Thiol+Alkene (Thiol-ene)
8. Azide+Alkene (CuACC)

Examples where light is needed to prepare reagent:
9 Quadricyclane+Strained alkene (Ni bis(dithiolene)) (Quadricyclane ligation)
10. Strained ring+alkyne (Photoligation)

It was found that up to 10 separate chemistries could be performed in one pot (210 possible combinations of 4 cTRp monomers).

cTRP Protomers can be self-assembled asymmetrically through click chemistry without disulfide bonds. To link together four separate protomers, a combination of four mutually orthogonal binding pairs is required. To self-assemble the protomers, any four of these ten mutally orthogonal click chemistries can be utilized, including: SPAAC, Tetrazine ligation, Bulky Tetrazine-isonitrile ligation, Boron+Nitrogen Heterocycle formation, SnO Oct+Type I/II dipole, Oxime ligation, Thiol-ene ligation, CuACC, Quadricyclane ligation, and photoligation. The incorporation of ncAA pairs containing reactive handles to any four of these chemistries can be inserted at the cTRP interfaces to facilitate pairing. There are a total of 210 possible combinations. FIG. 10A.

cTRP Protomers can also be decorated with cargo in an asymmetric manner using site-specific click chemistry and ncAA. ncAA with a complimentary chemical partner can be incorporated into the solvent-facing structure of each protomer. Then, proteinaceous cargo expressing ncAA with its own complimentary chemical partner can be conjugated to the desired protomer in a site-specific manner. FIG. 10B.

### Small Bioorthogonal Noncanonical Amino Acid Pairs

In various embodiments, N- or C-terminal repeat of a first protomer and C- or N-terminal repeat of a second protomer, (repeats at “opposing” terminus of two different protomers), each have an amino acid substitution at a selected position with a molecule from the A list or a molecule from the B list of a Reaction Pair below, thereby constituting a pair of amino acid substitutions at pair-wise positions relative to respective terminal repeat, as noted in FIG. 9 right panel, with molecules selected from one of a CuACC Reaction Pair, a Thiol-ene Reaction Pair, a SPAAC Reaction Pair, an Isonitrile-Tetrazine Reaction Pair, an Oxime Ligantion Reaction Pair. That is, a pair of amino acids on α-helical structural units at opposing terminal repeats of two different protomers are replaced with molecules of a “click” reaction pair, thereby facilitating the “click” reaction between the two different protomers so as to bond them “in tandem” (a protomer's C-terminal repeat is bonded to another promoter's N-terminal repeat via the “click” reaction pair).

In various embodiments, to form an asymmetric circular tandem repeat protein (cTRP), two or more protomers each have an amino acid substitution on the N-terminal repeat and an amino acid the C-terminal repeat of a prior protomer and the N-terminal repeat of a next protomer—in a circular tandem sequence substitution on the C-terminal repeat, wherein (1) the amino acid substitutions on the C-terminal repeat of a prior protomer and the N-terminal repeat of a next protomer—in a circular tandem sequence—are a pair made with molecules of a “click” reaction pair, and (2) every different amino acid substitution pairs are made with “click” reaction pair molecules of a different “click” reaction category, i.e., orthogonal reaction pairs, and optionally (3) every different amino acid substitution pairs are at different pair-wise positions noted in FIG. 9 right panel.

In some embodiments, a protein (or asymmetric cTRP) is provided, which include two or more protomers associated in a closed circular structure, each protomer having an amino acid substitution on each terminal repeat, wherein (1) the amino acid substitutions on the C-terminal repeat of a prior protomer and the N-terminal repeat of a next protomer—in the circular tandem sequence—are a pair made with molecules of a “click” reaction pair, and wherein (2) different amino acid substitution pairs are made with “click” reaction pair molecules of a different “click” reaction category. In embodiments wherein different amino acid substitution pairs are made with reaction pair molecules of a different “click” reaction category, the position of amino acid substitution for each pair may be different, or the same.

In some embodiments, a protein (or asymmetric cTRP) is provided, which include two or more protomers, each having an amino acid substitution on each terminal repeat, wherein (1) the amino acid substitutions on the C-terminal repeat of a prior protomer and the N-terminal repeat of a next protomer—in a circular tandem sequence—are a pair made with molecules of a “click” reaction pair, wherein (2) different amino acid substitution pairs are made with “click” reaction pair molecules of a different “click” reaction category, and (3) different amino acid substitution pairs are at different positions relative to respective terminal repeat selected from those pair-wise positions in FIG. 9 right panel.

Exemplary different “click” reaction categories include CuACC reaction, thiol-ene reaction, SPAAC reaction, isonitrile-tetrazine reaction, and oxime ligation reaction.

Molecules of a “click” reaction pair include one member A and one member B under one “click” reaction category. For example, a CuACC reaction pair can be (S)-2-amino-3-azido-propionic acid and (S)-2-aminopent-4-ynoic acid; (S)-2-amino-3-azido-propionic acid and (S)-2-aminohex-5-ynoic acid; (S)-2-amino-4-azido-butanoic acid and (S)-2-aminopent-4-ynoic acid; or (S)-2-amino-4-azido-butanoic acid and (S)-2-aminohex-5-ynoic acid.

### CuACC Reaction Pairs

A. (S)-2-amino-3-azido-propionic acid or (S)-2-amino-4-azido-butanoic acid
B. (S)-2-aminopent-4-ynoic acid or (S)-2-aminohex-5-ynoic acid

### Thiol-Ene Reaction Pairs

A. (S)-2-aminobut-3-enoic acid or (S)-2-aminopro-4-enoic acid
B. 2-amino-3-mercaptopropanoic acid (L-Cysteine)

### SPAAC Reaction Pairs

A. (S)-2-amino-3-azido-propionic acid or (S) 2-amino-4-azido-butanoic acid
B. (S)-2-amino-2-((1R, 8S, 9r)-bicyclo[6.1.0]non-4-yn-9-yl) acetic acid

### Isonitrile-Tetrazine Reaction Pairs

A. (S)-2-amino-3-cyanopropanoic acid or (S)-2-amino-4-cyanobutanoic acid
B. (S)-2-amino-3-(4-(6-methyl-1,2,4,5-tetrazin-3-yl)phenyl) propanoic acid

### Oxime Ligation Reaction Pairs

A. (S)-2-(N-tert-Butoxycarbonyl)-4-[N-[2-chlorobenzyloxycarbonyl]-N-methoxy]diaminobutanoic acid
B. (S)-2-amino-3-(3-formylphenyl) propanoic acid

### Linkers That Attach Cargo Molecules to the Protomers

In embodiments incorporating functional domains, linkers can be used between the scaffold cTRP sequence and the functional domain sequence. Linkers can be used that fuse domains together and result in stably expressed, functional proteins. Examples of linkers can be found in Chen et al., Adv Drug Deliv Rev. 2013 Oct. 15; 65(10): 1357-1369. Linkers can be flexible, rigid, or semi-rigid, depending on the desired functional domain presentation to a target. In some embodiments, the linker is an in vivo cleavable linker (see, e.g., WO 2023/044321 A1). Commonly used flexible linkers include Gly-Ser linkers such as GGSGGGSGGSG (SEQ ID NO: 17), GGSGGGSGSG (SEQ ID NO:18) and GGSGGGSG (SEQ ID NO:19). Additional examples include: GGGGSGGGGS (SEQ ID NO:20); GGGSGGGS (SEQ ID NO:21); and GGSGGS (SEQ ID NO:22).

In some embodiments, flexible linkers may be incapable of maintaining a distance or positioning of functional domains needed for a particular use. In these instances, rigid or semi-rigid linkers may be useful. Examples of rigid or semi-rigid linkers include proline-rich linkers. In particular embodiments, a proline-rich linker is a peptide sequence having more proline residues than would be expected based on chance alone. In particular embodiments, a proline-rich linker is one having at least 30%, at least 35%, at least 36%, at least 39%, at least 40%, at least 48%, at least 50%, or at least 51% proline residues. Particular examples of proline-rich linkers include fragments of proline-rich salivary proteins (PRPs).

The rigidity of protein linkers refers to the degree of flexibility of the protein backbone over the entire length of a short, single chain protein as measured by the average root-mean-square (RMS) (RMS^{fluct}) of all internal torsion angles (φ,ψ) over the length of a given single chain protein linker.

RMS^{fluc} of a torsion angle is the standard deviation of the torsion angle value about the time-averaged value in a CHARMm molecular dynamics simulation, wherein RMS^{fluc} is calculated as follows:

$$R M {S}^{fluct} = \sqrt{\frac{1}{{N}_{f}} {\sum }_{f} {( {θ}^{f} - {θ}^{ave} )}^{2}}$$

where f refers to the frame number, N is the total number of frames in the trajectory file, and of and θ^{ave} are the current value and the average value for the torsion angle, respectively.

“CHARMm” (Chemistry at HARvard Macromolecular Mechanics) refers to a computer simulation engine (see Brooks et al., (1983) J Comp Chem 4:187-217; Mackerell, et al., (1998) J. Phys. Chem. B 102(18): 3586-3616; and “CHARMM: The Energy Function and Its Parameterization with an Overview of the Program.” by Mackerell et al., in The Encyclopedia of Computational Chemistry, Volume 1, 271-277, by Paul von Raque Schleyer et al., editors (John Wiley & Sons: Chichester, United Kingdom (1998)); and Brooks, et al., (2009) J. Comp. Chem., 30:1545-1615 (2009).

In particular embodiments, the average RMS^{fluc} can be calculated using the formula: (average RMS^{fluc} phi(φ)+average RMS^{fluc} psi(ψ))/2. The average RMS fluctuation of all internal backbone torsion angles over the length of the protein can be used to quantify the rigidity of the protein linker. The more rigid the protein is the smaller the average RMS fluctuation should be due to a more limited conformational space accessible to the protein.

In particular embodiments, a rigid protein linker refers to a linker having an average RMS^{fluc} of 25 or less, 20 or less 15 or less when measured using CHARMm modeling over a production run of 200 picoseconds (ps). In particular embodiments, a semi-rigid protein linker refers to a linker having an average RMS^{fluc} of 45-25 when measured using CHARMm modeling over a production run of 200 picoseconds (ps). In some embodiments cargo can be covalently conjugated using mammalian expressed enzyme, sortase. Where the recognition motif on the asymmetric cTRP can be either LPXTG (SEQ ID NO:44) (where X can be substitute with any amino acid) at the c-terminus or pentaglycine, triglycine, or diglycine motif on the N-terminus. The recognition motif on the functional domain can be either LPXTG (SEQ ID NO:44) at the c-terminus or pentaglycine, triglycine, or diglycine motif on the N-terminus.

cTRP protomers can also be decorated with cargo in an asymmetric manner using site-specific click chemistry and ncAA. ncAA with a complimentary chemical partner can be incorporated into the solvent-facing structure of each protomer. Then, proteinaceous cargo expressing ncAA with its own complimentary chemical partner can be conjugated to the desired protomer in a site-specific manner. (See FIG. 10B).

In certain embodiments, R¹ of a given protomer is an amino acid sequence tag that facilitates enzymatic attachment of protein cargo.

In certain embodiments, R² of a given protomer is an amino acid sequence tag that facilitates enzymatic attachment of protein cargo.

In certain embodiments, R¹ of a given protomer is an amino acid sequence GGG.

In certain embodiments, R² of a given protomer is a Sortag sequence LPXTG (SEQ ID NO: 44).

### Cargo Molecules

In particular embodiments cTRPs include one or more functional domain. Examples of functional domains that can be incorporated into cTRPs include peptide binding domains, phosphotyrosine binding domains, LIM domains, SAM domains, PDZ domains, FERM domains, Pleckstrin homology domains, VVVV domains, oligosaccharide binding (OB) domains, Immunoglobulin domains (IgG), single chain MHC proteins, tumor specific antigens (such as CD19 or ROR1), receptor ectodomains, isolated antigenic or adjuvant peptides, small molecule ligand binding domains (e.g., maltose binding protein) and enzyme domains (for example, trypsin). In particular embodiments the functional domains include SH2, Protein L, cytokines (e.g., IL-2; IL-17c, IL-3); Notch ligands (e.g., Delta or Jagged); immunogenic peptides; peptide adjuvants; nanobodies (Nbs), viral and bacterial immunogens, as well as individual members of catalytic pathways. In certain embodiments, the cargo is an antibody single chain variable fragment, a nanobody, and/or a viral, prokaryotic, or eukaryotic protein.

In certain embodiments, the cargo molecule is a T-cell engager, such as CD28, CD3, or CD70.

In certain embodiments, the cargo molecule is an ectodomain targeted by a chimeric antigen receptor (CAR), such as B7H3, HER2, EGFR, or IL17Ra. In certain embodiments, direct genetic fusion is utilized where a vector encodes a continuous polypeptide chain including the cargo, linker and cTRP protomer. In other instances, fusion may be achieved through a transpeptidase enzyme and inclusion of requisite tags on the proteins to be fused.

In certain embodiments, the cargo molecule is a microbe or a microbial subunit, a protein, or a small molecule ligand binding domain.

In certain embodiments, the cargo molecule is a microbe or microbial subunit.

In certain embodiments, the cargo molecule is a microbe, and the microbe is a virus.

In certain embodiments, the virus is influenza. Currently, there are 16 recognized hemagglutinins and nine neuraminidases, each of which has mutational variants. Examples of type A subtypes include, but are not limited to, H10N7, H10N1, H10N2, H10N3, H10N4, H10N5, H10N6, H10N7, H10N8, H10N9, H11N1, H11N2, H11N3, H11N4, H11N6, H11N8, H11N9, H12N1, H12N2, H12N4, H12N5, H12N6, H12N8, H12N9, H13N2, H13N3, H13N6, H13N9, H14N5, H14N6, H15N2, H15N8, H15N9, H16N3, H1N1, H1N2, H1N3, H1N5, H1N6, H1N8, H1N9, H2N1, H2N2, H2N3, H2N4, H2N5, H2N6, H2N7, H2N8, H2N9, H3N1, H3N2, H3N3, H3N4, H3N5, H3N6, H3N8, H3N9, H4N1, H4N2, H4N3, H4N4, H4N5, H4N6, H4N7, H4N8, H4N9, H5N1, H5N2, H5N3, H5N4, H5N6, H5N7, H5N8, H5N9, H6N1, H6N2, H6N3, H6N4, H6N5, H6N6, H6N7, H6N8, H6N9, H7N1, H7N2, H7N3, H7N4, H7N5, H7N7, H7N8, H7N9, H8N2, H8N4, H8N5, H9N1, H9N2, H9N3, H9N4, H9N5, H9N6, H9N7, H9N8, H9N9. In some embodiments, a cTRP disclosed herein comprising two or more protomers, wherein each protomer is modified with a different subtype of influenza virus.

In certain embodiments, the cargo molecule is a protein.

In certain embodiments, the protein is selected from the group consisting of an immunogen or a cytokine.

In certain embodiments, the cargo molecule is a VHH binder, a human TSLPR ectodomain, or PD-L1, a cytokine for T cell expansion, a Notch ligand an immunogenic peptide, a peptide adjuvant, a single-chain class I MHC domain, a peptide binding domain, phosphotyrosine binding domain, LIM domain, SAM domain, PDZ domain, FERM domain, Pleckstrin homology domain, VVVV domain, oligosaccharide binding (OB) domain, Immunoglobulin domain (IgG), single chain MHC protein, tumor specific antigen (such as CD19 or ROR1), receptor ectodomain, isolated antigenic or adjuvant peptide, small molecule ligand binding domain (e.g., maltose binding protein) and enzyme domain (for example, trypsin), SH2, Protein L, cytokines (e.g., IL-2; IL-17c, IL-3); Notch ligands (e.g., Delta or Jagged); immunogenic peptides; peptide adjuvants; single chain variable fragment nanobodies (Nbs); viral, prokaryotic or eukaryotic immunogen; or an individual member of a catalytic pathway.

In certain embodiments, the cargo molecule is an immunogen.

In certain embodiments, the immunogen is TSLPR or CD3.

In certain embodiments, the cargo molecule is a B cell receptor ligand.

In certain embodiments, the cargo molecule is a TSLPR Immunogen.

In certain embodiments, the TSLPR immunogen is MDT-001741 (Canine_TSLPR-aSym1-His (SEQ ID NO:23)); MDT-001742 (Murine_TSLPR-aSym2 (SEQ ID NO:24)); MDT-001743 (Rat_TSLPR-aSym2 (SEQ ID NO:25)); MDT-001744 (Cyno_TSLPR-aSym3-sFLAG (SEQ ID NO:26)) or MDT-001745 (Human_TSLPR-aSym4-StrepII (SEQ ID NO:27)). In some embodiments, a cTRP comprises protomers of MDT-001741 (Canine_TSLPR-aSym1-His (SEQ ID NO: 23)), MDT-001742 (Murine_TSLPR-aSym2 (SEQ ID NO:24)), MDT-001744 (Cyno_TSLPR-aSym3-sFLAG (SEQ ID NO:26)), and MDT-001745 (Human_TSLPR-aSym4-StrepII (SEQ ID NO:27)) at molar/number ratio of 1:1:1:1. In some embodiments, a cTRP comprises protomers of MDT-001741 (Canine_TSLPR-aSym1-His (SEQ ID NO:23)), MDT-001743 (Rat_TSLPR-aSym2 (SEQ ID NO:25)), MDT-001744 (Cyno_TSLPR-aSym3-sFLAG (SEQ ID NO:26)), and MDT-001745 (Human_TSLPR-aSym4-StrepII (SEQ ID NO:27)) at molar/number ratio of 1:1:1:1.

In certain embodiments, the cargo molecule is a PDL1 ectodomain.

In certain embodiments, the cargo molecule is human, rat, murine cynomolgus or canine PDL1 ectodomain.

In certain embodiments, the cargo molecule is a T-cell expansion reagent.

In certain embodiments, the cargo molecule is a CD3 engaging domain.

In certain embodiments, the cargo molecule is a co-stimulatory domain.

In certain embodiments, the cargo molecule is CD28, CD27, 4-1BB, OX40, or ICOS.

In certain embodiments, the cargo molecule is a ligand of CAR.

In certain embodiments, the cargo molecule is B7H3 ecto, Her2 ecto, EGFR ecto, or IL17Ra ecto.

In certain embodiments, the cargo molecule is Influenza HA.

In certain embodiments, the cargo molecule is a nucleic acid.

In certain embodiments, the cargo molecule is a short hairpin RNA (shRNA), an siRNA, an antisense oligonucleotide, or a component of CRISPR/CAS9 or other gene editing molecules.

In certain embodiments, the cargo molecule is a substrate channeling molecule.

In certain embodiments, the cargo molecule is a substrate channeling enzyme.

In certain embodiments, the cargo molecule is covalently conjugated using sortase.

In certain embodiments, the protomer comprises a recognition motif LPXTG (SEQ ID NO: 44) wherein X can be substituted with any amino acid at the c-terminus; and/or a protomer comprises pentaglycine, triglycine, or diglycine motif on the N-terminus. The recognition motif on the functional domain can be either LPXTG (SEQ ID NO:44) at the c-terminus or pentaglycine, triglycine, or diglycine motif on the N-terminus.

In certain embodiments, the cargo molecule comprises a recognition motif LPXTG (SEQ ID NO: 44) wherein X can be substituted with any amino acid at the c-terminus, and comprises pentaglycine, triglycine, or diglycine motif on the N-terminus.

Multi-species immunogens: In certain embodiments, the cargo molecule of a given protomer is a homologous protein immunogen from a different species. In certain embodiments, the cargo molecule of a given protomer is a homologous protein immunogen from a different species selected to specifically activate B cells that recognize all presented immunogens.

Multi-strain influenza: In certain embodiments, the cargo molecule of a given protomer is an influenza viral glycoprotein immunogen from different strains. In certain embodiments, the cargo molecule of a given protomer is an influenza viral glycoprotein immunogen from different strains to specifically activate B cells that recognize all presented strains.

Multi-antigen presentation for specific CAR T cell expansion: In certain embodiments, the cargo molecule of a given protomer is an antigen (or its ectodomain) recognized by chimeric antigen receptor. In certain embodiments, the cargo molecule of a given protomer is an antigen recognized by chimeric antigen receptor to specifically activate CAR T cells that recognize all presented antigens.

Multi-protein presentation for T cell expansion: In certain embodiments, the cargo molecule of a given protomer is a T cell engaging protein that synergistically activates T cells with co-presented cargo.

### Production of Asymmetrical Circular Tandem Repeat Proteins

Protein Expression and Purification from Human 293 Cells.

The ‘Daedalus’ human cell line expression platform was employed for the production and purification of secreted cTRP proteins, using methods described previously17. The expression system makes use of suspension-adapted HEK293 Freestyle cells and a highly optimized lentiviral transduction protocol to generate cell lines that secrete proteins at high levels. The lentiviral vector contains a cis-linked fluorescent protein reporter driven by an internal ribosome entry site (IRES) that allows for tracking of relative protein expression levels. In certain embodiments, mammalian proteins were purified directly from conditioned media using HisTrap FF Crude columns (GE #17528601) and subsequently polished on a Superose 6 10/300 GL SEC column (GE #17517201) using an AKTA pure 25 instrument.

### Negative Stain Electron Microscopy Analysis (EM).

Negative stained specimens for Transmission Electron Microscopy were prepared using methods previously described21. Briefly, 4 μL of a cTRP solution at ~40 to 50 nM was applied to the surface of a freshly glow-discharged carbon film coated Copper grid held at the tip of an anti-capillary tweezer. The solution was adsorbed for 15-60 seconds. The grid was then washed 3 times by touching the surface to a 20 μL water droplet on the surface of a parafilm strip. Each time, the water attached to the grid was removed by briefly touching the surface of a nearby filter paper. The washing process was repeated twice with 20 μL and 40 μL of 0.7% Uranyl Format (UF). The last droplet of UF was allowed to remain in contact with the grid for 15 to 60 seconds prior to removing the filter paper. The stained grid was air-dried for 5 minutes prior to storage in a grid box. Grids were analyzed by TEM using a JEOL1400 microscope operating at 120 kV. The images were recorded using a GATAN CCD detector at a nominal magnification of 60,000× at the surface of the fluorescent screen.

### Synthesis and Production of Protomers Containing Non-Canonical Amino Acids

Mutually orthogonal chemical handles can be converted into non-canonical amino acids through chemical functionalization. As a less-involved example, in Hoffmann J E, Dziuba D, Stein F, Schultz C. A Bifunctional Noncanonical Amino Acid: Synthesis, Expression, and Residue-Specific Proteome-wide Incorporation. Biochemistry. 2018; 57(31):4747-4752, the authors perform a DIEA amidation reaction between vendor-sourced Fmoc-Lys-OH and a chemical handle. More complex methods may also be used. In Roth S, Drewe W C, Thomas N R. A concise and scalable route to L-azidohomoalanine. Nat Protoc. 2010; 5(12):1967-1973, the authors directly conjugate an azide onto an amino acid precursor, then chemically process the precursor into L-azidohomoalanine. For example, starting from N-Boc-O-Bn-1-aspartic acid, gram quantities of L-azidohomoalanine (L-Aha) hydrochloride can be prepared with high purity, and the products can be incorporated into proteins using L-methionine auxotrophs.

Non-canonical amino acids can be genetically inserted into asymmetric cTRPs either through amino acid depletion/substitution, or the use of mutant tRNA synthetase and orthogonal tRNAs. As early as 2002, Kiick K L, Saxon E, Tirrell D A, Bertozzi C R. Incorporation of azides into recombinant proteins for chemoselective modification by the Staudinger ligation. P Natl Acad Sci USA. 2002; 99(1):19-24, utilized Methionine auxotrophic E. coli and substituted various Met-analog ncAA into their growth media to incorporate their chemical handle globally at Met sites. More modern methods utilize genetic code expansion to site-specifically add ncAA into a translating protein within mammalian cells. Many common methods used today are based on the the PylRS and tRNApyl Amber (TAG) stop codon suppression adopted from Bacteria and Archea. See Saleh A M, Wilding K M, Calve S, Bundy B C, Kinzer-Ursem T L. Non-canonical amino acid labeling in proteomics and biotechnology. J Biol Eng. 2019; 13:43.

Uses of cTRPs

As discussed herein, there are many possible uses of the disclosed cTRPs for synthetic biology and bioengineering. Exemplary applications for the cTRPs include the incorporation and display of multiple copies of protein or peptide ligands for use in cell-stimulating growth factors and in vaccine development. Without being bound by theory, it is hypothesized that the display complex arrangements of multiple peptide and/or protein moieties onto stable protein nanoparticles, with well-defined symmetry and distances separating those individual components, will facilitate the creation of growth factor reagents and vaccines with exceptional biological activity as a result of (i) formation of high avidity interactions at the cell surface, and (ii) the potential to induce clustering of receptor complexes when the nanoparticle encounters its extracellular targets in vivo.

Cell manufacturing (e.g., the expansion of hematopoietic stem cells (and especially from limiting starting pools of such cells such as infant cord blood)) involves the addition of mixtures of cytokines and growth factors in various combinations to cell cultures. The overall biological activities of commercial cytokine and growth factor preparations in cell culture are suboptimal, being limited by the hostile environment of the cell incubator and media (which results in rapid protein degradation) and low activity in the wide-open spaces and volumes of the incubator (which negates the close intercellular distances that govern cytokine action in a living body). Therefore, the consumption, cost and effectiveness of these reagents is a clear target for improvement by a next generation of improved cell culture reagents, such as the cTRPs described herein. When used for cell manufacturing, the extreme physical and thermal stability of the underlying cTRP protein scaffolds (which have been shown to remain intact at temperatures up to 95° C.) greatly increases the stability and lifetime of the molecules used during manufacturing.

### Antibodies and Antibody Fragments

As used herein, the term “antibody” includes a single-chain variable fragment (scFv or “nanobody”), humanized, fully human or chimeric antibodies, single-chain antibodies, diabodies, and antigen-binding fragments of antibodies that do not contain the Fc region (e.g., Fab fragments). In certain embodiments, the antibody is a human antibody or a humanized antibody. A “humanized” antibody contains only the three CDRs (complementarity determining regions) and sometimes a few carefully selected “framework” residues (the non-CDR portions of the variable regions) from each donor antibody variable region recombinantly linked onto the corresponding frameworks and constant regions of a human antibody sequence. A “fully humanized antibody” is created in a hybridoma from mice genetically engineered to have only human-derived antibody genes or by selection from a phage-display library of human-derived antibody genes.

A scFv is a fusion protein of the variable region of the heavy (VH) and light chains (VL) of an immunoglobulin that is connected by means of a linker peptide. The linker is usually short, about 10-25 amino acids in length. If flexibility is important, the linker will contain a significant number of glycines. If solubility is important, serines or theonines will be utilized in the linker. The linker may link the amino-terminus of the VH to the carboxy-terminus of the VL, or the linker may link the carboxy-terminus of the VH to the amino-terminus of the VL. Divalent (also called bivalent) scFvs can be generated by linking two scFvs. For example, a divalent scFv can be made by generating a single peptide containing two VH and two VL regions. Alternatively, two peptides, each containing a single VH and a single VL region can be dimerized (also called “diabodies”). Holliger et al., “Diabodies: small bivalent and bispecific antibody fragments,” PNAS, July 1993, 90:6444-6448. Bivalency allows antibodies to bind to multimeric antigens with high avidity, and bispecificity allows the cross-linking of two antigens.

As used herein, the term “monoclonal antibody” refers to an antibody obtained from a group of substantially homogeneous antibodies, that is, an antibody group wherein the antibodies constituting the group are homogeneous except for naturally occurring mutants that exist in a small amount. Monoclonal antibodies are highly specific and interact with a single antigenic site. Furthermore, each monoclonal antibody targets a single antigenic determinant (epitope) on an antigen, as compared to common polyclonal antibody preparations that typically contain various antibodies against diverse antigenic determinants. In addition to their specificity, monoclonal antibodies are advantageous in that they are typically produced from hybridoma cultures not contaminated with other immunoglobulins.

The adjective “monoclonal” indicates a characteristic of antibodies obtained from a substantially homogeneous group of antibodies, and does not specify antibodies produced by a particular method. For example, a monoclonal antibody to be used in the present invention can be produced by, for example, hybridoma methods (Kohler and Milstein, Nature 256:495, 1975) or recombination methods (U.S. Pat. No. 4,816,567). The monoclonal antibodies used in the present invention can be also isolated from a phage antibody library (Clackson et al., Nature 352:624-628, 1991; Marks et al., J. Mol. Biol. 222:581-597, 1991). The monoclonal antibodies of the present invention may comprise “chimeric” antibodies (immunoglobulins), wherein a part of a heavy (H) chain and/or light (L) chain is derived from a specific species or a specific antibody class or subclass, and the remaining portion of the chain is derived from another species, or another antibody class or subclass. Furthermore, mutant antibodies and antibody fragments thereof are also comprised in the present invention (U.S. Pat. No. 4,816,567; Morrison et al., Proc. Natl. Acad. Sci. USA 81:6851-6855, 1984).

As used herein, the term “mutant antibody” refers to an antibody comprising a variant amino acid sequence in which one or more amino acid residues have been altered. For example, the variable region of an antibody can be modified to improve its biological properties, such as antigen binding. Such modifications can be achieved by site-directed mutagenesis (see Kunkel, Proc. Natl. Acad. Sci. USA 82:488 (1985)), PCR-based mutagenesis, cassette mutagenesis, and the like. Such mutants comprise an amino acid sequence which is at least 70% identical to the amino acid sequence of a heavy or light chain variable region of the antibody, more specifically at least 75%, even more specifically at least 80%, still more specifically at least 85%, yet more specifically at least 90%, and most specifically at least 95% identical. As used herein, the term “sequence identity” is defined as the percentage of residues identical to those in the antibody's original amino acid sequence, determined after the sequences are aligned and gaps are appropriately introduced to maximize the sequence identity as necessary.

Specifically, the identity of one nucleotide sequence or amino acid sequence to another can be determined using the algorithm BLAST, by Karlin and Altschul (Proc. Natl. Acad. Sci. USA, 90:5873-5877, 1993). Programs such as BLASTN and BLASTX were developed based on this algorithm (Altschul et al., J. Mol. Biol. 215:403-410, 1990). To analyze nucleotide sequences according to BLASTN based on BLAST, the parameters are set, for example, as score=100 and wordlength=12. On the other hand, parameters used for the analysis of amino acid sequences by BLASTX based on BLAST include, for example, score=50 and wordlength=3. Default parameters for each program are used when using the BLAST and Gapped BLAST programs. Specific techniques for such analyses are known in the art (see the website of the National Center for Biotechnology Information (NCBI), Basic Local Alignment Search Tool (BLAST); www.ncbi.nlm.nih.gov).

Polyclonal and monoclonal antibodies can be prepared by methods known to those skilled in the art.

In another embodiment, antibodies or antibody fragments can be isolated from an antibody phage library, produced by using the technique reported by McCafferty et al. (Nature 348:552-554 (1990)). Clackson et al. (Nature 352:624-628 (1991)) and Marks et al. (J. Mol. Biol. 222:581-597 (1991)) reported on the respective isolation of mouse and human antibodies from phage libraries. There are also reports that describe the production of high affinity (nM range) human antibodies based on chain shuffling (Marks et al., Bio/Technology 10:779-783 (1992)), and combinatorial infection and in vivo recombination, which are methods for constructing large-scale phage libraries (Waterhouse et al., Nucleic Acids Res. 21:2265-2266 (1993)). These technologies can also be used to isolate monoclonal antibodies, instead of using conventional hybridoma technology for monoclonal antibody production.

In certain embodiments, an amino acid residue is mutated into one that allows the properties of the amino acid side-chain to be conserved. Examples of the properties of amino acid side chains comprise: hydrophobic amino acids (A, I, L, M, F, P, W, Y, V), hydrophilic amino acids (R, D, N, C, E, Q, G, H, K, S, T), and amino acids comprising the following side chains: aliphatic side-chains (G, A, V, L, I, P); hydroxyl group-containing side-chains (S, T, Y); sulfur atom-containing side-chains (C, M); carboxylic acid- and amide-containing side-chains (D, N, E, Q); base-containing side-chains (R, K, H); and aromatic-containing side-chains (H, F, Y, W). The letters within parenthesis indicate the one-letter amino acid codes. Amino acid substitutions within each group are called conservative substitutions. It is well known that a polypeptide comprising a modified amino acid sequence in which one or more amino acid residues is deleted, added, and/or substituted can retain the original biological activity (Mark D. F. et al., Proc. Natl. Acad. Sci. U.S.A. 81:5662-5666 (1984); Zoller M. J. and Smith M., Nucleic Acids Res. 10:6487-6500 (1982); Wang A. et al., Science 224:1431-1433; Dalbadie-McFarland G. et al., Proc. Natl. Acad. Sci. U.S.A. 79:6409-6413 (1982)). The number of mutated amino acids is not limited, but in general, the number falls within 40% of amino acids of each CDR, and specifically within 35%, and still more specifically within 30% (e.g., within 25%). The identity of amino acid sequences can be determined as described herein.

Recombinant antibodies can be artificially modified to reduce heterologous antigenicity against humans. Examples include chimeric antibodies and humanized antibodies. These modified antibodies can be produced using known methods. A chimeric antibody includes an antibody comprising variable and constant regions of species that are different to each other, for example, an antibody comprising the antibody heavy chain and light chain variable regions of a nonhuman mammal such as a mouse, and the antibody heavy chain and light chain constant regions of a human. Such an antibody can be obtained by (1) ligating a DNA encoding a variable region of a mouse antibody to a DNA encoding a constant region of a human antibody; (2) incorporating this into an expression vector; and (3) introducing the vector into a host for production of the antibody.

A humanized antibody, which is also called a reshaped human antibody, may be obtained by substituting an H or L chain complementarity determining region (CDR) of an antibody of a nonhuman mammal such as a mouse, with the CDR of a human antibody. Conventional genetic recombination techniques for the preparation of such antibodies are known (see, for example, Jones et al., Nature 321:522-525 (1986); Reichmann et al., Nature 332:323-329 (1988); Presta Curr. Op. Struct. Biol. 2:593-596 (1992)). Specifically, a DNA sequence designed to ligate a CDR of a mouse antibody with the framework regions (FRs) of a human antibody is synthesized by PCR, using several oligonucleotides constructed to comprise overlapping portions at their ends. A humanized antibody can be obtained by (1) ligating the resulting DNA to a DNA that encodes a human antibody constant region; (2) incorporating this into an expression vector; and (3) transfecting the vector into a host to produce the antibody (see, European Patent Application No. EP 239,400, and International Patent Application No. WO 96/02576). Human antibody FRs that are ligated via the CDR are selected where the CDR forms a favorable antigen-binding site. The humanized antibody may comprise additional amino acid residue(s) that are not included in the CDRs introduced into the recipient antibody, nor in the framework sequences. Such amino acid residues are usually introduced to more accurately optimize the antibody's ability to recognize and bind to an antigen. For example, as necessary, amino acids in the framework region of an antibody variable region may be substituted such that the CDR of a reshaped human antibody forms an appropriate antigen-binding site (Sato, K. et al., Cancer Res. (1993) 53, 851-856).

The isotypes of the antibodies of the present invention are not limited. The isotypes include, for example, IgG (IgG1, IgG2, IgG3, and IgG4), IgM, IgA (IgAQ1 and IgA2), IgD, and IgE. The antibodies of the present invention may also be antibody fragments comprising a portion responsible for antigen binding, or a modified fragment thereof. The term “antibody fragment” refers to a portion of a full-length antibody, and generally to a fragment comprising an antigen-binding domain or a variable region. Such antibody fragments include, for example, Fab, F(ab′) 2, Fv, single-chain Fv (scFv) which comprises a heavy chain Fv and a light chain Fv coupled together with an appropriate linker, diabody (diabodies), linear antibodies, and multispecific antibodies prepared from antibody fragments. Previously, antibody fragments were produced by digesting natural antibodies with a protease; currently, methods for expressing them as recombinant antibodies using genetic engineering techniques are also known (see Morimoto et al., Journal of Biochemical and Biophysical Methods 24:107-117 (1992); Brennan et al., Science 229:81 (1985); Co, M. S. et al., J. Immunol., 1994, 152, 2968-2976; Better, M. & Horwitz, A. H., Methods in Enzymology, 1989, 178, 476-496, Academic Press, Inc.; Plueckthun, A. & Skerra, A., Methods in Enzymology, 1989, 178, 476-496, Academic Press, Inc.; Lamoyi, E., Methods in Enzymology, 1989, 121, 663-669; Bird, R. E. et al., TIBTECH, 1991, 9, 132-137).

An “Fv” fragment is the smallest antibody fragment and contains a complete antigen recognition site and a binding site. This region is a dimer (VH-VL dimer) wherein the variable regions of each of the heavy chain and light chain are strongly connected by a noncovalent bond. The three CDRs of each of the variable regions interact with each other to form an antigen-binding site on the surface of the VH-VL dimer. In other words, a total of six CDRs from the heavy and light chains function together as an antibody's antigen-binding site. However, a variable region (or a half Fv, which contains only three antigen-specific CDRS) alone is also known to be able to recognize and bind to an antigen, although its affinity is lower than the affinity of the entire binding site. Thus, a specific antibody fragment of the present invention is an Fv fragment but is not limited thereto. Such an antibody fragment may be a polypeptide which comprises an antibody fragment of heavy or light chain CDRs which are conserved, and which can recognize and bind its antigen.

A Fab fragment (also referred to as F(ab)) also contains a light chain constant region and heavy chain constant region (CH1). For example, papain digestion of an antibody produces the two kinds of fragments: an antigen-binding fragment, called a Fab fragment, containing the variable regions of a heavy chain and light chain, which serve as a single antigen-binding domain; and the remaining portion, which is called an “Fc” because it is readily crystallized. A Fab′ fragment is different from a Fab fragment in that a Fab′ fragment also has several residues derived from the carboxyl terminus of a heavy chain CH1 region, which contains one or more cysteine residues from the hinge region of an antibody. A Fab′ fragment is, however, structurally equivalent to Fab in that both are antigen-binding fragments which comprise the variable regions of a heavy chain and light chain, which serve as a single antigen-binding domain. Herein, an antigen-binding fragment comprising the variable regions of a heavy chain and light chain which serve as a single antigen-binding domain, and which is equivalent to that obtained by papain digestion, is referred to as a “Fab-like antibody,” even when it is not identical to an antibody fragment produced by protease digestion. Fab′-SH is Fab′ with one or more cysteine residues having free thiol groups in its constant region. A F(ab′) fragment is produced by cleaving the disulfide bond between the cysteine residues in the hinge region of F(ab′) 2. Other chemically crosslinked antibody fragments are also known to those skilled in the art. Pepsin digestion of an antibody yields two fragments; one is a F(ab′) 2 fragment which comprises two antigen-binding domains and can cross-react with antigens, and the other is the remaining fragment (referred to as pFc′). Herein, an antibody fragment equivalent to that obtained by pepsin digestion is referred to as a “F(ab′) 2-like antibody” when it comprises two antigen-binding domains and can cross-react with antigens. Such antibody fragments can also be produced, for example, by genetic engineering. Such antibody fragments can also be isolated, for example, from the antibody phage library described above. Alternatively, F(ab′) 2-SH fragments can be recovered directly from hosts, such as E. coli, and then allowed to form F(ab′) 2 fragments by chemical crosslinking (Carter et al., Bio/Technology 10:163-167 (1992)). In an alternative method, F(ab′) 2 fragments can be isolated directly from a culture of recombinant hosts.

The term “diabody (Db)” refers to a bivalent antibody fragment constructed by gene fusion (for example, P. Holliger et al., Proc. Natl. Acad. Sci. USA 90:6444-6448 (1993), EP 404,097, WO 93/11161). In general, a diabody is a dimer of two polypeptide chains. In the each of the polypeptide chains, a light chain variable region (VL) and a heavy chain variable region (VH) in an identical chain are connected via a short linker, for example, a linker of about five residues, so that they cannot bind together. Because the linker between the two is too short, the VL and VH in the same polypeptide chain cannot form a single chain V region fragment, but instead form a dimer. Thus, a diabody has two antigen-binding domains. When the VL and VH regions against the two types of antigens (a and b) are combined to form VLa-VHb and VLb-VHa via a linker of about five residues, and then co-expressed, they are secreted as bispecific Dbs. The antibodies of the present invention may be such Dbs.

A single-chain antibody (also referred to as “scFv”) can be prepared by linking a heavy chain V region and a light chain V region of an antibody (for a review of scFv see Pluckthun “The Pharmacology of Monoclonal Antibodies” Vol. 113, eds. Rosenburg and Moore, Springer Verlag, N.Y., pp. 269-315 (1994)). Methods for preparing single-chain antibodies are known in the art (see, for example, U.S. Pat. Nos. 4,946,778; 5,260,203; 5,091,513; and 5,455,030). In such scFvs, the heavy chain V region and the light chain V region are linked together via a linker, such as a polypeptide linker (Huston, J. S. et al., Proc. Natl. Acad. Sci. U.S.A, 1988, 85, 5879-5883). The heavy chain V region and the light chain V region in a scFv may be derived from the same antibody, or from different antibodies. The peptide linker used to ligate the V regions may be any single-chain peptide consisting of 12 to 19 residues. A DNA encoding a scFv can be amplified by PCR using, as a template, either the entire DNA, or a partial DNA encoding a desired amino acid sequence, selected from a DNA encoding the heavy chain or the V region of the heavy chain of the above antibody, and a DNA encoding the light chain or the V region of the light chain of the above antibody; and using a primer pair that defines the two ends. Further amplification can be subsequently conducted using a combination of the DNA encoding the peptide linker portion, and the primer pair that defines both ends of the DNA to be ligated to the heavy and light chain respectively. After constructing DNAs encoding scFvs, conventional methods can be used to obtain expression vectors comprising these DNAs, and hosts transformed by these expression vectors. Furthermore, scFvs can be obtained according to conventional methods using the resulting hosts. These antibody fragments can be produced in hosts by obtaining genes that encode the antibody fragments and expressing these as outlined above. Antibodies bound to various types of molecules, such as polyethylene glycols (PEGs), may be used as modified antibodies. Methods for modifying antibodies are already established in the art. The term “antibody” in the present invention also encompasses the above-described antibodies.

The antibodies obtained can be purified to homogeneity. The antibodies can be isolated and purified by a method routinely used to isolate and purify proteins. The antibodies can be isolated and purified by the combined use of one or more methods appropriately selected from column chromatography, filtration, ultrafiltration, salting out, dialysis, preparative polyacrylamide gel electrophoresis, and isoelectro-focusing, for example (Strategies for Protein Purification and Characterization: A Laboratory Course Manual, Daniel R. Marshak et al. eds., Cold Spring Harbor Laboratory Press (1996); Antibodies: A Laboratory Manual. Ed Harlow and David Lane, Cold Spring Harbor Laboratory, 1988). Such methods are not limited to those listed above. Chromatographic methods include affinity chromatography, ion exchange chromatography, hydrophobic chromatography, gel filtration, reverse-phase chromatography, and adsorption chromatography. These chromatographic methods can be practiced using liquid phase chromatography, such as HPLC and FPLC. Columns to be used in affinity chromatography include protein A columns and protein G columns. For example, protein A columns include Hyper D, POROS, and Sepharose F. F. (Pharmacia). Antibodies can also be purified by utilizing antigen binding, using carriers on which antigens have been immobilized.

The protomers and the cTRP of the present invention can be formulated with pharmaceutically acceptable carriers and/or additives. The present invention relates to compositions (including reagents and pharmaceuticals) comprising the protomers and/or the cTRP of the invention, and pharmaceutically acceptable carriers and/or additives. Exemplary carriers include surfactants (for example, PEG and Tween), excipients, antioxidants (for example, ascorbic acid), coloring agents, flavoring agents, preservatives, stabilizers, buffering agents (for example, phosphoric acid, citric acid, and other organic acids), chelating agents (for example, EDTA), suspending agents, isotonizing agents, binders, disintegrators, lubricants, fluidity promoters, and corrigents. However, the carriers that may be employed in the present invention are not limited to this list. In fact, other commonly used carriers can be appropriately employed: light anhydrous silicic acid, lactose, crystalline cellulose, mannitol, starch, carmelose calcium, carmelose sodium, hydroxypropylcellulose, hydroxypropylmethyl cellulose, polyvinylacetaldiethylaminoacetate, polyvinylpyrrolidone, gelatin, medium chain fatty acid triglyceride, polyoxyethylene hydrogenated castor oil 60, sucrose, carboxymethylcellulose, corn starch, inorganic salt, and so on. The composition may also comprise other low-molecular-weight polypeptides, proteins such as serum albumin, gelatin, and immunoglobulin, and amino acids such as glycine, glutamine, asparagine, arginine, and lysine. When the composition is prepared as an aqueous solution for injection, it can comprise an isotonic solution comprising, for example, physiological saline, dextrose, and other adjuvants, including, for example, D-sorbitol, D-mannose, D-mannitol, and sodium chloride, which can also contain an appropriate solubilizing agent, for example, alcohol (for example, ethanol), polyalcohol (for example, propylene glycol and PEG), and non-ionic detergent (polysorbate 80 and HCO-50).

### Vaccines and Therapeutic Agents

In certain embodiments, the present invention provides vaccines for use to protect mammals against or to treat an infection.

In certain embodiments, the present invention provides therapeutic agents that inhibit an infection.

As used herein, the term “therapeutic agent” or “therapeutic complex” refers to any agent or material that has a beneficial effect on the subject recipient. Thus, “therapeutic agent” embraces both therapeutic and prophylactic molecules having nucleic acid or protein components.

To prepare a vaccine, the cTRP is prepared. The cTRP may then be adjusted to an appropriate concentration, optionally combined with a suitable vaccine adjuvant, and packaged for use.

In certain embodiments, the cTRP is adjusted to an appropriate concentration and can be formulated with any suitable adjuvant, diluent, pharmaceutically acceptable carrier, or any combination thereof. As used herein the phrase “pharmaceutically acceptable carrier” is intended to include any and all solvents, dispersion media, coatings, antibacterial and antifungal agents, isotonic and absorption delaying agents, excipients and the like, compatible with pharmaceutical administration. The use of such media and agents for pharmaceutically active substances is well known in the art. Physiologically acceptable vehicles may be used as carriers and/or diluents. A pharmaceutically acceptable vehicle is understood to designate a compound or a combination of compounds entering into a pharmaceutical or immunogenic composition which does not cause side effects, and which makes it possible, for example, to facilitate the administration of the active compound, to increase its life and/or its efficacy in the body, to increase its solubility in solution or alternatively to enhance its preservation. These pharmaceutically acceptable vehicles are well known and will be adapted by persons skilled in the art according to the nature and the mode of administration of the active compound chosen. These include, but are not limited to, water, Ringer's solution, an appropriate isotonic medium, glycerol, ethanol and other conventional solvents, phosphate buffered saline, and the like.

“Treating” as used herein refers to ameliorating at least one symptom of, curing and/or preventing the development of a given disease or condition.

“Antigen” refers to a molecule capable of being bound by an antibody. An antigen is additionally capable of being recognized by the immune system and/or being capable of inducing a humoral immune response and/or cellular immune response leading to the activation of B- and/or T-lymphocytes. An antigen can have one or more epitopes (B- and/or T-cell epitopes). Antigens as used herein may also be mixtures of several individual antigens. “Antigenic determinant” refers to that portion of an antigen that is specifically recognized by either B- or T-lymphocytes. B-lymphocytes responding to antigenic determinants produce antibodies, whereas T-lymphocytes respond to antigenic determinants by proliferation and establishment of effector functions critical for the mediation of cellular and/or humoral immunity.

An “immune response” refers to a humoral immune response and/or cellular immune response leading to the activation or proliferation of B- and/or T-lymphocytes and/or and antigen presenting cells. In some instances, however, the immune responses may be of low intensity and become detectable only when using at least one substance in accordance with the invention. “Immunogenic” refers to an agent used to stimulate the immune system of a living organism, so that one or more functions of the immune system are increased and directed towards the immunogenic agent. An “immunogenic polypeptide” is a polypeptide that elicits a cellular and/or humoral immune response, whether alone or linked to a carrier. Preferably, antigen presenting cell may be activated.

A substance that “enhances” an immune response refers to a substance in which an immune response is observed that is greater or intensified or deviated in any way with the addition of the substance when compared to the same immune response measured without the addition of the substance. For example, the lytic activity of cytotoxic T lymphocytes (CTLs) can be measured, e.g. using a 51Cr release assay, in samples obtained with and without the use of the substance during immunization. The amount of the substance at which the CTL lytic activity is enhanced as compared to the CTL lytic activity without the substance is said to be an amount sufficient to enhance the immune response of the animal to the antigen. In certain embodiments, the immune response in enhanced by a factor of at least about 2, such as by a factor of about 3 or more. The amount or type of cytokines secreted may also be altered. Alternatively, the amount of antibodies induced or their subclasses may be altered.

The terms “immunize,” “immunization” or related terms refer to conferring the ability to mount a substantial immune response (comprising antibodies and/or cellular immunity such as effector CTL) against a target antigen or epitope. These terms do not require that complete immunity be created, but rather that an immune response be produced which is substantially greater than baseline. For example, a mammal may be considered to be immunized against a target antigen if the cellular and/or humoral immune response to the target antigen occurs following the application of methods of the invention.

The term “immunotherapeutic” refers to a composition for the treatment of diseases, disorders or conditions. More specifically, the term is used to refer to a method of treatment wherein a beneficial immune response is generated by vaccination or by transfer of immune molecules. An “immunologically effective amount” refers to an amount of a composition sufficient to induce an immune response in an individual when introduced into that individual. In the context of active immunization, the term is synonymous with “immunogenically effective amount.” The amount of a composition necessary to be immunologically effective varies according many factors including to the composition, the presence of other components in the composition, the antigen, the route of immunization, the individual, the prior immune or physiologic state etc.

The term “epitope” refers to basic element or smallest unit of recognition by an individual antibody or T-cell receptor, and thus the particular domain, region or molecular structure to which said antibody or T-cell receptor binds. An antigen may consist of numerous epitopes while a hapten, typically, may possess few epitopes. As used herein “correspond essentially to” refers to an epitope that will elicit an immunological response at least substantially equivalent to the response generated by the native epitope. An immunological response to a composition or vaccine is the development in the host of a cellular and/or antibody-mediated immune response to the polypeptide or vaccine of interest. Usually, such a response consists of the subject producing antibodies, B cell, helper T cells, suppressor T cells, and/or cytotoxic T cells directed specifically to an antigen or antigens included in the composition or vaccine of interest.

Vaccines of the present invention can also include effective amounts of immunological adjuvants, known to enhance an immune response. An “effective amount” refers to an amount necessary or sufficient to realize a desired biologic effect. An effective amount of the composition would be the amount that achieves this selected result, and such an amount could be determined as a matter of routine by a person skilled in the art. For example, an effective amount for treating an immune system deficiency could be that amount necessary to cause activation of the immune system, resulting in the development of an antigen specific immune response upon exposure to antigen. The term is also synonymous with “sufficient amount.” The effective amount for any particular application can vary depending on such factors as the disease or condition being treated, the particular composition being administered, the size of the subject, and/or the severity of the disease or condition. One of ordinary skill in the art can empirically determine the effective amount of a particular composition of the present invention without necessitating undue experimentation.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1. Production of Asymmetrical Circular Tandem Repeat Proteins

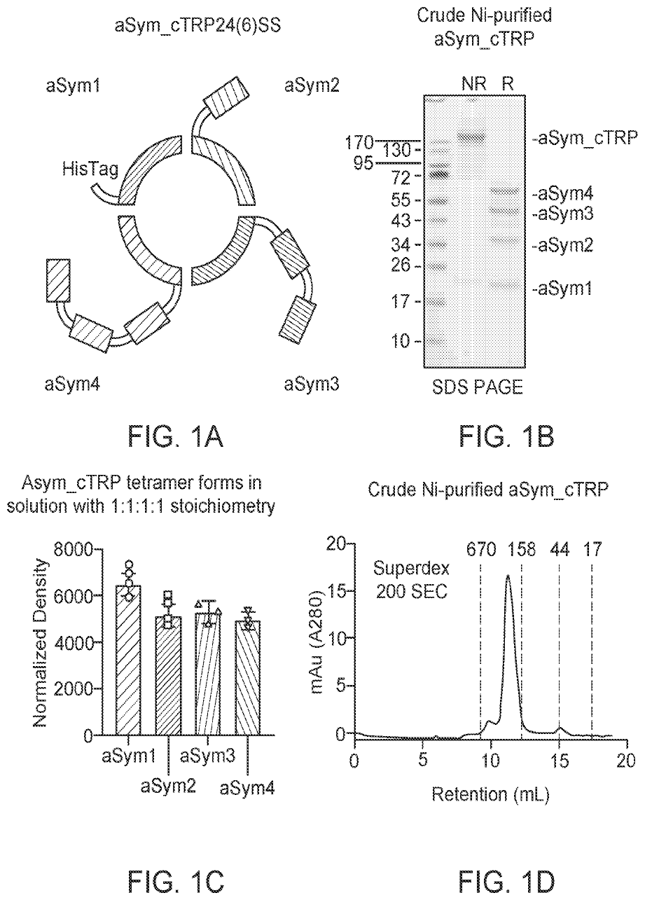

cTRPs are coordinated through weak interchain interactions that are fixed in place by strong disulfide bonds. Leveraging this fact, it was reasoned that placement of the disulfides could be sufficient for asymmetric assembly. For example, all interface residue pairs at a distance of less than 7.0 Å were substituted for cysteines and refolded for validation. Three of the highest confidence substitutions pairs were selected for initial expression. To demonstrate an asymmetric assembly had formed, cargo of different molecular weight was loaded onto each protomer. Unique affinity purification tags are fused at the C-terminus of the protomers to facilitate purification and detection. FIGS. 1A-1D. It was surprisingly discovered that asymmetric circular TRPs could be generated.

### Example 2. Application of Asymmetrical cTRPs-Immunogens

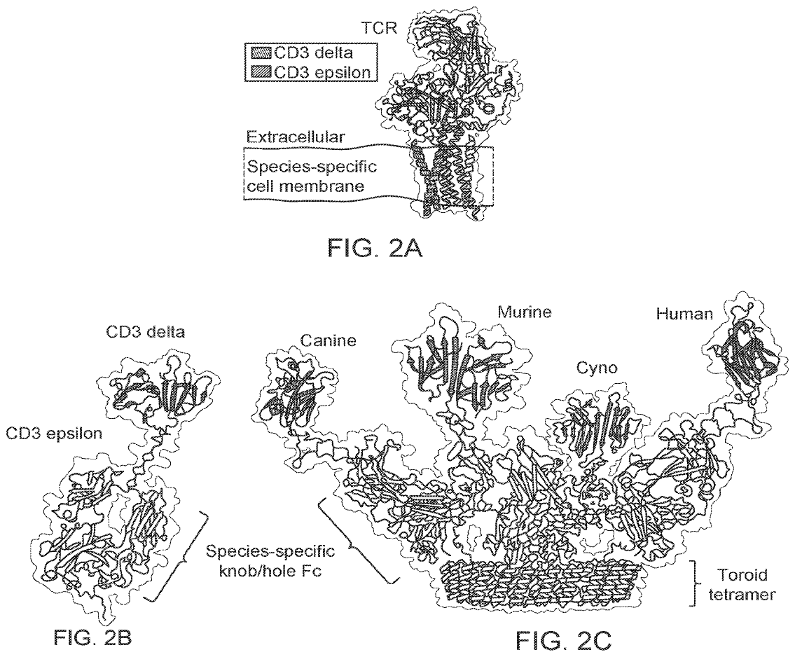

Multi-species cross reactivity is a high value attribute for monoclonal antibodies as it accelerates clinical development by enabling full syngeneic models. There is no current way to bias the antibody response of an immunization towards a conserved epitope to enrich for cross reactivity. Employing the asymmetric cTRP to present the same immunogen from different species could be a way to cluster B cell receptors conditionally to selectively generate such binders. The holoreceptor CD3 would be a high value target given its ability to stimulate T cell cytotoxicity. An immunogen presenting human, murine, cynomolgus, and canine CD3 epsilon/delta heterodimers is assembled in defined positions and at an equal 1:1:1:1 ratio using the asymmetric cTRP scaffold. FIGS. 2A-2C.

### Example 3. Application of Asymmetrical cTRPs-Specific B-Cell Activation

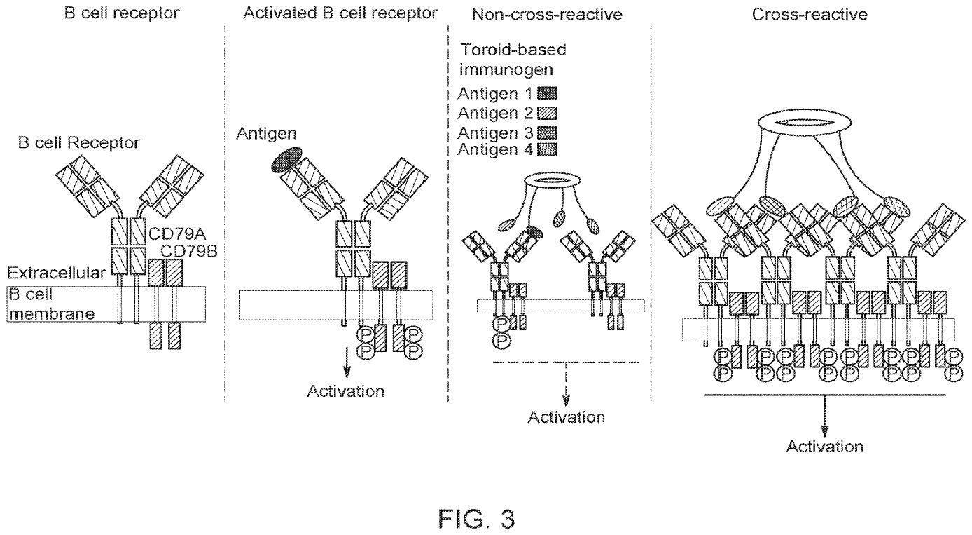

The pathway of antibody production is initiated by interaction of the B cell receptor with a ligand. Engagement of the B cell receptor is transduced to an intracellular signal via the CD79A/B heterodimer. This signal is enhanced by the valency of the ligand which leads to clustering of B cell receptors on the surface of the B cell. Using a scaffold that presents specific immunogens in defined ratios and positions, clustering would only occur when the B cell receptor binds to a conserved epitope shared by all cTRP cargos. For example, using this platform, using conserved epitope of TSLPR (e.g., TSLPR from different mammalian species) as immunogens for conditional activation can enrich for an antibody response of the immunized animal towards cross-reactivity. FIG. 3.

### Example 4. Application of Asymmetrical cTRPs-Multi-Species TSLPR Immunogens

A specific application of asymmetrical cTRPs as multi-species immunogens is demonstrated by fusion of each asymmetric subunit/protomer to a TSLPR antigen each from canine, murine, cynomolgus and human respectively. The ecto-domain of each TSLPR antigen with an engineered or native signal peptide was fused to the asymmetric cTRP protomer at the termini with the use of a flexible linker of arbitrary length. The subunit with the mouse TSLPR antigen was substituted with the analogous antigen from Rat to make an equivalent multi-species immunogen for conducting an immunization campaign in mouse. The sequences of each protomer are found in Table 1.

TABLE 1
Exemplary sequences of each subunit in
multi-species asymmetric cTRP

| Identifier | Sequence |
|---|---|
| MDT-001741 | MRTVLLPWASAAVFLLVTVTVSGDEALEGVLQL |
| (Canine_ | QMINENFEAVQVTWNASQYSGTNLTFLYKLTSD |
| TSLPR- | ETDSQCSNYILQQSRTAGCLLEAKEDEILCFSI |
| aSym1-His) | WNGTRLLLNKCQWISAYLKPSSPKDLNFQWHQE |
| | AITVTCSDLPYKRLLYEIQYKSMEDTEWQSKEG |
| | ETCNVTTDGLDAEKCYFFRARVKTMESSYGPDT |
| | YPSDWSEVIHQQKGELRDSCQEKKLEPKGGGGS |
| | GGGGSCEAIKAAAELGKAGISSEEILELLRAAH |
| | ELGLDPEAIKAAAELGKAGISSEEILELLRAAH |
| | ELGLDPEAIKAAAELGKAGISSEEILELLRAAH |
| | ELGLDPEAIKAAAELGKAGISSEEILELLRAAH |
| | ELGLDPEAIKAAAELGKAGISSEEILELLRAAH |
| | ELGLDPEAIKAAAELGKCGISSEEILELLRAAH |
| | ELGLGGGSHHHHHH |
| | (SEQ ID NO: 23) |
| MDT-001742 | MAWALAVILLPRLLAAAAAAAAVTSRGDVTVVC |
| (Murine_ | HDLETVEVTWGSGPDHHGANLSLEFRYGTGALQ |
| TSLPR- | PCPRYFLSGAGVTSGCILPAARAGLLELALRDG |
| aSym2) | GGAMVFKARQRASAWLKPRPPWNVILLWTPDGD |
| | VTVSWPAHSYLGLDYEVQHRESNDDEDAWQTTS |
| | GPCCDLTVGGLDPARCYDFRVRASPRAAHYGLE |
| | AQPSEWTAVTRLSGAASAASCTASPAPSPALAP |
| | PGGGGSGGGGSPEAIKAAAELGCAGISSEEILE |
| | LLRAAHELGLDPEAIKAAAELGKAGISSEEILE |
| | LLRAAHELGLDPEAIKAAAELGKAGISSEEILE |
| | LLRAAHELGLDPEAIKAAAELGKAGISSEEILE |
| | LLRAAHELGLDPEAIKAAAELGKAGISSEEILE |
| | LLRAAHELGLDPEAIKAAACLGKAGISSEEILE |
| | LLRAAHELGLGGGS |
| | (SEQ ID NO: 24) |
| MDT-001743 | MRAVTWAIVAMLLPRVLGAIPTRTPRTGGVGDT |
| (Rat_TSLPR- | LSVAIVCHDLESVEVTWGPGSAHHGLSANLSLE |
| aSym2) | FRYGNQVPQPCPHYFLLDSVRAGCVLPMGKGLL |
| | EVVLREGGGAKLFSRKKKASAWLRPRPPWNVTL |
| | SWVGDTVAVSCPSHSYPGLEYEVQHRDDFDPEW |
| | QSTSAPFCNLTVGGLDPGRCYDFRVRATPQDFY |
| | YGPEARPSKWTGVASLQGVGPTGSCTGPTLPRT |
| | PGTPTPPGGGGSGGGGSPEAIKAAAELGCAGIS |
| | SEEILELLRAAHELGLDPEAIKAAAELGKAGIS |
| | SEEILELLRAAHELGLDPEAIKAAAELGKAGIS |
| | SEEILELLRAAHELGLDPEAIKAAAELGKAGIS |
| | SEEILELLRAAHELGLDPEAIKAAAELGKAGIS |
| | SEEILELLRAAHELGLDPEAIKAAACLGKAGIS |
| | SEEILELLRAAHELGLGGGS |
| | (SEQ ID NO: 25) |
| MIDT-001744 | MGRLVLLWGAAVFLLGSWMALGQVATGEGLQIQ |
| (Cyno_TSLPR- | IMYFNLETVQVTWNASHYPRSNLSFHYKFSRDE |
| aSym3-sFLAG) | AYDQCTVYILQEGHTSGCLLDAQQQDDILYFSI |
| | RNGTHPVFTASRWIFYYLKPSSPKQVSFSWHQD |
| | AVTLTCSDLSYRGLLYEVQYRSPFDTEWQSKQE |
| | NTCNVTIEDLDAEKCYAFRARVKAMEDAYGPDT |
| | YPSDWSEVTCWQRGKTRDSCPEPRTPPKPKLSK |
| | GGGGSGGGGSPEAIKAACELGKAGISSEEILEL |
| | LRAAHELGLDPEAIKAAAELGKAGISSEEILEL |
| | LRAAHELGLDPEAIKAAAELGKAGISSEEILEL |
| | LRAAHELGLDPEAIKAAAELGKAGISSEEILEL |
| | LRAAHELGLDPEAIKAAAELGKAGISSEEILEL |
| | LRAAHELGLDPEAIKAAAELGKAGISSECILEL |
| | LRAAHELGLGGGSDYKDE |
| | (SEQ ID NO: 26) |
| MDT-001745 | MGRLVLLWGAAVFLLGGWMALGQGGAAEGVQIQ |
| (Human_TSLPR- | IIYFNLETVQVTWNASKYSRTNLTFHYRENGDE |
| aSym4- | AYDQCTNYLLQEGHTSGCLLDAEQRDDILYFSI |
| StrepII) | RNGTHPVFTASRWMVYYLKPSSPKHVRESWHQD |
| | AVTVTCSDLSYGDLLYEVQYRSPFDTEWQSKQE |
| | NTCNVTIEGLDAEKCYSFWVRVKAMEDVYGPDT |
| | YPSDWSEVTCWQRGEIRDACAETPTPPKPKLSK |
| | GGGGSGGGGSPEAIKAAAELGKAGISCEEILEL |
| | LRAAHELGLDPEAIKAAAELGKAGISSEEILEL |
| | LRAAHELGLDPEAIKAAAELGKAGISSEEILEL |
| | LRAAHELGLDPEAIKAAAELGKAGISSEEILEL |
| | LRAAHELGLDPEAIKAAAELGKAGISSEEILEL |
| | LRAAHELGLDPECIKAAAELGKAGISSEEILEL |
| | LRAAHELGLGGGSSAWSHPQFEK |
| | (SEQ ID NO: 27) |

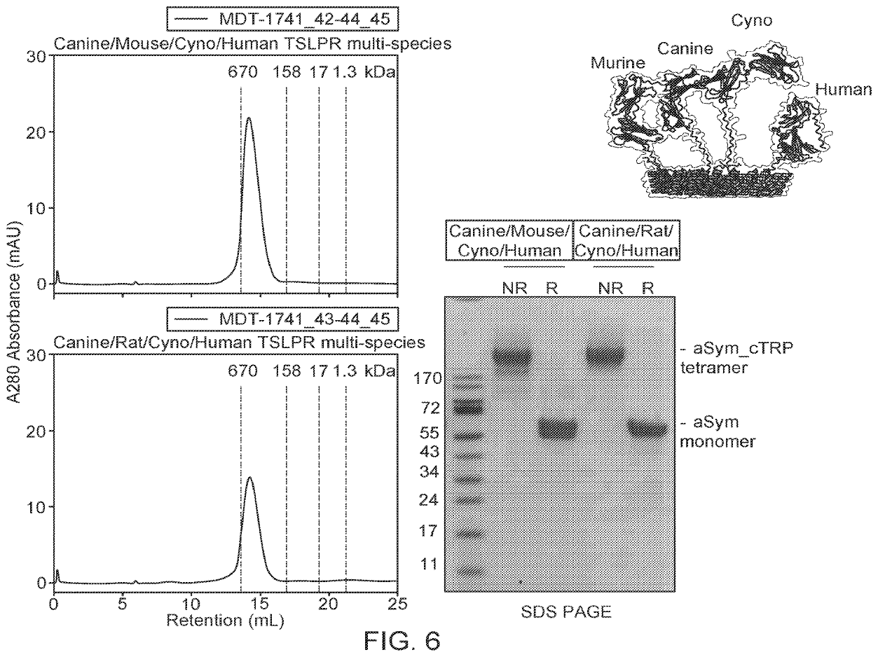

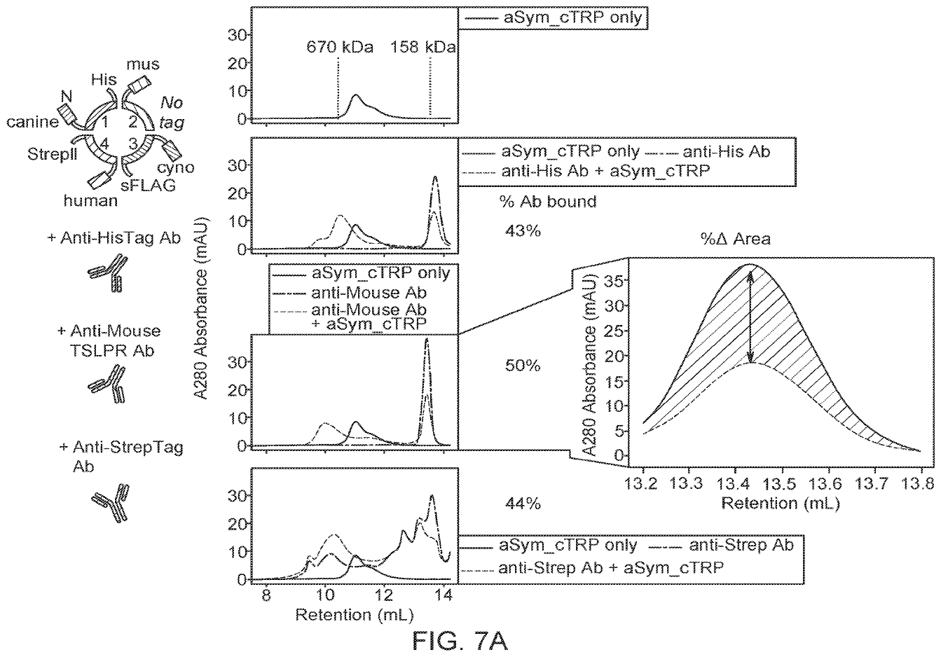

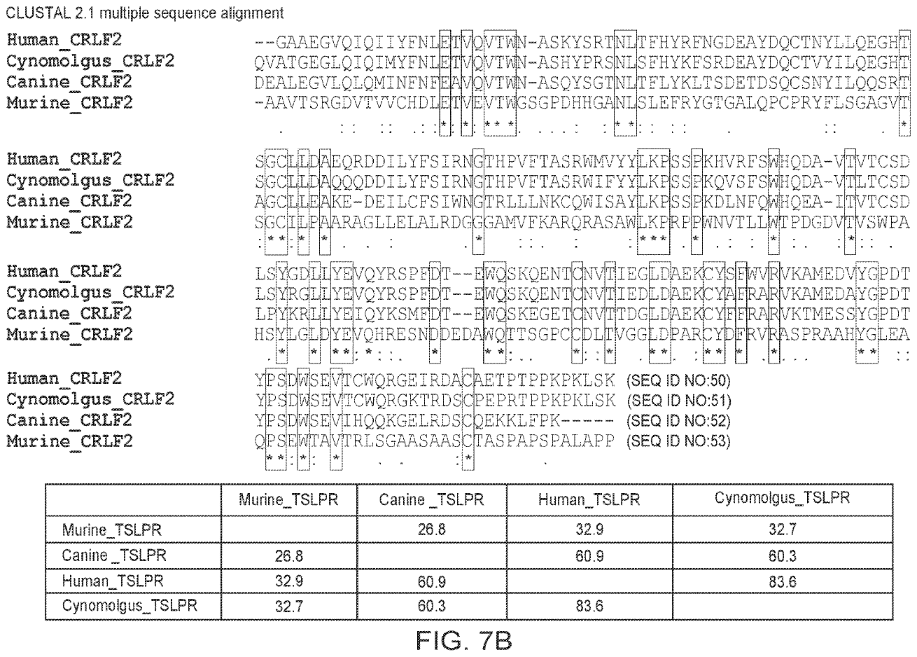

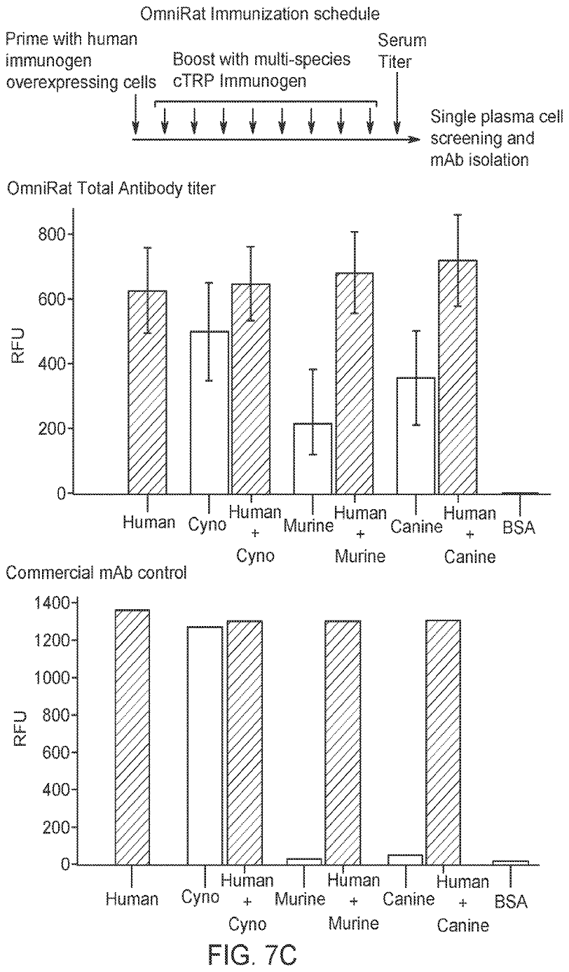

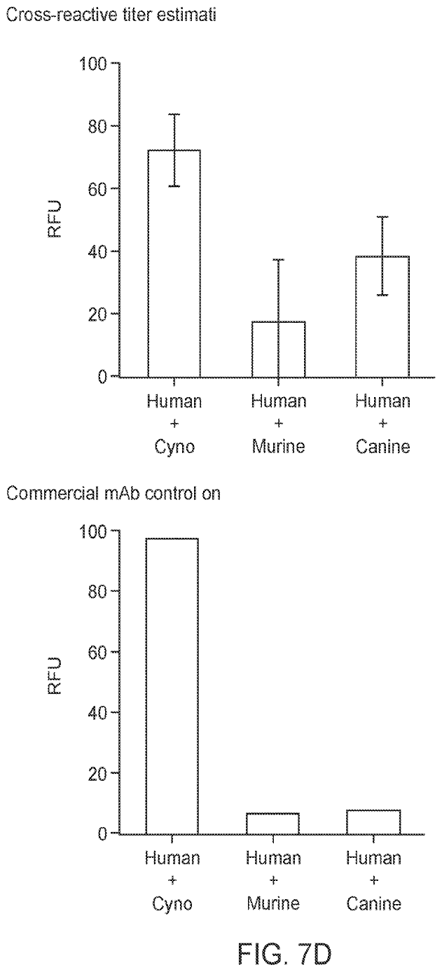

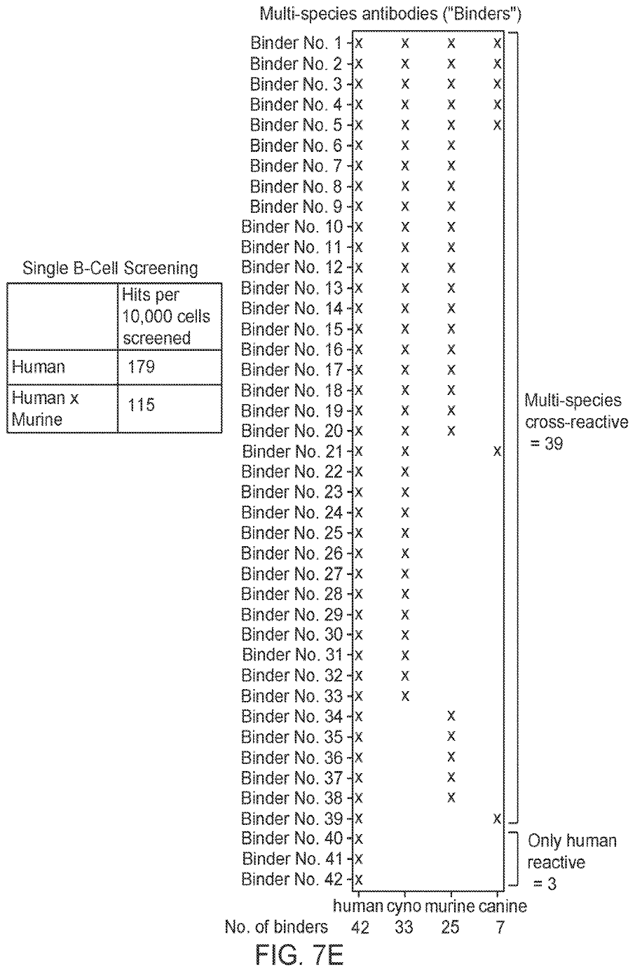

In each case, the full intact tetrameric immunogen was successfully produced by mammalian cells that were multiply transduced with each of individual genes. The proteins are monodisperse by size exclusion chromatography and the correct molecular weight by SDS PAGE (FIG. 6). The protomers self-assemble into a defined tetramer carrying an antigen from each species as was confirmed by an assay that probed the presence of each protomer by specific binding (FIG. 7A). The resulting immunogen can be used to amplify B cell responses that produce rare binders against all displayed antigens.

### Example 5. Application of Asymmetrical cTRPs-Multi-Species PDL1 Immunogens

In one example the asymmetric cTRP is used to present four PDL1 ectodomains from different species. The species may be human, rat, murine, cynomolgus or canine (FIG. 11). The assemblies may attach the ectodomain cargo with linkers of any length and type to the N or C termini. The protomers are expressible from cells that have been multiply transduced and form full oligomers in cell culture. The full intact protein can be readily purified from cell culture supernatant and is the correct molecular weight by size exclusion chromatography and SDS PAGE (FIGS. 12A-12B). In some embodiments, all genes are transduced concurrently into the same cell. In some embodiments, all genes are transduced sequentially into the same cell. In some embodiments, protomers self-assemble within the cell. In some embodiments, protomers self-assemble in solution. In other embodiments, the genes are transduced into independent cells to produce protomers individually, and then combined to form the full cTRP.

The resulting product can be injected as an immunogen that may cluster B cell receptors on the surface of B cells that bind conserved epitopes across the species PDL1 ectodomains. Selective engagement and expansion of these B cells may bias the antibody response towards interspecies cross reactivity.

### Example 6. Application of Asymmetrical cTRPs-T-Cell Expansion Reagents

T cell expansion requires clustering and engagement of multiple receptors. The asymmetric cTRP scaffold is particularly well suited for this application as it satisfies both requirements. In one example, an asymmetric cTRP presents multiple CD3 engaging domains (e.g., CD3 mAbs such as clone OKT3, SP34) and co-stimulatory engaging domains targeting co-stimulatory proteins (e.g., CD28, CD27, 4-1BB, OX40, ICOS or any other). Other examples include using any combination or exclusion of any of the stimulatory receptors. In this way, T cell populations are expanded ex vivo for adoptive T cell therapy or as a routine T cell expansion reagent for research.

### Example 7. Application of Asymmetrical cTRPs-Hetero-CAR T Cell Expansion and Screening

T cells are altered to present chimeric antigen receptors bypassing the requirement for TCR engagement to induce cytotoxicity. A given T cell is repeatedly transformed to present multiple distinct CARs on its surface. Detection of the CARs is complicated as their abundance is low, requiring highly avid staining reagents. To address this, a cTRP presenting the ligand of the CAR is employed as a binder, and in combination with multicolored secondaries that detect CAR expression for use in monitoring transduction efficiency.

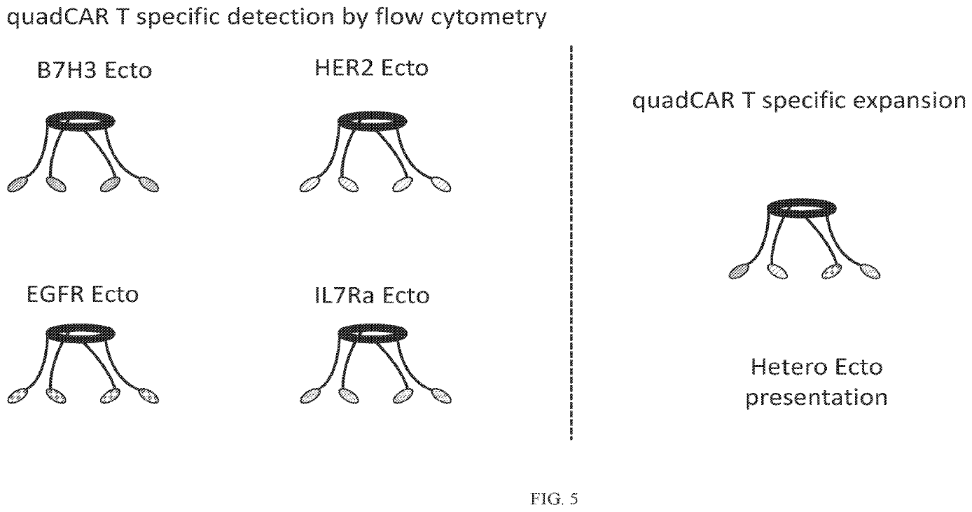

An exemplary hetero ecto presentation quad-cTRP is provided in FIG. 5, using B7H3 ecto, Her2 ecto, EGFR ecto, IL17Ra ecto. Alternatively, hetero ecto presentation cTRP that are dual, tri and hexo-hetero cTRPs are made.

### Example 8. Application of Asymmetrical cTRPs-Quadrivalent Flu Vaccine

Season to season heterogeneity of flu strain has hampered effectiveness of flu vaccines requiring bespoke annual vaccination. The primary cause of seasonal change is the hypermutation of the head of hemagglutinin (HA), the glycoprotein responsible for viral-host binding. An ideal flu vaccine would amplify a broadly neutralizing response where areas prone to greatest mutation on the antigen are not immunodominant. A multivalent flu vaccine where more than one strain of HA is presented in defined stoichiometry may be able to give rise to such an immune response. In this example, HA trimers are fused to the asymmetric cTRP so that only B cells with B cell receptors that interact in a multivalent way will undergo activation and expansion. This may produce a durable and broadly neutralizing humoral response. This platform can be used to present multiple of the same immunogens but of various strains that can generate broadly neutralizing antibodies. In some embodiments, HA trimers are enzymatically conjugated to the asymmetric cTRP. In some embodiments, monomeric HA immunogens are presented on asymmetric cTRP through recombinant production/direct genetic fusion.

Proof-of-Concept Asymmetric cTRP Sequences

Proof-of-concept asymmetric cTRP24(6) protomers were designed with unique candidate cysteine mutation pairs termed aSym1-4. (FIG. 13). Each protomer contains a furin cleavage site (SEQ ID NO:39) that cleaves as the protein is expressed into the supernatant exposing a tri-glycine sequence which is recognized for conjugation by our mammalian expressed sortase enzyme. Each protomer also contains varied affinity tags to quantify level of expression independently as well as quantification of asym cTRP formation; the affinity tags including sFlag (SEQ ID NO:38), HisTag (SEQ ID NO:40), and Strep-Tag (SEQ ID NO:41).

The asym cTRP 1-4 is co-expressed in HEK293F cells using the Daedalus mammalian expression platform. The harvested supernatant from the expression culture is purified by Ni-NTA affinity chromatography followed by size exclusion chromatography (SEC). Alternatively, asym cTRP 1-4 is expressed individually in HEK293F cells using the Daedalus mammalian expression platform and each component is purified independently dependent on the affinity tag, then combined in equimolar ratios where the asym cTRP will spontaneously form.

The ability to form asym cTRPs is herein demonstrated, as well as display cargo off asymmetrical cTRP. This indicated that presentation of Influenza HA strains on asym cTRP is feasible.

The design strategy used to generate asym cTRPs with furin cleavage sites for sortase enzyme conjugation indicates this approach could be applicable to the successful presentation of other viral immunogens to achieve broadly neutralizing vaccine candidates.

### Proof-of-Concept Influenza HA Sequences

Proof-of-concept Influenza A virus hemagglutinin (HA) sequences were designed of four strains in 2017-2018 seasonal influenza vaccines fused to a fibritin foldon trimerization domain. Each HA sequence contains a LPETG (SEQ ID NO:43) amino acid sequence at the C-terminus that is recognized by our mammalian expressed sortase enzyme. Each sequence also contains a 6×histidine tag (SEQ ID NO:40) for affinity purification.

The Influenza HA foldon trimers are expressed in HEK293F cells using the Daedalus mammalian expression platform. The harvested supernatant from the expression culture is purified by Ni-NTA affinity chromatography followed by size exclusion chromatography (SEC). The constructs can then be mixed in equimolar ratios with one of the four asym cTRP 1/2/3/4 protomers and with sortase enzyme, where sortase enzymatically links a asym cTRP protomer to HA foldon trimers, and the four HA foldon trimer-linked protomers are mixed and further assemble into a cTRP, generating a first-in-class quadrivalent vaccine candidate.

Mammalian expressed sortase enzyme recognizes an N-terminal tri-glycine sequence (“GGG”) and a C-terminal LPXTG (SEQ ID NO:44) sequence (e.g., LPETG (SEQ ID NO:43)), and conjugates them into an “LPXTGGG” (SEQ ID NO:45) linker.

The hemagglutinin (HA) of influenza virus is a homotrimeric integral membrane glycoprotein. The design strategy disclosed herein presents an influenza HA sequence as a trimer using a foldon and displays it on asym cTRP protomers, to form an assemblage capable of displaying two or more influenza HA trimer subtypes. This design strategy is conceived to be applicable to the successful presentation of other viral immunogens to achieve broadly neutralizing vaccine candidates.

### Proof-of-Concept Sortase Conjugation

Sortase was expressed in a mammalian system, characterized by using SDS-PAGE in reducing and nonreducing conditions (FIG. 15B), then tested for the conjugation activity (FIG. 15C).

Sortase was produced in HEK293F cells using the Daedalus mammalian expression system. The harvested supernatant from the expression culture was purified using Ni-NTA affinity chromatography followed by size exclusion chromatography (FIG. 15A). The purified material was characterized via SDS-PAGE (FIG. 15B) and assessed for its ability to conjugate a tri-glycine GFP fused protein to a VHH protein with an LPETG (SEQ ID NO:43) sequence (FIG. 15C).

It was possible to successfully produce, purify, and characterize activity of our mammalian expressed sortase. These results indicate that any two proteins with the corresponding sequences (LPETG (SEQ ID NO:43) and GGG) are recognized by our sortase and conjugated together.

These results indicate an alternative method of conjugation where any two constructs can be linked together given the presence of sequences recognized by sortase.

### Example 9. Application of Asymmetrical cTRPs-Substrate Channeling

Combination of or conversion from easily synthesized precursor molecules to more complex and industrially relevant molecules is an area of great consequence. Catalysts are required for production of most widely available chemical and medical products. Enzymatic catalysts have been explored in some manufacturing processes, however their usefulness has been limited in that at most three enzymes have been successfully linked into a single complex. Clustering of enzymes is necessary as it greatly enhances throughput by channeling the substrate from one enzyme to the next. Further, assembly into a complex reduces the dissipation of intermediates that may interact with each other. In this example, an asymmetric cTRP is used as a scaffold onto which several enzymes are fused. The resulting complex holds more than three enzymes, unlocking a new class of complexity in catalysis.

Although the foregoing specification and examples fully disclose and enable the present invention, they are not intended to limit the scope of the invention, which is defined by the claims appended hereto.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A protomer comprising the following formula: 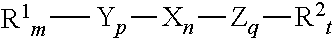
wherein
R¹ is an N-terminal linker or a covalent bond,
R² is a C-terminal linker or a covalent bond,
X is an α-helical peptide repeat unit comprising 30-35 amino acid residues and forming an outer α-helix and an inner α-helix joined by a helix-linker,
Y is a repeat unit having an identical amino acid sequence to X except for the substitution of one residue with a cysteine or with a non-canonical amino acid, or Y is a repeat unit having at least 90% sequence identity to X except for the substitution of the one residue with a cysteine or with a non-canonical amino acid and further except for C-terminal deletion or substitution of an amino acid residue outside of the outer α-helix—helix linker—inner α-helix,
Z is a repeat unit having an identical amino acid sequence identity to X except for the substitution of one residue with a cysteine or with a non-canonical amino acid, or Z is a repeat unit having at least 90% sequence identity to X except for the substitution of the one residue with a cysteine or with a non-canonical amino acid and further except for C-terminal deletion or substitution of an amino acid residue outside of the outer α-helix—helix linker—inner α-helix,
wherein the cysteine or the non-canonical amino acid in Z is at a different position than in Y, wherein if the substitution in Y is with the cysteine then the substitution in Z is with the cysteine, and wherein if the substitution in Y is with the non-canonical amino acid then the substitution in Z is with the non-canonical amino acid,
m is 0 or 1, wherein if m is 0 then R¹ is absent,
p is 1,
q is 1,
t is 0 or 1, wherein if t is 0 then R² is absent, and
n is a repeat number of the α-helical peptide repeat unit being 4, 1, 2, 6, 10 or 0.

2. The protomer of claim 1, wherein X is selected from the group consisting of the sequences of SEQ ID NOs: 7 and 54-553; or wherein X is PEAIKAAAELGKA (SEQ ID NO:15)—a first GBB linker—SEEILELLRAAHEL (SEQ ID NO: 16)—a second GBB linker, wherein the first and the second GBB linkers are independently selected from GIS; GLD; GKS; GIT; GTT; GYS; GDK; GOE; NDK; GDR; or GDL; or wherein X has at least 90% or 95% identity to PEAIKAAAELGKAGISSEEILELLRAAHELGLD (SEQ ID NO:7); and/or wherein the substitutions of the one amino acid residue with the cysteine or with the non-canonical amino acid in Y and in Z are not at amino acid position 1 and position 3 of the outer α-helix—helix linker—inner α-helix.

3-8. (canceled)

9. The protomer of claim 1, wherein R¹ and R² are independently selected from the group consisting of GGS, GGGS (SEQ ID NO:5), GGSGGGSGGSG (SEQ ID NO:17), GGSGGGSGSG (SEQ ID NO:18), GGSGGGSG (SEQ ID NO:19), GGGGSGGGGS (SEQ ID NO:20), GGGSGGGS (SEQ ID NO:21), and GGSGGS (SEQ ID NO:22), and wherein m is 1 and t is 1 when R¹ and R² are selected, respectively.

10. (canceled)

11. The protomer of claim 1, wherein the substitution in Y and the substitution in Z are with cysteine; and wherein Y has at least 90% or 95% identity to CEAIKAAAELGKAGISSEEILELLRAAHELGLD (SEQ ID NO:6), p is 1, and the first amino acid residue is C, or wherein Y is CEAIKAAAELGKAGISSEEILELLRAAHELGLD (SEQ ID NO:6), and p is 1.

12. (canceled)

13. (canceled)

14. The protomer of claim 11, wherein Z has at least 90% or 95% identity to PEAIKAAAELGKCGISSEEILELLRAAHELGL (SEQ ID NO: 8), q is 1, and the 13^{th} amino acid residue is a cysteine; or wherein Z is PEAIKAAAELGKCGISSEEILELLRAAHELGL (SEQ ID NO:8), and q is 1.

15. (canceled)

16. (canceled)

17. The protomer of claim 1, wherein
Y is CEAIKAAAELGKAGISSEEILELLRAAHELGLD (SEQ ID NO:6), and p is 1,
X is PEAIKAAAELGKAGISSEEILELLRAAHELGLD (SEQ ID NO:7) and n is 4, and
Z is PEAIKAAAELGKCGISSEEILELLRAAHELGL (SEQ ID NO:8), and q is 1.

18. The protomer of claim 1, wherein the substitution in Y and the substitution in Z are with cysteine; and wherein Y has at least 90% or 95% identity to PEAIKAAAELGCAGISSEEILELLRAAHELGLD (SEQ ID NO:9), q is 1, and the 12^{th} amino acid residue is a cysteine, or wherein Y is PEAIKAAAELGCAGISSEEILELLRAAHELGLD (SEQ ID NO:9), and q is 1.

19. (canceled)

20. (canceled)

21. The protomer of claim 18, wherein Z has at least 90% or 95% identity to PEAIKAAACLGKAGISSEEILELLRAAHELGL (SEQ ID NO: 10), q is 1, and the ninth amino acid is a cysteine; or wherein Z is PEAIKAAACLGKAGISSEEILELLRAAHELGL (SEQ ID NO:10), and q is 1.

22. (canceled)

23. (canceled)

24. The protomer of claim 1, wherein
Y is PEAIKAAAELGCAGISSEEILELLRAAHELGLD (SEQ ID NO:9), and p is 1,
X is PEAIKAAAELGKAGISSEEILELLRAAHELGLD (SEQ ID NO:7) and n is 4, and
Z is PEAIKAAACLGKAGISSEEILELLRAAHELGL (SEQ ID NO:10), and q is 1.

25. The protomer of claim 1, wherein the substitution in Y and the substitution in Z are with cysteine; and wherein Y has at least 90% or 95% identity to PEAIKAACELGKAGISSEEILELLRAAHELGLD (SEQ ID NO:11), p is 1, and the eighth amino acid is a cysteine, or wherein Y is PEAIKAACELGKAGISSEEILELLRAAHELGLD (SEQ ID NO:11), and p is 1.

26. (canceled)

27. (canceled)

28. The protomer of claim 25, wherein Z has at least 90% or 95% identity to PEAIKAAAELGKAGISSECILELLRAAHELGL (SEQ ID NO: 12), q is 1, and the nineteenth amino acid is a cysteine; or wherein Z is PEAIKAAAELGKAGISSECILELLRAAHELGL (SEQ ID NO:12), and q is 1.

29. (canceled)

30. (canceled)

31. The protomer of claim 1, wherein
Y is PEAIKAACELGKAGISSEEILELLRAAHELGLD (SEQ ID NO:11), and p is 1,
X is PEAIKAAAELGKAGISSEEILELLRAAHELGLD (SEQ ID NO:7) and n is 4, and
Z is PEAIKAAAELGKAGISSECILELLRAAHELGL (SEQ ID NO:12), and q is 1.

32. The protomer of claim 1, wherein the substitution in Y and the substitution in Z are with cysteine; and wherein Y has at least 90% or 95% identity to PEAIKAAAELGKAGISCEEILELLRAAHELGLD (SEQ ID NO:13), p is 1, and the seventeenth amino acid is a cysteine, or wherein Y is PEAIKAAAELGKAGISCEEILELLRAAHELGLD (SEQ ID NO:13), and p is 1.

33. (canceled)

34. (canceled)

35. The protomer of claim 32, wherein Z has at least 90% or 95% identity to PECIKAAAELGKAGISSEEILELLRAAHELGL (SEQ ID NO: 14), q is 1, and the third amino acid is a cysteine; or wherein Z is PECIKAAAELGKAGISSEEILELLRAAHELGL (SEQ ID NO:14), and q is 1.

36. (canceled)

37. (canceled)

38. The protomer of claim 1, wherein
Y is PEAIKAAAELGKAGISCEEILELLRAAHELGLD (SEQ ID NO:13), and p is 1,
X is PEAIKAAAELGKAGISSEEILELLRAAHELGLD (SEQ ID NO:7) and n is 4, and
Z is PECIKAAAELGKAGISSEEILELLRAAHELGL (SEQ ID NO:14), and q is 1.

39. The protomer of claim 1, wherein a cargo molecule is operably linked to R¹ or R², and wherein the cargo molecule is a microbe or a microbial subunit, a protein, a small molecule ligand binding domain, or a nucleic acid,
wherein the microbe comprises a virus, optionally the virus comprising influenza, optionally the influenza being Influenza HA,
wherein the nucleic acid comprises a short hairpin RNA (shRNA), an siRNA, an antisense oligonucleotide, or a component of CRISPR/CAS9 or other gene editing molecules,
wherein the protein comprises an immunogen, a cytokine, an enzyme, or a B cell receptor ligand, optionally the immunogen being TSLPR or CD3, optionally the TSLPR immunogen being MIDT-001741 (Canine_TSLPR-aSym1-His; SEQ ID NO:23), MDT-001742 (Murine_TSLPR-aSym2; SEQ ID NO:24), MDT-001743 (Rat_TSLPR-aSym2; SEQ ID NO:25), MDT-001744 (Cyno_TSLPR-aSym3-Sflag; SEQ ID NO:26) or MDT-001745 (Human_TSLPR-aSym4-StrepII; SEQ ID NO:27),
optionally the cargo molecule being a VHH binder, a human thymic stromal lymphopoietin receptor (TSLPR) ectodomain, or PD-L1, a cytokine for T cell expansion, a Notch ligand an immunogenic peptide, a peptide adjuvant, a single-chain class I MHC domain, a peptide binding domain, phosphotyrosine binding domain, LIM domain, SAM domain, PDZ domain, FERM domain, Pleckstrin homology domain, VVVV domain, oligosaccharide binding (OB) domain, Immunoglobulin domain (IgG), single chain MHC protein, tumor specific antigen (optionally CD19 or ROR1), isolated antigenic or adjuvant peptide, small molecule ligand binding domain (e.g., maltose binding protein) and enzyme domain (optionally trypsin), SH2, Protein L, cytokines (optionally IL-2; IL-17c, IL-3); Notch ligands (optionally Delta or Jagged); immunogenic peptides; peptide adjuvants; single chain variable fragment nanobodies (Nbs); viral, prokaryotic or eukaryotic immunogen; or an individual member of a catalytic pathway,
optionally the cargo molecule being a human, rat, murine cynomolgus or canine PDL1 ectodomain,
optionally the cargo molecule being a T-cell expansion reagent or a T cell engaging protein that synergistically activates T cells with co-presented cargo,
optionally the cargo molecule being a CD3 engaging domain,
optionally the cargo molecule being a co-stimulatory domain,
optionally the cargo molecule being CD28, CD27, 4-1BB, OX40, or ICOS,
optionally the cargo molecule being a ligand of chimeric antigen receptor (CAR) or being an antigen recognized by CAR to specifically activate CAR T cells that recognize all presented antigens,
optionally the cargo molecule being B7H3 ecto, Her2 ecto, EGFR ecto, or IL17Ra ecto,
optionally the cargo molecule being a homologous protein immunogen from a different species selected to specifically activate B cells that recognize all presented immunogens,
optionally the cargo molecule comprising an influenza viral glycoprotein immunogen from different strains to specifically activate B cells that recognize all presented strains, optionally the influenza viral glycoprotein immunogen being fused with a trimerization domain,
optionally the cargo molecule being a substrate channeling enzyme, wherein the substrate channeling enzyme comprises a recognition motif LPXTG (SEQ ID NO:44) at the c-terminus and pentaglycine, triglycine, or diglycine motif on the N-terminus.

40-69. (canceled)

70. An asymmetrical circular tandem repeat protein (cTRP) comprising:
(a) four protomers, associated in tandem in a closed circular structure, wherein each protomer independently comprises the following formula: 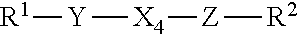
wherein
R¹ is an N-terminal linker or a covalent bond,
R² is a C-terminal linker or a covalent bond,
X is an α-helical peptide repeat unit comprising 30-35 amino acid residues and forming an outer α-helix and an inner α-helix joined by a helix-linker, and X being devoid of cysteines,
Y is a repeat unit having an identical amino acid sequence or at least 90% sequence identity to X except for the substitution of one residue with a cysteine or a non-canonical amino acid, and
Z is a repeat unit having an identical amino acid sequence or at least 90% sequence identity to X except for the substitution of one residue with a cysteine or a non-canonical amino acid, wherein the cysteine or the non-canonical amino acid in Z is at a different position than in Y, wherein if the substitution in Y is with the cysteine then the substitution in Z is with the cysteine, and wherein if the substitution in Y is with the non-canonical amino acid then the substitution in Z is with the non-canonical amino acid;
wherein the cysteine residue or the non-canonical amino acid residue in Z of each protomer forms a covalent bond with the cysteine residue or the non-canonical amino acid residue, respectively, in Y of a next protomer in tandem in the closed circular structure;
wherein the protein is left-handed; and
wherein each pair of cysteine substitutions in Z of a protomer and Y of a next protomer is different in amino acid residue positions relative to respective protomers, and wherein each pair of non-canonical amino acid residue substitutions in Z of a protomer and Y of a next protomer is a mutually exclusive orthogonal pair of reactive groups for click conjugation;
(b) two protomers, associated in tandem in a closed circular structure, wherein each protomer independently comprises the following formula: 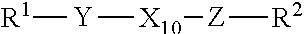
wherein
R¹ is an N-terminal linker or a covalent bond,
R² is a C-terminal linker or a covalent bond,
X is an α-helical peptide repeat unit comprising 30-35 amino acid residues and forming an outer α-helix and an inner α-helix joined by a helix-linker, X being devoid of cysteines,
Y is a repeat unit having an identical amino acid sequence or at least 90% sequence identity to X except for the substitution of one residue with a cysteine or with a non-canonical amino acid, and
Z is a repeat unit having an identical amino acid sequence or at least 90% sequence identity to X except for the substitution of one residue with a cysteine or with a non-canonical amino acid, wherein the cysteine or the non-canonical amino acid in Z is at a different position than in Y, wherein if the substitution in Y is with the cysteine then the substitution in Z is with the cysteine, and wherein if the substitution in Y is with the non-canonical amino acid then the substitution in Z is with the non-canonical amino acid;
wherein the cysteine or the non-canonical amino acid residue in Z of each protomer forms a covalent bond with the cysteine or the non-canonical amino acid residue, respectively, in Y of a next protomer in tandem in the closed circular structure;
wherein the protein is left-handed; and
wherein each pair of cysteine substitutions in Z of a protomer and Y of a next protomer is different in amino acid residue positions relative to respective protomers, and wherein each pair of non-canonical amino acid residue substitutions in Z of a protomer and Y of a next protomer is an orthogonal pair of reactive groups for click conjugation;
(c) three protomers, associated in tandem in a closed circular structure, wherein each protomer independently comprises the following formula: 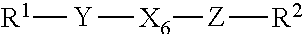
wherein
R¹ is an N-terminal linker or a covalent bond,
R² is a C-terminal linker or a covalent bond,
X is an α-helical peptide repeat unit comprising 30-35 amino acid residues and forming an outer α-helix and an inner α-helix joined by a helix-linker, and X being devoid of cysteines,
Y is a repeat unit having an identical amino acid sequence or at least 90% sequence identity to X except for the substitution of one residue with a cysteine or with a non-canonical amino acid, and
Z is a repeat unit having an identical amino acid sequence or at least 90% sequence identity to X except for the substitution of one residue with a cysteine or with a non-canonical amino acid, respectively, wherein the cysteine or the non-canonical amino acid in Z is at a different position than in Y, wherein if the substitution in Y is with the cysteine then the substitution in Z is with the cysteine, and wherein if the substitution in Y is with the non-canonical amino acid then the substitution in Z is with the non-canonical amino acid;
wherein the cysteine residue or the non-canonical amino acid residue in Z of each protomer forms a covalent bond with the cysteine or the non-canonical amino acid residue, respectively, in Y of a next protomer in tandem in the closed circular structure;
wherein the protein is left-handed; and
wherein each pair of cysteine substitutions in Z of a protomer and Y of a next protomer is different in amino acid residue positions relative to respective protomers, and wherein each pair of non-canonical amino acid residue substitutions in Z of a protomer and Y of a next protomer is an orthogonal pair of reactive groups for click conjugation;
or
(d) six protomers, associated in tandem in a closed circular structure, wherein each protomer independently comprises the following formula: 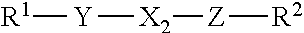
wherein
R¹ is an N-terminal linker or a covalent bond,
R² is a C-terminal linker or a covalent bond,
X is an α-helical peptide repeat unit comprising 30-35 amino acid residues and forming an outer α-helix and an inner α-helix joined by a helix-linker, X being devoid of cysteines,
Y is a repeat unit having an identical amino acid sequence or at least 90% sequence identity to X except for the substitution of one residue with a cysteine or with a non-canonical amino acid, and
Z is a repeat unit having an identical amino acid sequence or at least 90% sequence identity to X except for the substitution of one residue with a cysteine or with a non-canonical amino acid, wherein the cysteine or the non-canonical amino acid in Z is at a different position than in Y, wherein if the substitution in Y is with the cysteine then the substitution in Z is with the cysteine, and wherein if the substitution in Y is with the non-canonical amino acid then the substitution in Z is with the non-canonical amino acid;
wherein the cysteine or the non-canonical amino acid residue in Z of each protomer forms a covalent bond with the cysteine or the non-canonical amino acid residue, respectively, in Y of a next protomer in tandem in the closed circular structure;
wherein the protein is left-handed; and
wherein each pair of cysteine substitutions in Z of a protomer and Y of a next protomer is different in amino acid residue positions relative to respective protomers, and wherein each pair of non-canonical amino acid residue substitutions in Z of a protomer and Y of a next protomer is an orthogonal pair of reactive groups for click conjugation;
or
(e) 12 protomers, associated in tandem in a closed circular structure, wherein each protomer independently comprises the following formula: 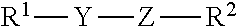
wherein
R¹ is an N-terminal linker or a covalent bond,
R² is a C-terminal linker or a covalent bond,
Y is an α-helical peptide repeat unit comprising 30-35 amino acid residues and comprises a single, cysteine or non-canonical amino acid, and Z is an α-helical peptide repeat unit comprising 30-35 amino acid residues and comprises a single, cysteine or non-canonical amino acid, respectively,
wherein the cysteine or the non-canonical amino acid residue in Z of each protomer forms a covalent bond with the cysteine or the non-canonical amino acid residue, respectively, in Y of a next protomer in tandem in the closed circular structure;
wherein the protein is left-handed; and
wherein each pair of cysteine substitutions in Z of a protomer and Y of a next protomer is different in amino acid residue positions relative to respective protomers, and wherein each pair of non-canonical amino acid residue substitutions in Z of a protomer and Y of a next protomer is an orthogonal pair of reactive groups for click conjugation.

71. The cTRP of claim 70, wherein the substitutions in pairs of Z of a protomer and Y of a next protomer in the closed circular structure are at:
position 1 and position 33,
position 1 and position 2,
position 1 and position 3,
position 1 and position 32,
position 2 and position 33,
position 3 and position 33,
position 3 and position 32,
position 4 and position 3,
position 4 and position 6,
position 12 and position 6,
position 8 and position 9,
position 8 and position 10,
position 8 and position 13,
position 8 and position 6,
position 11 and position 10,
position 11 and position 13,
position 11 and position 15,
position 12 and position 13,
position 17 and position 19,
position 21 and position 23,
position 28 and position 30,
position 4 and position 27,
position 11 and position 15, or
position 4 and position 33,
relative to respective protomers, wherein the amino acid numbering is based on amino acid positions in or aligned with PEAIKAAAELGKA (SEQ ID NO:15)—GBB linker—SEEILELLRAAHEL (SEQ ID NO:16)—GLD, wherein the GBB linker comprises GIS; GKS; GIT; GTT; GYS; GDK; GOE; NDK; GDR; or GDL.

72. The cTRP of claim 70, wherein X of each promoter is a same peptide selected from the group consisting of the sequences in FIGS. 8A-8V, wherein Z and Y have an identical amino acid sequence as X except for the substitutions and optionally a deletion or substitution of a C-terminal amino acid residue outside of the outer and inner α-helices, and wherein the substitutions of Z of a protomer and Y of a next protomer in the closed circular structure are a pair at:
position 1 and position 33,
position 1 and position 2,
position 1 and position 3,
position 1 and position 32,
position 2 and position 33,
position 3 and position 33,
position 3 and position 32,
position 4 and position 3,
position 4 and position 6,
position 12 and position 6,
position 8 and position 9,
position 8 and position 10,
position 8 and position 13,
position 8 and position 6,
position 11 and position 10,
position 11 and position 13,
position 11 and position 15,
position 12 and position 13,
position 17 and position 19,
position 21 and position 23,
position 28 and position 30,
position 4 and position 27,
position 11 and position 15, or
position 4 and position 33,
wherein the positions are amino acid residue positions in Z and Y of adjacent protomers.

73. The cTRP of claim 70, wherein R¹ is an N-terminal linker, and/or R² is a C-terminal linker, or wherein R1 is GGG or pentaglycine or diglycine, and R2 is an amino acid sequence Sortase recognition motif sequence LPXTG (SEQ ID NO:44); and wherein a cargo molecule is operably linked to R¹ and/or R², optionally the cTRP comprising at least two different cargo molecules operably linked to the R¹ and R²;
wherein the cargo molecule is a microbe or a microbial subunit, a protein, a small molecule ligand binding domain, or a nucleic acid, or wherein the cargo molecule is a cytokine, a Notch ligand an immunogenic peptide, a peptide adjuvant, a single-chain class I MHC domain, or a small molecule ligand binding domain,
wherein the microbe comprises a virus, optionally the virus comprising influenza, optionally the influenza being Influenza HA,
wherein the nucleic acid comprises a short hairpin RNA (shRNA), an siRNA, an antisense oligonucleotide, or a component of CRISPR/CAS9 or other gene editing molecules,
wherein the protein comprises an immunogen, a cytokine, an enzyme, or a B cell receptor ligand, optionally the immunogens being from different species of organisms selected from the group consisting of mouse, canine, non-human primate, human, pig, and rat, optionally the immunogen being TSLPR or CD3, optionally the TSLPR immunogen being MDT-001741 (Canine_TSLPR-aSym1-His; SEQ ID NO:23), MIDT-001742 (Murine_TSLPR-aSym2; SEQ ID NO: 24), MIDT-001743 (Rat_TSLPR-aSym2; SEQ ID NO:25), MDT-001744 (Cyno_TSLPR-aSym3-Sflag; SEQ ID NO:26) or MIDT-001745 (Human_TSLPR-aSym4-StrepII; SEQ ID NO:27),
optionally the cargo molecule being a VHH binder, a human thymic stromal lymphopoietin receptor (TSLPR) ectodomain, or PD-L1, a cytokine for T cell expansion, a Notch ligand an immunogenic peptide, a peptide adjuvant, a single-chain class I MHC domain, a peptide binding domain, phosphotyrosine binding domain, LIM domain, SAM domain, PDZ domain, FERM domain, Pleckstrin homology domain, VVVV domain, oligosaccharide binding (OB) domain, Immunoglobulin domain (IgG), single chain MHC protein, tumor specific antigen (optionally CD19 or ROR1), isolated antigenic or adjuvant peptide, small molecule ligand binding domain (e.g., maltose binding protein) and enzyme domain (optionally trypsin), SH2, Protein L, cytokines (optionally IL-2; IL-17c, IL-3); Notch ligands (optionally Delta or Jagged); immunogenic peptides; peptide adjuvants; single chain variable fragment nanobodies (Nbs); viral, prokaryotic or eukaryotic immunogen; or an individual member of a catalytic pathway,
optionally the cargo molecule being a human, rat, murine cynomolgus or canine PDL1 ectodomain,
optionally the cargo molecule being a T-cell expansion reagent or a T cell engaging protein that synergistically activates T cells with co-presented cargo,
optionally the cargo molecule being a CD3 engaging domain,
optionally the cargo molecule being a co-stimulatory domain,
optionally the cargo molecule being CD28, CD27, 4-1BB, OX40, or ICOS, optionally the cargo molecule being a ligand of chimeric antigen receptor (CAR) or being an antigen recognized by CAR to specifically activate CAR T cells that recognize all presented antigens,
optionally the cargo molecule being B7H3 ecto, Her2 ecto, EGFR ecto, or IL17Ra ecto,
optionally the cargo molecule being a substrate channeling enzyme, wherein the substrate channeling enzyme comprises a recognition motif LPXTG (SEQ ID NO:44) at the c-terminus and pentaglycine, triglycine, or diglycine motif on the N-terminus.

74-105. (canceled)

106. An asymmetrical circular tandem repeat protein (cTRP) comprising four protomers,
wherein each of the protomers comprise a different one of cTRP24(6)SS_aSym1 (SEQ ID NO: 1), cTRP24(6)SS_aSym2 (SEQ ID NO: 2), cTRP24(6)SS_aSym3 (SEQ ID NO: 3) and cTRP24(6)SS_aSym4 (SEQ ID NO: 4),
wherein the protomers self-assemble to form a toroid, and
wherein disulfide bonds form between the protomers.

107. The asymmetrical cTRP of claim 106, wherein a first cargo molecule is operably linked to cTRP24(6)SS_aSym1 (SEQ ID NO: 1), a second cargo molecule is operably linked to cTRP24(6)SS_aSym2 (SEQ ID NO: 2), a third cargo molecule is operably linked to cTRP24(6)SS_aSym3 (SEQ ID NO: 3), and a fourth cargo molecule is operably linked to cTRP24(6)SS_aSym4 (SEQ ID NO: 4),
wherein the protein is left-handed, and/or
wherein at least two of the cargo molecules immunogens from different species of organisms, optionally the species of organisms being selected from the group consisting of mouse, canine, non-human primate, human, pig, and rat,
wherein the cargo molecule is operably linked to R1 or R2, optionally linked through click chemistry that uses mutually orthogonal binding pairs in click chemistry, or uses SPAAC, Tetrazine ligation, Bulky Tetrazine-isonitrile ligation, Boron+Nitrogen Heterocycle formation, SnO Oct+Type I/II dipole, Oxime ligation, Thiol-ene ligation, CuACC, Quadricyclane ligation, or photoligation,
wherein the cargo molecule is a cytokine, a Notch ligand an immunogenic peptide, a peptide adjuvant, a single-chain class I MHC domain, or a small molecule ligand binding domain, or wherein the cargo molecule is a microbe or a microbial subunit, a protein, a small molecule ligand binding domain, or a nucleic acid, wherein the microbe comprises a virus, optionally the virus comprising influenza, optionally the influenza being Influenza HA,
wherein the nucleic acid comprises a short hairpin RNA (shRNA),
an siRNA, an antisense oligonucleotide, or a component of CRISPR/CAS9 or other gene editing molecules,
wherein the protein comprises an immunogen, a cytokine, an enzyme, or a B cell receptor ligand, optionally the immunogen being TSLPR or CD3, optionally the TSLPR immunogen being MDT-001741 (Canine_TSLPR-aSym1-His; SEQ ID NO:23), MDT-001742 (Murine_TSLPR-aSym2; SEQ ID NO:24), MDT-001743 (Rat_TSLPR-aSym2; SEQ ID NO:25), MDT-001744 (Cyno_TSLPR-aSym3-Sflag; SEQ ID NO: 26) or MDT-001745 (Human_TSLPR-aSym4-StrepII; SEQ ID NO: 27),
optionally the cargo molecule being a VHH binder, a human thymic stromal lymphopoietin receptor (TSLPR) ectodomain, or PD-L1, a cytokine for T cell expansion, a Notch ligand an immunogenic peptide, a peptide adjuvant, a single-chain class I MHC domain, a peptide binding domain, phosphotyrosine binding domain, LIM domain, SAM domain, PDZ domain, FERM domain, Pleckstrin homology domain, VVVV domain, oligosaccharide binding (OB) domain, Immunoglobulin domain (IgG), single chain MHC protein, tumor specific antigen (optionally CD19 or ROR1), isolated antigenic or adjuvant peptide, small molecule ligand binding domain (e.g., maltose binding protein) and enzyme domain (optionally trypsin), SH2, Protein L, cytokines (optionally IL-2; IL-17c, IL-3); Notch ligands (optionally Delta or Jagged); immunogenic peptides; peptide adjuvants; single chain variable fragment nanobodies (Nbs); viral, prokaryotic or eukaryotic immunogen; or an individual member of a catalytic pathway,
optionally the cargo molecule being a human, rat, murine cynomolgus or canine PDL1 ectodomain,
optionally the cargo molecule being a T-cell expansion reagent or a T cell engaging protein that synergistically activates T cells with co-presented cargo,
optionally the cargo molecule being a CD3 engaging domain,
optionally the cargo molecule being a co-stimulatory domain,
optionally the cargo molecule being CD28, CD27, 4-1BB, OX40, or ICOS,
optionally the cargo molecule being a ligand of chimeric antigen receptor (CAR) or being an antigen recognized by CAR to specifically activate CAR T cells that recognize all presented antigens,
optionally the cargo molecule being B7H3 ecto, Her2 ecto, EGFR ecto, or IL17Ra ecto, and/or
optionally the cargo molecule being a substrate channeling enzyme, wherein the substrate channeling enzyme comprises a recognition motif LPXTG (SEQ ID NO:44) at the c-terminus and pentaglycine, triglycine, or diglycine motif on the N-terminus.

108-139. (canceled)

140. A vector comprising a nucleic acid molecule encoding a protomer of claim 1, optionally wherein the nucleic acid molecule further encodes one or more of a signal sequence at the N-terminus of the protomer, a sortase recognition motif, a fibritin foldon trimerization domain, and an affinity tag.

141. (canceled)

142. A method of generating monoclonal antibodies cross-protective against a microbe, the method comprising:
(a) isolating cells from a sample of blood or other tissue containing cells of the immune system from a subject that has been administered or infected with an asymmetrical circular tandem repeat protein (cTRP) of claim 70, wherein proteins of two or more strains or subtypes of the microbe are each operably linked to R¹ or R² of the cTRP;
(b) preparing monoclonal antibodies using the cells of (a); and
(c) selecting monoclonal antibodies that cross-react with different strains and subtypes of the microbe;
optionally wherein the microbe comprises influenza, and the proteins operably linked to R1 or R2 of the cTRP are hemagglutinins of two or more strains or subtypes of the influenza, or optionally wherein the proteins operably linked R1 or R2 of the cTRP are each fused with a fibritin foldon trimerization domain, trimerized, and then bonded to R1 or R2.

143. (canceled)

144. (canceled)

145. A method for inducing an immunogenic response to an immunogenic protein from two or more species or to two or more immunogenic proteins, the method comprising administering an asymmetrical circular tandem repeat protein (cTRP) of claim 70 to a subject in need thereof, wherein the immunogenic protein from two or more species or the two or more immunogenic proteins are each operably linked to R¹ or R² of the cTRP; and optionally wherein the immunogenic protein comprises PDL1 ectodomains or TSLPR ectodomains from two or more species.

146. (canceled)

147. A method of inducing T-cell expansion, comprising culturing T-cells in the presence of an asymmetrical circular tandem repeat protein (cTRP) of claim 70, wherein two or more CD3 engaging domains and one or more co-stimulatory domains are each operably linked to R¹ or R² of the cTRP, wherein the co-stimulatory domains comprise one or more of CD28, CD27, 4-1BB, OX40, and ICOS.

148. (canceled)

149. A method of inducing engineered T-cell expansion and/or detection, wherein the T-cell is engineered with one or more surface receptors that bind to one or more ligands, the method comprising culturing the engineered T-cell in the presence of an asymmetrical circular tandem repeat protein (cTRP) of claim 70, wherein the one or more ligands are each operably linked to R¹ or R² of the cTRP, wherein the ligands operably linked to R1 or R2 comprise ectodomains of one or more of B7H3, Her2, EGFR, and IL17Ra.

150. (canceled)

151. A method of channeling a substrate for reactions mediated by two or more enzymes, comprising contacting the substrate with an asymmetrical circular tandem repeat protein (cTRP) of claim 70, wherein the two or more enzymes are each operably linked to R¹ or R² of the cTRP.

152. A method of preparing an asymmetrical circular tandem repeat protein (cTRP) of claim 70, comprising:
expressing two or more vectors each comprising a polynucleic aid encoding a different protomer, thereby obtaining two more different protomers; and
mixing the two or more different protomers at an equal molar ratio, whereby the two or more different protomers form a closed circular structure, thereby obtaining an asymmetrical cTRP,
wherein the two or more different protomers each comprise a polypeptide having the formula of: 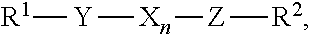
wherein
R¹ is an N-terminal linker or a covalent bond,
R² is a C-terminal linker or a covalent bond,
X is an α-helical peptide repeat unit comprising 30-35 amino acid residues and forming an outer α-helix and an inner α-helix joined by a helix-linker,
Y is a repeat unit having an identical amino acid sequence as X except for substitution of one residue with a cysteine or with a non-canonical amino acid and optionally further except for deletion or substitution of an amino acid residue at the terminus outside of the outer and inner α-helices,
Z is a repeat unit having an identical amino acid sequence as X except for substitution of one residue with a cysteine or with a non-canonical amino acid and optionally further except for deletion of an amino acid residue at the terminus outside of the outer and inner α-helices, wherein the cysteine or the non-canonical amino acid in Z is at a different position than in Y, wherein if the substitution in Y is with the cysteine then the substitution in Z is with the cysteine, and wherein if the substitution in Y is with the non-canonical amino acid then the substitution in Z is with the non-canonical amino acid, and
n is a repeat number of the α-helical peptide repeat unit being 0, 1, 2, 4, 6 or 10; and
wherein the substitutions with cysteines are at different amino acid positions in each Y and Z of the two or more different protomers, or wherein the substitutions with the non-canonical amino acids in each pair of Z of a protomer and Y of a next protomer are with mutually exclusive orthogonal pair of reactive residues for click conjugation,
wherein the mutually exclusive orthogonal pair of reactive residues are selected from:
one of (S)-2-amino-3-azido-propionic acid or (S)-2-amino-4-azido-butanoic acid and one of (S)-2-aminopent-4-ynoic acid or (S)-2-aminohex-5-ynoic acid;
2-amino-3-mercaptopropanoic acid (L-Cysteine) and one of (S)-2-aminobut-3-enoic acid or (S)-2-aminopro-4-enoic acid;
(S)-2-amino-2-((1R, 8S, 9r)-bicyclo[6.1.0]non-4-yn-9-yl) acetic acid and one of (S)-2-amino-3-azido-propionic acid or (S) 2-amino-4-azido-butanoic acid;
(S)-2-amino-3-(4-(6-methyl-1,2,4,5-tetrazin-3-yl)phenyl) propanoic acid and one of (S)-2-amino-3-cyanopropanoic acid or (S)-2-amino-4-cyanobutanoic acid; and
(S)-2-(N-tert-Butoxycarbonyl)-4-[N-[2-chlorobenzyloxycarbonyl]-N-methoxy]diaminobutanoic acid and (S)-2-amino-3-(3-formylphenyl) propanoic acid,
and wherein the number of the two or more different protomers multiplied by (n+2) is 24.

153. (canceled)

154. (canceled)

155. A method of preparing a multivalent vaccine, comprising:
preparing an asymmetrical circular tandem repeat protein (cTRP) according to claim 152, wherein proteins of two or more different strains or subtypes of a virus or proteins of two or more different viruses are each operably linked to R¹ or R² of a protomer;
wherein if the virus comprises an influenza, each protein of the influenza is fused to a trimerization domain, and the method further comprises inducing trimerization of each protein of the influenza, followed by conjugating the trimerized proteins of the influenza to R¹ or R² of a different protomer, before mixing the two or more different protomers to form the asymmetrical cTRP, and optionally each protein of the influenza is further fused with a sortase recognition motif LPXTG (SEQ ID NO:44) at the C-terminus, and R1 comprises a sortase recognition motif GGG, and the method further comprises adding sortase to a mixture containing the trimerized fusion proteins of the influenza and the protomers, thereby operably linking or conjugating the trimerized fusion proteins of the influenza to the protomers.

156. (canceled)


SEQUENCE LISTING (553 sequences)

SEQ ID NO: 1 - protein, 205 aa, synthetic construct
GGGSCEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKCGISSEEILELLRAAHELGLGGGS

SEQ ID NO: 2 - protein, 205 aa, synthetic construct
GGGSPEAIKAAAELGCAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAACLGKAGISSEEILELLRAAHELGLGGGS

SEQ ID NO: 3 - protein, 205 aa, synthetic construct
GGGSPEAIKAACELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSECILELLRAAHELGLGGGS

SEQ ID NO: 4 - protein, 205 aa, synthetic construct
GGGSPEAIKAAAELGKAGISCEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPECIKAAAELGKAGISSEEILELLRAAHELGLGGGS

SEQ ID NO: 5 - protein, 4 aa, synthetic construct
GGGS

SEQ ID NO: 6 - protein, 33 aa, synthetic construct
CEAIKAAAELGKAGISSEEILELLRAAHELGLD

SEQ ID NO: 7 - protein, 33 aa, synthetic construct
PEAIKAAAELGKAGISSEEILELLRAAHELGLD

SEQ ID NO: 8 - protein, 32 aa, synthetic construct
PEAIKAAAELGKCGISSEEILELLRAAHELGL

SEQ ID NO: 9 - protein, 33 aa, Synthetic construct
PEAIKAAAELGCAGISSEEILELLRAAHELGLD

SEQ ID NO: 10 - protein, 32 aa, synthetic construct
PEAIKAAACLGKAGISSEEILELLRAAHELGL

SEQ ID NO: 11 - protein, 33 aa, synthetic construct
PEAIKAACELGKAGISSEEILELLRAAHELGLD

SEQ ID NO: 12 - protein, 32 aa, synthetic construct
PEAIKAAAELGKAGISSECILELLRAAHELGL

SEQ ID NO: 13 - protein, 33 aa, synthetic construct
PEAIKAAAELGKAGISCEEILELLRAAHELGLD

SEQ ID NO: 14 - protein, 32 aa, synthetic construct
PECIKAAAELGKAGISSEEILELLRAAHELGL

SEQ ID NO: 15 - protein, 13 aa, synthetic construct
PEAIKAAAELGKA

SEQ ID NO: 16 - protein, 14 aa, synthetic construct
SEEILELLRAAHEL

SEQ ID NO: 17 - protein, 11 aa, synthetic construct
GGSGGGSGGSG

SEQ ID NO: 18 - protein, 10 aa, synthetic construct
GGSGGGSGSG

SEQ ID NO: 19 - protein, 8 aa, synthetic construct
GGSGGGSG

SEQ ID NO: 20 - protein, 10 aa, synthetic construct
GGGGSGGGGS

SEQ ID NO: 21 - protein, 8 aa, synthetic construct
GGGSGGGS

SEQ ID NO: 22 - protein, 6 aa, synthetic construct
GGSGGS

SEQ ID NO: 23 - protein, 443 aa, synthetic construct
MRTVLLPWASAAVFLLVTVTVSGDEALEGVLQLQMINFNFEAVQVTWNASQYSGTNLTFLYKLTSDETDSQCSNYILQQSRTAGCLLEAKEDEILCFSIWNGTRLLLNKCQWISAYLKPSSPKDLNFQWHQEAITVTCSDLPYKRLLYEIQYKSMFDTEWQSKEGETCNVTTDGLDAEKCYFFRARVKTMESSYGPDTYPSDWSEVIHQQKGELRDSCQEKKLFPKGGGGSGGGGSCEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKCGISSEEILELLRAAHELGLGGGSHHHHHH

SEQ ID NO: 24 - protein, 443 aa, synthetic construct
MAWALAVILLPRLLAAAAAAAAVTSRGDVTVVCHDLETVEVTWGSGPDHHGANLSLEFRYGTGALQPCPRYFLSGAGVTSGCILPAARAGLLELALRDGGGAMVFKARQRASAWLKPRPPWNVTLLWTPDGDVTVSWPAHSYLGLDYEVQHRESNDDEDAWQTTSGPCCDLTVGGLDPARCYDFRVRASPRAAHYGLEAQPSEWTAVTRLSGAASAASCTASPAPSPALAPPGGGGSGGGGSPEAIKAAAELGCAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAACLGKAGISSEEILELLRAAHELGLGGGS

SEQ ID NO: 25 - protein, 449 aa, synthetic construct
MRAVTWAIVAMLLPRVLGAIPTRTPRTGGVGDTLSVAIVCHDLESVEVTWGPGSAHHGLSANLSLEFRYGNQVPQPCPHYFLLDSVRAGCVLPMGKGLLEVVLREGGGAKLFSRKKKASAWLRPRPPWNVTLSWVGDTVAVSCPSHSYPGLEYEVQHRDDFDPEWQSTSAPFCNLTVGGLDPGRCYDFRVRATPQDFYYGPEARPSKWTGVASLQGVGPTGSCTGPTLPRTPGTPTPPGGGGSGGGGSPEAIKAAAELGCAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAACLGKAGISSEEILELLRAAHELGLGGGS

SEQ ID NO: 26 - protein, 447 aa, synthetic construct
MGRLVLLWGAAVFLLGSWMALGQVATGEGLQIQIMYFNLETVQVTWNASHYPRSNLSFHYKFSRDEAYDQCTVYILQEGHTSGCLLDAQQQDDILYFSIRNGTHPVFTASRWIFYYLKPSSPKQVSFSWHQDAVTLTCSDLSYRGLLYEVQYRSPFDTEWQSKQENTCNVTIEDLDAEKCYAFRARVKAMEDAYGPDTYPSDWSEVTCWQRGKTRDSCPEPRTPPKPKLSKGGGGSGGGGSPEAIKAACELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSECILELLRAAHELGLGGGSDYKDE

SEQ ID NO: 27 - protein, 452 aa, synthetic construct
MGRLVLLWGAAVFLLGGWMALGQGGAAEGVQIQIIYFNLETVQVTWNASKYSRTNLTFHYRFNGDEAYDQCTNYLLQEGHTSGCLLDAEQRDDILYFSIRNGTHPVFTASRWMVYYLKPSSPKHVRFSWHQDAVTVTCSDLSYGDLLYEVQYRSPFDTEWQSKQENTCNVTIEGLDAEKCYSFWVRVKAMEDVYGPDTYPSDWSEVTCWQRGEIRDACAETPTPPKPKLSKGGGGSGGGGSPEAIKAAAELGKAGISCEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPECIKAAAELGKAGISSEEILELLRAAHELGLGGGSSAWSHPQFEK

SEQ ID NO: 28 - protein, 351 aa, synthetic construct
MRMFSVFTFMAYCHLLKAFTITVSKDLYVVEYGGNVTMECKFPVEKQLNLFALIVYWEMEDKKIIQFVNGKEDLKVQHSSYSQRAQLLKDQLFLGKAALQITDVRLQDAGVYCCLIGYGGADYKRITLKVHAPYGGGGSGGGGSCEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKCGISSEEILELLRAAHELGLGGGSHHHHHH

SEQ ID NO: 29 - protein, 345 aa, synthetic construct
MRIFAGIIFTACCHLLRAFTITAPKDLYVVEYGSNVTMECRFPVERELDLLALVVYWEKEDEQVIQFVAGEEDLKPQHSNFRGRASLPKDQLLKGNAALQITDVKLQDAGVYCCIISYGGADYKRITLKVNAPYGGGGSGGGGSPEAIKAAAELGCAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAACLGKAGISSEEILELLRAAHELGLGGGS

SEQ ID NO: 30 - protein, 345 aa, synthetic construct
MRIFAVLIVTACSHVLAAFTITAPKDLYVVEYGSNVTMECRFPVEQKLDLLALVVYWEKEDKEVIQFVEGEEDLKPQHSSFRGRAFLPKDQLLKGNAVLQITDVKLQDAGVYCCMISYGGADYKRITLKVNAPYGGGGSGGGGSPEAIKAAAELGCAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAACLGKAGISSEEILELLRAAHELGLGGGS

SEQ ID NO: 31 - protein, 350 aa, synthetic construct
MRIFAVFIFTIYWHLLNAFTVTVPKDLYVVEYGSNMTIECKFPVEKQLDLTSLIVYWEMEDKNIIQFVHGEEDLKVQHSNYRQRAQLLKDQLSLGNAALRITDVKLQDAGVYRCMISYGGADYKRITVKVNAPYGGGGSGGGGSPEAIKAACELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSECILELLRAAHELGLGGGSDYKDE

SEQ ID NO: 32 - protein, 355 aa, synthetic construct
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYGGGGSGGGGSPEAIKAAAELGKAGISCEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPECIKAAAELGKAGISSEEILELLRAAHELGLGGGSSAWSHPQFEK

SEQ ID NO: 33 - protein, 241 aa, synthetic construct
METDTLLLWVLLLWVPGSTGDYKDERRRKRGGGGSCEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKCGISSEEILELLRAAHELGLGGSHHHHHH

SEQ ID NO: 34 - protein, 235 aa, synthetic construct
METDTLLLWVLLLWVPGSTGDYKDERRRKRGGGGSPEAIKAAAELGCAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHECGLGGS

SEQ ID NO: 35 - protein, 240 aa, synthetic construct
METDTLLLWVLLLWVPGSTGDYKDERRRKRGGGGSPEAIKAAAELGKAGISSEEILELLRAACELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSECILELLRAAHELGLGGSDYKDE

SEQ ID NO: 36 - protein, 244 aa, synthetic construct
METDTLLLWVLLLWVPGSTGDYKDERRRKRGGGGSPEAIKAAAELGKAGISCEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPECIKAAAELGKAGISSEEILELLRAAHELGLGGSAWSHPQFEK

SEQ ID NO: 37 - protein, 20 aa, synthetic construct
METDTLLLWVLLLWVPGSTG

SEQ ID NO: 38 - protein, 5 aa, synthetic construct
DYKDE

SEQ ID NO: 39 - protein, 5 aa, synthetic construct
RRRKR

SEQ ID NO: 40 - protein, 6 aa, synthetic construct
HHHHHH

SEQ ID NO: 41 - protein, 9 aa, synthetic construct
AWSHPQFEK

SEQ ID NO: 42 - protein, 27 aa, synthetic construct
GYIPEAPRDGQAYVRKDGEWVLLSTFL

SEQ ID NO: 43 - protein, 5 aa, synthetic construct
LPETG

SEQ ID NO: 44 - protein, 5 aa, synthetic construct
  VARIANT 3: note = X can be any amino acid
LPXTG

SEQ ID NO: 45 - protein, 7 aa, synthetic construct
  VARIANT 3: note = X can be any amino acid
LPXTGGG

SEQ ID NO: 46 - protein, 572 aa, synthetic construct
METDTLLLWVLLLWVPGSTGDYKDEDTLCIGYHANNSTDTVDTVLEKNVTVTHSVNLLEDKHNGKLCKLRGVAPLHLGKCNIAGWILGNPECESLSTASSWSYIVETSNSDNGTCFPGDFINYEELREQLSSVSSFERFEIFPKTSSWPNHDSNKGVTAACPHAGAKSFYKNLIWLVKKGNSYPKLNQSYINDKGKEVLVLWGIHHPSTTADQQSLYQNADAYVFVGTSRYSKKFKPEIATRPKVRDQEGRMNYYWTLVEPGDKITFEATGNLVVPRYAFTMERNAGSGIIISDTPVHDCNTTCQTPEGAINTSLPFQNIHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVGSGYIPEAPRDGQAYVRKDGEWVLLSTFLGSGGLPETGHHHHHH

SEQ ID NO: 47 - protein, 574 aa, synthetic construct
METDTLLLWVLLLWVPGSTGDYKDEQKIPGNDNSTATLCLGHHAVPNGTIVKTITNDRIEVTNATELVQNSSIGEICDSPHQILDGENCTLIDALLGDPQCDGFQNKKWDLFVERSKAYSNCFPYDVPDYASLRSLVASSGTLEFNNESFNWTGVTQNGTSSACIRRSSSSFFSRLNWLTHLNYTYPALNVTMPNNEQFDKLYIWGVHHPGTDKDQIFLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKHSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHNVYRDEALNNRFQIKGVGSGYIPEAPRDGQAYVRKDGEWVLLSTFLGSGGLPETGHHHHHH

SEQ ID NO: 48 - protein, 592 aa, synthetic construct
METDTLLLWVLLLWVPGSTGDYKDEDRICTGITSSNSPHVVKTATQGEVNVTGVIPLTTTPTKSYFANLKGTRTRGKLCPDCLNCTDLDVALGRPMCVGTTPSAKASILHEVRPVTSGCFPIMHDRTKIRQLPNLLRGYEKIRLSTQNVIDAEKAPGGPYRLGTSGSCPNATSKIGFFATMAWAVPKDNYKNATNPLTVEVPYICTEGEDQITVWGFHSDNKTQMKSLYGDSNPQKFTSSANGVTTHYVSQIGDFPDQTEDGGLPQSGRIVVDYMMQKPGKTGTIVYQRGVLLPQKVWCASGRSKVIKGSLPLIGEADCLHEEYGGLNKSKPYYTGKHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLGSGYIPEAPRDGQAYVRKDGEWVLLSTFLGSGGLPETGHHHHHH

SEQ ID NO: 49 - protein, 593 aa, synthetic construct
METDTLLLWVLLLWVPGSTGDYKDEDRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHIRLSTHNVINAENAPGGPYKIGTSGSCPNITNGNGFFATMAWAVPKNDKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLGSGYIPEAPRDGQAYVRKDGEWVLLSTFLGSGGLPETGHHHHHH

SEQ ID NO: 50 - protein, 207 aa, Homo sapiens
GAAEGVQIQIIYFNLETVQVTWNASKYSRTNLTFHYRFNGDEAYDQCTNYLLQEGHTSGCLLDAEQRDDILYFSIRNGTHPVFTASRWMVYYLKPSSPKHVRFSWHQDAVTVTCSDLSYGDLLYEVQYRSPFDTEWQSKQENTCNVTIEGLDAEKCYSFWVRVKAMEDVYGPDTYPSDWSEVTCWQRGEIRDACAETPTPPKPKLSK

SEQ ID NO: 51 - protein, 209 aa, Macaca fascicularis
QVATGEGLQIQIMYFNLETVQVTWNASHYPRSNLSFHYKFSRDEAYDQCTVYILQEGHTSGCLLDAQQQDDILYFSIRNGTHPVFTASRWIFYYLKPSSPKQVSFSWHQDAVTLTCSDLSYRGLLYEVQYRSPFDTEWQSKQENTCNVTIEDLDAEKCYAFRARVKAMEDAYGPDTYPSDWSEVTCWQRGKTRDSCPEPRTPPKPKLSK

SEQ ID NO: 52 - protein, 203 aa, Canis lupus
DEALEGVLQLQMINFNFEAVQVTWNASQYSGTNLTFLYKLTSDETDSQCSNYILQQSRTAGCLLEAKEDEILCFSIWNGTRLLLNKCQWISAYLKPSSPKDLNFQWHQEAITVTCSDLPYKRLLYEIQYKSMFDTEWQSKEGETCNVTTDGLDAEKCYFFRARVKTMESSYGPDTYPSDWSEVIHQQKGELRDSCQEKKLFPK

SEQ ID NO: 53 - protein, 212 aa, Mus musculus
AAVTSRGDVTVVCHDLETVEVTWGSGPDHHGANLSLEFRYGTGALQPCPRYFLSGAGVTSGCILPAARAGLLELALRDGGGAMVFKARQRASAWLKPRPPWNVTLLWTPDGDVTVSWPAHSYLGLDYEVQHRESNDDEDAWQTTSGPCCDLTVGGLDPARCYDFRVRASPRAAHYGLEAQPSEWTAVTRLSGAASAASCTASPAPSPALAPP

SEQ ID NO: 54 - protein, 33 aa, synthetic construct
AEALKAAAKLAKAGVSAEEIAKILKEAKKLGLD

SEQ ID NO: 55 - protein, 33 aa, synthetic construct
EEYLKAAAELAKAGVSAEDIAKLLKLAKELGLK

SEQ ID NO: 56 - protein, 33 aa, synthetic construct
AEELEAAAKLAKAGVSAEEIKKLLKEAKKLGLK

SEQ ID NO: 57 - protein, 33 aa, synthetic construct
AEALKAAAELAKAGVSAEEIKKILKEAKKLGLG

SEQ ID NO: 58 - protein, 33 aa, synthetic construct
AEELKAAAELAKAGVSAEEIKKILEKAKELGLD

SEQ ID NO: 59 - protein, 33 aa, synthetic construct
SEALKAAAELAKAGVSAEEIKKILKEAKKLGLG

SEQ ID NO: 60 - protein, 33 aa, synthetic construct
EEALKAAAELAKAGVSAEEVKKLLKEAKKLGLG

SEQ ID NO: 61 - protein, 33 aa, synthetic construct
AEELKAAAKLAKAGVSAEEIKEILKEAKKLGLG

SEQ ID NO: 62 - protein, 33 aa, synthetic construct
AEELKAAAKLAKAGVSAEDIKKILKEAKELGLS

SEQ ID NO: 63 - protein, 33 aa, synthetic construct
AEALKAAAELAKAGVSAEEIKKLLKEAKQLGLG

SEQ ID NO: 64 - protein, 33 aa, synthetic construct
AKELEAAAKLAKAGVSAEEIKKILKEAKKLGLD

SEQ ID NO: 65 - protein, 33 aa, synthetic construct
AEALKAAAKLAKAGVSAEEIKKILKEAKKLGLG

SEQ ID NO: 66 - protein, 33 aa, synthetic construct
AEELKAAAKLAKAGVSAEEIKKILREAKELGLS

SEQ ID NO: 67 - protein, 33 aa, synthetic construct
AEALKAAAELAKAGVSAEEIKKLLKEAKKLGLG

SEQ ID NO: 68 - protein, 33 aa, synthetic construct
AEALKAAAELAKAGVSAEEIAKLLKEAAKLGLE

SEQ ID NO: 69 - protein, 33 aa, synthetic construct
AEALKAAAELAKAGVSAEEIKEILKEAKKLGLD

SEQ ID NO: 70 - protein, 33 aa, synthetic construct
EEALKAAAELAKAGVSAEEIKEILKEAKELGLG

SEQ ID NO: 71 - protein, 33 aa, synthetic construct
AEALKAAAELAKAGVSAEDILRLLREAARLGLG

SEQ ID NO: 72 - protein, 33 aa, synthetic construct
AEALEAAAKLAKAGVSAEEIKKILKEAKELGLG

SEQ ID NO: 73 - protein, 33 aa, synthetic construct
AEEIAAAAKLAEAGVSAEEIKAILKEAKALGLS

SEQ ID NO: 74 - protein, 33 aa, synthetic construct
AEALKAAAKLAKAGVSAEEIKELLKEAKKLGLG

SEQ ID NO: 75 - protein, 33 aa, synthetic construct
AEALKAAAELAKAGVSAEEIAKLLKEAKALGLD

SEQ ID NO: 76 - protein, 33 aa, synthetic construct
AKELEAAAKLAKAGVSAKEIKELLKEAKKLGLE

SEQ ID NO: 77 - protein, 33 aa, synthetic construct
AEELKAAAKLAKAGVSAEEIKKILKEAKKLGLG

SEQ ID NO: 78 - protein, 33 aa, synthetic construct
AEELKAAAELAKAGVSAEEIKELLKEAKKLGLD

SEQ ID NO: 79 - protein, 33 aa, synthetic construct
AEALKAAAELAKAGVSAEEIKKILKEAKKLGLP

SEQ ID NO: 80 - protein, 33 aa, synthetic construct
AEELKAAAELAKAGVSAEEIAKLLREAKELGLG

SEQ ID NO: 81 - protein, 33 aa, synthetic construct
AEELKAAAELAKAGVSAEEVKKLLREAKELGLG

SEQ ID NO: 82 - protein, 33 aa, synthetic construct
AEALKAAAELAKAGVSAEEIAKLLELAAKLGLD

SEQ ID NO: 83 - protein, 33 aa, synthetic construct
AEWLKAAAELAKAGVSAKEIKELLKEAKKLGLD

SEQ ID NO: 84 - protein, 33 aa, synthetic construct
AEELEAAAKLAKAGVSAEEIKELLEEAKRLGLG

SEQ ID NO: 85 - protein, 33 aa, synthetic construct
AEALKAAAELAKAGVSAEEIEKLLKEAKKLGLG

SEQ ID NO: 86 - protein, 33 aa, synthetic construct
AEALKAAAKLAKAGISAEEIKKILKEAKKLGLS

SEQ ID NO: 87 - protein, 33 aa, synthetic construct; identical to SEQ ID NO: 57

SEQ ID NO: 88 - protein, 33 aa, synthetic construct
AEALKAAAKLAEAGVSAEEIKKILKEAKKLGLS

SEQ ID NO: 89 - protein, 33 aa, synthetic construct
EEAIKAAAELAKAGVSAEEIKKILKEAKKLGLG

SEQ ID NO: 90 - protein, 33 aa, synthetic construct
AEALKAAAELAKAGVSAEEIKKILKEAKELGLG

SEQ ID NO: 91 - protein, 33 aa, synthetic construct
AEALKAAAKLAEAGVSAEDVAKLLREAKELGLP

SEQ ID NO: 92 - protein, 33 aa, synthetic construct
AEELKAAAELAKAGVSAEEIKKLLREAKALGLS

SEQ ID NO: 93 - protein, 33 aa, synthetic construct
AEALKAAAKLAKAGVSAEEIKRILREAKRLGLG

SEQ ID NO: 94 - protein, 33 aa, synthetic construct
AEELKAAAELAKAGVSAKEIKELLKEAKKLGLG

SEQ ID NO: 95 - protein, 33 aa, synthetic construct; identical to SEQ ID NO: 60

SEQ ID NO: 96 - protein, 33 aa, synthetic construct
AEALKAAAKLAKAGVSAEEVKKLLKEAKKLGLG

SEQ ID NO: 97 - protein, 33 aa, synthetic construct
AEALKAAAELAKAGVSAEEIAALLKEAKALGLG

SEQ ID NO: 98 - protein, 33 aa, synthetic construct; identical to SEQ ID NO: 57

SEQ ID NO: 99 - protein, 33 aa, synthetic construct
AEALKAAAELAKAGVSAEEIKKILKEAKELGLS

SEQ ID NO: 100 - protein, 33 aa, synthetic construct
SEALKAAAKLAEAGVSAEEIEKLLKLAKELGLG

SEQ ID NO: 101 - protein, 33 aa, synthetic construct
AEALKAAAKLAKAGVSAEEIKKLLEEAKKLGLG

SEQ ID NO: 102 - protein, 33 aa, synthetic construct
AEALKAAAELAKAGVSAEEIKELLREAKELGLD

SEQ ID NO: 103 - protein, 33 aa, synthetic construct
AEALKAAAELAKAGVSAEEIKKILKEAKELGLE

SEQ ID NO: 104 - protein, 33 aa, synthetic construct
AEALKAAAKLAKAGVSAEEIKKLLKEAKKLGLG

SEQ ID NO: 105 - protein, 33 aa, synthetic construct
SEALKAAAKLAKAGVSAEEIKKILKEAKKLGLS

SEQ ID NO: 106 - protein, 33 aa, synthetic construct
AEELKAAAELAKAGVSAEEVMKILEKAKELGLD

SEQ ID NO: 107 - protein, 33 aa, synthetic construct
SEALKAAAKLAEAGVSAEEVAKLLREAAELGLG

SEQ ID NO: 108 - protein, 33 aa, synthetic construct
AEALKAAAELAKAGVSAEEILKILKEAKKLGLE

SEQ ID NO: 109 - protein, 33 aa, synthetic construct
AEAIKAAAELAKAGVSAEEIKKILKEAKELGLG

SEQ ID NO: 110 - protein, 33 aa, synthetic construct
AEELKAAAELAKAGISAEEIKKLLKEAKKLGLK

SEQ ID NO: 111 - protein, 33 aa, synthetic construct
AETLKAAAELAKAGVSAEEIKKILKEAKKLGLG

SEQ ID NO: 112 - protein, 33 aa, synthetic construct; identical to SEQ ID NO: 67

SEQ ID NO: 113 - protein, 33 aa, synthetic construct
AEALKAAAKLAKAGVSAEEIKEILKEAKKLGLG

SEQ ID NO: 114 - protein, 33 aa, synthetic construct
AEELKAAAELAKAGISAEEIKKLLKEAKKLGLG

SEQ ID NO: 115 - protein, 33 aa, synthetic construct
AEELKAAAKLAEAGVSAEDIAKILKEAAKLGLK

SEQ ID NO: 116 - protein, 33 aa, synthetic construct
AEAIKAAAELAKAGVSAEEIIKILKEAKKLGLG

SEQ ID NO: 117 - protein, 33 aa, synthetic construct
AESLAAAAKLAKAGVSAEEIAKLLKEAKELGLD

SEQ ID NO: 118 - protein, 33 aa, synthetic construct
AEAIKAAAELAKAGISAEEIEKILKEAKKLGLG

SEQ ID NO: 119 - protein, 33 aa, synthetic construct
AEALKAAAELAKAGVSAEEIAKLLKEAKKLGLG

SEQ ID NO: 120 - protein, 33 aa, synthetic construct
AKALEAAAKLAKAGVSAEEIMKILKEAKKLGLD

SEQ ID NO: 121 - protein, 33 aa, synthetic construct
AEELKAAAELAKAGVSAEDVKKLLKEAKKLGLG

SEQ ID NO: 122 - protein, 33 aa, synthetic construct; identical to SEQ ID NO: 113

SEQ ID NO: 123 - protein, 33 aa, synthetic construct
AEELKAAAELAKAGVSAKEIKELLKEAKKLGLK

SEQ ID NO: 124 - protein, 33 aa, synthetic construct
AEELKAAAELAKAGISAEEIKKILKEAKKLGLG

SEQ ID NO: 125 - protein, 33 aa, synthetic construct
AEALKAAAKLAKAGVSAEEIKEILKLAKELGLG

SEQ ID NO: 126 - protein, 33 aa, synthetic construct
AEALAAAAKLAKAGVSAEEIKKLLKEAKKLGLD

SEQ ID NO: 127 - protein, 33 aa, synthetic construct
SKAIKAAAELAKAGVSAEEIKKILKEAKKLGLE

SEQ ID NO: 128 - protein, 33 aa, synthetic construct
AEELKAAAKLAKAGVSAEEVKEILKLAKELGLK

SEQ ID NO: 129 - protein, 33 aa, synthetic construct
AETLKAAAELAKAGVSAEEIKKIMKEAKKLGLD

SEQ ID NO: 130 - protein, 33 aa, synthetic construct; identical to SEQ ID NO: 69

SEQ ID NO: 131 - protein, 33 aa, synthetic construct
EEALKAAAELAKAGVSAEEIKKILKEAKKLGLK

SEQ ID NO: 132 - protein, 33 aa, synthetic construct
AEWLKAAAKLAKAGVSAEEIKKILKEAKKLGLE

SEQ ID NO: 133 - protein, 33 aa, synthetic construct
AEALKAAAELAKAGVSAEEIKELLKEAKKLGLG

SEQ ID NO: 134 - protein, 33 aa, synthetic construct
AEELKAAAELAKAGVSAEEIKKILKEAKALGLG

SEQ ID NO: 135 - protein, 33 aa, synthetic construct
AEELKAAAELAKAGISAEEIEKLLKTAKELGLD

SEQ ID NO: 136 - protein, 33 aa, synthetic construct
AEELKAAAELAKAGVSAEEIKKILKEAKKLGLK

SEQ ID NO: 137 - protein, 33 aa, synthetic construct
AESLKAAAELAKAGVSAKEIKELLKEAKKLGLD

SEQ ID NO: 138 - protein, 33 aa, synthetic construct
AEALKAAAELAKAGVSAKEIKEILKEAKKLGLG

SEQ ID NO: 139 - protein, 33 aa, synthetic construct
AESLKAAAELAKAGVSAEEVKELLELAKKLGLG

SEQ ID NO: 140 - protein, 33 aa, synthetic construct
AEALKAAAELAKAGVSAEDIAKILKEAAKLGLD

SEQ ID NO: 141 - protein, 33 aa, synthetic construct
EEFLKAAAKLAKAGVSAEEIKKLLKEAKKLGLK

SEQ ID NO: 142 - protein, 33 aa, synthetic construct; identical to SEQ ID NO: 57

SEQ ID NO: 143 - protein, 33 aa, synthetic construct; identical to SEQ ID NO: 69

SEQ ID NO: 144 - protein, 33 aa, synthetic construct
AESLKAAAELAKAGVSAEEIKEILKLAKELGLD

SEQ ID NO: 145 - protein, 33 aa, synthetic construct
AEALKAAAELAKAGVSAEEIKKLLKEAKKLGLD

SEQ ID NO: 146 - protein, 33 aa, synthetic construct
AEYLKAAAELAKAGVSAEEILKILKEAKKLGLK

SEQ ID NO: 147 - protein, 33 aa, synthetic construct
EEALKAAAELAKAGVSAEEIAKLLKEAKKLGLK

SEQ ID NO: 148 - protein, 33 aa, synthetic construct
AEELKAAAELAKAGVSAEEVKKLLKEAKKLGLG

SEQ ID NO: 149 - protein, 33 aa, synthetic construct
AEELKAAAKLAKAGVSAEDILKILKEAKKLGLK

SEQ ID NO: 150 - protein, 33 aa, synthetic construct; identical to SEQ ID NO: 57

SEQ ID NO: 151 - protein, 33 aa, synthetic construct
AKELEAAAKLAEAGVSAEEIERILREAKELGLD

SEQ ID NO: 152 - protein, 33 aa, synthetic construct
AEALKAAAELAKAGISAEEIAKLLREAKELGLG

SEQ ID NO: 153 - protein, 33 aa, synthetic construct
AEELKAAAELAKAGVSAEEIKKLLKEAKKLGLG

SEQ ID NO: 154 - protein, 33 aa, synthetic construct
AKALEAAAKLAKAGVSAEEIKKLLELAKKLGLD

SEQ ID NO: 155 - protein, 33 aa, synthetic construct; identical to SEQ ID NO: 67

SEQ ID NO: 156 - protein, 33 aa, synthetic construct
AEALKAAAELAKAGISAEEIKKLLKEAKKLGLD

SEQ ID NO: 157 - protein, 33 aa, synthetic construct; identical to SEQ ID NO: 153

SEQ ID NO: 158 - protein, 33 aa, synthetic construct
AEALKAAAELAKAGVSAEEIMALLREAKELGLG

SEQ ID NO: 159 - protein, 33 aa, synthetic construct
AEELKAAAELAKAGVSAEEIKKLLKEAKKLGLK

SEQ ID NO: 160 - protein, 33 aa, Synthetic construct
EEYLKAAAKLAKAGVSAEEIKKILKEAKELGLD

SEQ ID NO: 161 - protein, 33 aa, synthetic construct; identical to SEQ ID NO: 104

SEQ ID NO: 162 - protein, 33 aa, synthetic construct
AEALKAAAKLAKAGVSAEEIKKILKEAKELGLD

SEQ ID NO: 163 - protein, 33 aa, synthetic construct
AEALKAAAELAKAGVSAEDIKKILEEAKKLGLG

SEQ ID NO: 164 - protein, 33 aa, synthetic construct
AKAIEAAAKLAKAGVSAEEIKEILKEAAKLGLD

SEQ ID NO: 165 - protein, 33 aa, synthetic construct
AEALKAAAELAKAGVSAEEIKALLKEAAALGLG

SEQ ID NO: 166 - protein, 33 aa, synthetic construct; identical to SEQ ID NO: 65

SEQ ID NO: 167 - protein, 33 aa, synthetic construct
AEELKAAAELAKAGVSAEEIAKLLKEAKALGLG

SEQ ID NO: 168 - protein, 33 aa, synthetic construct
AEALKAAAELAKAGVSAEEIKKILKEAKKLGLD

SEQ ID NO: 169 - protein, 33 aa, synthetic construct; identical to SEQ ID NO: 67

SEQ ID NO: 170 - protein, 33 aa, synthetic construct
AEEIKAAAELAKAGVSAEEIKKILKEAKELGLE

SEQ ID NO: 171 - protein, 33 aa, synthetic construct
AEELKAAAKLAEAGVSAEEILRLLKEAKKLGLS

SEQ ID NO: 172 - protein, 33 aa, synthetic construct
AEAIKAAAELAKAGVSAKEIKEILKEAKKLGLG

SEQ ID NO: 173 - protein, 33 aa, synthetic construct
AEELEAAAKLAKAGVSAEEIKKILKEAKKLGLG

SEQ ID NO: 174 - protein, 33 aa, synthetic construct
AEELAAAAKLAKAGVSAEEVAKLLKLAKELGLG

SEQ ID NO: 175 - protein, 33 aa, synthetic construct; identical to SEQ ID NO: 74

SEQ ID NO: 176 - protein, 33 aa, synthetic construct
AEALKAAAELAKAGVSAEEIAKILKEAKKLGLG

SEQ ID NO: 177 - protein, 33 aa, synthetic construct
AEALKAAAELAKAGVSAKEIKELLKEAKKLGLD

SEQ ID NO: 178 - protein, 33 aa, synthetic construct
AEELKAAAELAKAGVSAKEIKELLELAKKLGLK

SEQ ID NO: 179 - protein, 33 aa, synthetic construct
AEALKAAAKLAEAGVSAEEIAKLLKEAKKLGLG

SEQ ID NO: 180 - protein, 33 aa, synthetic construct
AEALKAAAELAKAGVSAKEIKELLKEAKKLGLE

SEQ ID NO: 181 - protein, 33 aa, synthetic construct
AEALKAAAELAKAGVSAEEIKKLLKEAAKLGLG

SEQ ID NO: 182 - protein, 33 aa, synthetic construct
EEELKAAAELAKAGVSAKEIKELLKEAKKLGLK

SEQ ID NO: 183 - protein, 33 aa, synthetic construct
AEELKAAAELAKAGVSAEEIKKLLKEAKELGLG

SEQ ID NO: 184 - protein, 33 aa, synthetic construct
AEDLKAAAKLAKAGVSAEEIKKILKEAKKLGLK

SEQ ID NO: 185 - protein, 33 aa, synthetic construct
AEALKAAAELAKAGVSAEEIKALLKEAKKLGLG

SEQ ID NO: 186 - protein, 33 aa, synthetic construct
AKALEAAAKLAKAGVSAEEIAALLKQAKALGLD

SEQ ID NO: 187 - protein, 33 aa, synthetic construct
AEALKAAAELAKAGVSPEEIKKLLKEAKELGLG

SEQ ID NO: 188 - protein, 33 aa, synthetic construct; identical to SEQ ID NO: 57

SEQ ID NO: 189 - protein, 33 aa, synthetic construct; identical to SEQ ID NO: 77

SEQ ID NO: 190 - protein, 33 aa, synthetic construct
AEALKAAAELAKAGVSAEEIKKILKEAKELGLD

SEQ ID NO: 191 - protein, 33 aa, synthetic construct
AEALKAAAELAKAGVSAEEVKKILKEAKKLGLG

SEQ ID NO: 192 - protein, 33 aa, synthetic construct
AEELKAAAELAKAGVSAEEVKKILKEAKKLGLS

SEQ ID NO: 193 - protein, 33 aa, synthetic construct
AEAIKAAAELAKAGVSAEEIKKILKEAKKLGLD

SEQ ID NO: 194 - protein, 33 aa, synthetic construct
AEELKAAAELAKAGVSAEEIEKILKEAKKLGLK

SEQ ID NO: 195 - protein, 33 aa, synthetic construct
AEALKAAAKLAKAGVSAEEIKELLKLAKELGLG

SEQ ID NO: 196 - protein, 33 aa, synthetic construct
AEALKAAAELAKAGVSAKEVKELLKEAKKLGLG

SEQ ID NO: 197 - protein, 33 aa, synthetic construct; identical to SEQ ID NO: 109

SEQ ID NO: 198 - protein, 33 aa, synthetic construct
AKELEAAAKLAKAGVSAEEVAKLLKEAKKLGLG

SEQ ID NO: 199 - protein, 33 aa, synthetic construct
AEALKAAAELAKAGVSAEEVKELLKEAKKLGLS

SEQ ID NO: 200 - protein, 33 aa, synthetic construct; identical to SEQ ID NO: 136

SEQ ID NO: 201 - protein, 33 aa, synthetic construct
AEELKAAAELAKAGVSAKEIKEILKLAKELGLK

SEQ ID NO: 202 - protein, 33 aa, synthetic construct; identical to SEQ ID NO: 168

SEQ ID NO: 203 - protein, 33 aa, synthetic construct
AEALKAAAKLAKAGVSAEEIKKILKEAKKLGLK

SEQ ID NO: 204 - protein, 33 aa, synthetic construct
AEALKAAAELAKAGVSAEEIKEILEKAKELGLG

SEQ ID NO: 205 - protein, 33 aa, synthetic construct
AKALEAAAKLAKAGVSAKEIKEILKEAKKLGLS

SEQ ID NO: 206 - protein, 33 aa, synthetic construct
AEALKAAAKLAKAGISAEEIKKLLKEAKELGLG

SEQ ID NO: 207 - protein, 33 aa, synthetic construct
AEALKAAAELAKAGVSAEEIAALLEKAAALGLD

SEQ ID NO: 208 - protein, 33 aa, synthetic construct; identical to SEQ ID NO: 114

SEQ ID NO: 209 - protein, 33 aa, synthetic construct; identical to SEQ ID NO: 159

SEQ ID NO: 210 - protein, 33 aa, synthetic construct
AEALKAAAKLAKAGVSAEEIAKLLKEAKKLGLG

SEQ ID NO: 211 - protein, 33 aa, synthetic construct; identical to SEQ ID NO: 63

SEQ ID NO: 212 - protein, 33 aa, synthetic construct
AEALKAAAKLAKAGISAKEIKEILKEAKELGLG

SEQ ID NO: 213 - protein, 33 aa, synthetic construct
AEALKAAAKLAEAGVSAEEIKELLKEAKRLGLG

SEQ ID NO: 214 - protein, 33 aa, synthetic construct
AEALEAAAKLAKAGVSAEEIAKLLKLAKELGLD

SEQ ID NO: 215 - protein, 33 aa, synthetic construct
AEAIKAAAKLAKAGVSAEEIKKILKEAKKLGLS

SEQ ID NO: 216 - protein, 33 aa, synthetic construct
AEALKAAAELAKAGVSAKEIKEILKEAKELGLS

SEQ ID NO: 217 - protein, 33 aa, synthetic construct; identical to SEQ ID NO: 65

SEQ ID NO: 218 - protein, 33 aa, synthetic construct
AEYLKAAAELAKAGISAEEIKKLLEEAKKLGLK

SEQ ID NO: 219 - protein, 33 aa, synthetic construct
AEELKAAAELAKAGVSAEEIKKLLEEAKKLGLG

SEQ ID NO: 220 - protein, 33 aa, synthetic construct
SKSLEAAAKLAKAGVSAEEIKKILKEAKKLGLD

SEQ ID NO: 221 - protein, 33 aa, synthetic construct
AEALAAAAKLAKAGVSAEEIIKILREAKKLGLG

SEQ ID NO: 222 - protein, 33 aa, synthetic construct
AEAIKAAAELAKAGVSAKDIKAILKEAKALGLG

SEQ ID NO: 223 - protein, 33 aa, synthetic construct
AETLKAAAELAKAGVSAKEIKELLKEAKKLGLS

SEQ ID NO: 224 - protein, 33 aa, synthetic construct
AEELKAAAELAKAGVSAEEVAKLLREAKKLGLS

SEQ ID NO: 225 - protein, 33 aa, synthetic construct
AEALKAAAELAKAGVSAEEVKKLLKEAKKLGLD

SEQ ID NO: 226 - protein, 33 aa, synthetic construct
AEELKAAAELAKAGVSAEEILKILKEAKELGLD

SEQ ID NO: 227 - protein, 33 aa, synthetic construct
EEELKAAAELAKAGVSAEEIKKLLKEAKKLGLS

SEQ ID NO: 228 - protein, 33 aa, synthetic construct; identical to SEQ ID NO: 145

SEQ ID NO: 229 - protein, 33 aa, synthetic construct
AEEIKAAAELAKAGVSAKEIKEILKEAKKLGLK

SEQ ID NO: 230 - protein, 33 aa, synthetic construct
AEALKAAAELAKAGVSAEEVKKILKEAKALGLG

SEQ ID NO: 231 - protein, 33 aa, synthetic construct
AEALKAAAELAKAGVSAEDIAKLLKLAHELGLG

SEQ ID NO: 232 - protein, 33 aa, synthetic construct
AEALKAAAELAKAGVSAEDIKKLLKEAKELGLD

SEQ ID NO: 233 - protein, 33 aa, synthetic construct
AEELKAAAKLAKAGVSAEEIAKLLKEAKKLGLG

SEQ ID NO: 234 - protein, 33 aa, synthetic construct
AEALKAAAELAKAGVSAEEIQKLLKEAKALGLD

SEQ ID NO: 235 - protein, 33 aa, synthetic construct; identical to SEQ ID NO: 67

SEQ ID NO: 236 - protein, 33 aa, synthetic construct
AEALKAAAELAKAGVSAEEIKEILREAKELGLP

SEQ ID NO: 237 - protein, 33 aa, synthetic construct
AEALRAAAELAKAGVSAEEIAKLLREAKRLGLD

SEQ ID NO: 238 - protein, 33 aa, synthetic construct
AEALKAAAKLAKAGVSAEEIKELLKEAKELGLG

SEQ ID NO: 239 - protein, 33 aa, synthetic construct
AEELKAAAELAKAGVSAEDIAKLLKLAKELGLG

SEQ ID NO: 240 - protein, 33 aa, synthetic construct
AEALKAAAELAKAGVSAEEIEKILKEAAKLGLG

SEQ ID NO: 241 - protein, 33 aa, synthetic construct
EEFLKAAAELAKAGVSAEEIKKLLKEAKKLGLK

SEQ ID NO: 242 - protein, 33 aa, synthetic construct
AEALKAAAELAKAGVSAEEIKALLKEAAKLGLG

SEQ ID NO: 243 - protein, 33 aa, synthetic construct
AEALKAAAKLAKAGVSAEEIKKLLKEAKELGLD

SEQ ID NO: 244 - protein, 33 aa, synthetic construct
AEELEAAAKLAKAGVSAEEIKKILKEAAKLGLG

SEQ ID NO: 245 - protein, 33 aa, synthetic construct
AEALRAAAELAKAGVSAEEVERLLREAARLGLG

SEQ ID NO: 246 - protein, 33 aa, synthetic construct
AEELKAAAKLAEAGVSAEEVKKLLKEAKKLGLG

SEQ ID NO: 247 - protein, 33 aa, synthetic construct; identical to SEQ ID NO: 69

SEQ ID NO: 248 - protein, 33 aa, synthetic construct
AEALKAAAKLAKAGVSAEEVKAILKEAKALGLD

SEQ ID NO: 249 - protein, 33 aa, synthetic construct
AEELKAAAELAKAGVSAEEIAKLLEKAKELGLG

SEQ ID NO: 250 - protein, 33 aa, synthetic construct
AEELKAAAELAKAGVSAEEVKKILKEAKKLGLK

SEQ ID NO: 251 - protein, 33 aa, synthetic construct; identical to SEQ ID NO: 57

SEQ ID NO: 252 - protein, 33 aa, synthetic construct; identical to SEQ ID NO: 190

SEQ ID NO: 253 - protein, 33 aa, synthetic construct
AEELKAAAELAKAGVSAEDIKKILKEAKELGLE

SEQ ID NO: 254 - protein, 33 aa, synthetic construct
AEELKAAAELAKAGVSAEEIAKLLKEAKKLGLS

SEQ ID NO: 255 - protein, 33 aa, synthetic construct
AEELKAAAKLAKAGVSAEEIKKILKEAKELGLK

SEQ ID NO: 256 - protein, 33 aa, synthetic construct
AEALKAAAELAKAGVSPEEIKKILKEAKKLGLD

SEQ ID NO: 257 - protein, 33 aa, synthetic construct
AEELKAAAELAKAGVSAEEVEKLLREAKKLGLG

SEQ ID NO: 258 - protein, 33 aa, synthetic construct
EKALEAAAKLAKAGVSAKEIKEILKEAKELGLD

SEQ ID NO: 259 - protein, 33 aa, synthetic construct
AEALKAAAELAKAGVSAEEIKELLKKAKELGLG

SEQ ID NO: 260 - protein, 33 aa, synthetic construct
AEALKAAAELAKAGVSAEEIKELLKEAKELGLG

SEQ ID NO: 261 - protein, 33 aa, synthetic construct
AEALEAAAKLAKAGVSAEEIKKLLKEAKKLGLS

SEQ ID NO: 262 - protein, 33 aa, synthetic construct
AEELKAAAELAKAGVSAKDIAKILKEAKKLGLG

SEQ ID NO: 263 - protein, 33 aa, synthetic construct
AEALKAAAELAKAGVSAEEIKKLLKEAKELGLE

SEQ ID NO: 264 - protein, 33 aa, synthetic construct; identical to SEQ ID NO: 57

SEQ ID NO: 265 - protein, 33 aa, synthetic construct
AEYVKAAAKLAKAGVSAEEIKEILKEAKKLGLK

SEQ ID NO: 266 - protein, 33 aa, synthetic construct
AEALKAAAELAKAGISAEEIKELLKTAKALGLG

SEQ ID NO: 267 - protein, 33 aa, synthetic construct
AEALKAAAELAKAGVSAEDIKKLLKEAKKLGLE

SEQ ID NO: 268 - protein, 33 aa, synthetic construct
AEALKAAAELAKAGISAEEIKKILKEAKELGLD

SEQ ID NO: 269 - protein, 33 aa, synthetic construct; identical to SEQ ID NO: 159

SEQ ID NO: 270 - protein, 33 aa, synthetic construct
AEELKAAAELAKAGVSAEEILKLLKEAKKLGLG

SEQ ID NO: 271 - protein, 33 aa, synthetic construct
AEALKAAAELAKAGVSAEDIKKILKEAKKLGLG

SEQ ID NO: 272 - protein, 33 aa, synthetic construct
AEELKAAAELAKAGVSAEEIKKILKEAKKLGLD

SEQ ID NO: 273 - protein, 33 aa, synthetic construct
AEEIKAAAELAKAGVSAEEIKAILKEAKALGLE

SEQ ID NO: 274 - protein, 33 aa, synthetic construct
AEELKAAAKLAKAGVSAEEIKEILKEAKELGLK

SEQ ID NO: 275 - protein, 33 aa, synthetic construct
AEELKAAAKLAKAGISAKEIKEILKEAKKLGLG

SEQ ID NO: 276 - protein, 33 aa, synthetic construct
EEALKAAAELAKAGVSAEEIMKLLKEAKRLGLS

SEQ ID NO: 277 - protein, 33 aa, synthetic construct
AKALEAAAKLAKAGVSAEEIKEILKEAKKLGLD

SEQ ID NO: 278 - protein, 33 aa, synthetic construct
AEELAAAAELAKAGVSAEEVAALLKEAKALGLE

SEQ ID NO: 279 - protein, 33 aa, synthetic construct
AEALKAAAELAKAGVSAEEIAKLLREAKELGLS

SEQ ID NO: 280 - protein, 33 aa, synthetic construct
AEALKAAAKLAEAGVSAEEIKKLLKEAKKLGLD

SEQ ID NO: 281 - protein, 33 aa, synthetic construct; identical to SEQ ID NO: 90

SEQ ID NO: 282 - protein, 33 aa, synthetic construct
EEALKAAAELAKAGVSAEEIKKLLEEAKKLGLS

SEQ ID NO: 283 - protein, 33 aa, synthetic construct
SEALKAAAKLAKAGVSAEDIKKLLKEAKELGLE

SEQ ID NO: 284 - protein, 33 aa, synthetic construct; identical to SEQ ID NO: 57

SEQ ID NO: 285 - protein, 33 aa, synthetic construct
AESLKAAAKLAKAGVSAEEIKKILKEAKELGLS

SEQ ID NO: 286 - protein, 33 aa, synthetic construct
AEELKAAAELAKAGVSAEEIIKILKEAKRLGLG

SEQ ID NO: 287 - protein, 33 aa, synthetic construct
AEALKAAAKLAKAGVSAEEIKELLKEAKKLGLS

SEQ ID NO: 288 - protein, 33 aa, synthetic construct
AEALKAAAELAKAGVSAEEIAALLREAAALGLD

SEQ ID NO: 289 - protein, 33 aa, synthetic construct
AKALEAAAKLAKAGVSAEEIAKLLKEAKKLGLE

SEQ ID NO: 290 - protein, 33 aa, synthetic construct
AEAVKAAAKLAEAGVSAEEIKKILKEAKKLGLG

SEQ ID NO: 291 - protein, 33 aa, synthetic construct
AEAIKAAAKLAKAGVSAEEIKEILKEAKELGLE

SEQ ID NO: 292 - protein, 33 aa, synthetic construct
SEELKAAAELAKAGVSAEEIKEILEKAKKLGLK

SEQ ID NO: 293 - protein, 33 aa, synthetic construct; identical to SEQ ID NO: 145

SEQ ID NO: 294 - protein, 33 aa, synthetic construct
EEELKAAAELAKAGVSAEEIKKILKEAKELGLK

SEQ ID NO: 295 - protein, 33 aa, synthetic construct
AEALKAAAKLAKAGVSAEEIKKILKEAKELGLG

SEQ ID NO: 296 - protein, 33 aa, synthetic construct
EKELEAAAKLAKAGISAEEIKKILKEAKELGLG

SEQ ID NO: 297 - protein, 33 aa, synthetic construct
AEAIKAAAELAKAGVSAEEIKEILKEAKELGLG

SEQ ID NO: 298 - protein, 33 aa, synthetic construct
EKAIEAAAKLAKAGVSAEEIKKILEEAKKLGLS

SEQ ID NO: 299 - protein, 33 aa, synthetic construct
AEELKAAAELAKAGISAEEIKKILKEAKELGLS

SEQ ID NO: 300 - protein, 33 aa, synthetic construct
AEELKAAAELAKAGISAEEILKILEKAKKLGLG

SEQ ID NO: 301 - protein, 33 aa, synthetic construct
AEALAAAAKLAKAGVSAEEIEKILREAKELGLG

SEQ ID NO: 302 - protein, 33 aa, synthetic construct
AEELKAAAKLAKAGISAEEIKKILKEAKKLGLG

SEQ ID NO: 303 - protein, 33 aa, synthetic construct
AEALKAAAELAKAGVSAEEVKKLLREAKELGLD

SEQ ID NO: 304 - protein, 33 aa, synthetic construct
AEALKAAAKLAKAGVSAKEIKELLEKAKKLGLS

SEQ ID NO: 305 - protein, 33 aa, synthetic construct
AEALKAAAELAKAGVSAEEVKKLLKEAKKLGLG

SEQ ID NO: 306 - protein, 33 aa, synthetic construct; identical to SEQ ID NO: 67

SEQ ID NO: 307 - protein, 33 aa, synthetic construct
EEELKAAAKLAKAGVSAEEILKLLKEAKELGLK

SEQ ID NO: 308 - protein, 33 aa, synthetic construct; identical to SEQ ID NO: 133

SEQ ID NO: 309 - protein, 33 aa, synthetic construct
AEALKAAAELAKAGVSAEEIMKIMKEAKKLGLD

SEQ ID NO: 310 - protein, 33 aa, synthetic construct
AEELKAAAELAKAGVSAEEIKKILKEAKKLGLG

SEQ ID NO: 311 - protein, 33 aa, synthetic construct
AEALKAAAELAKAGVSAEEIKKLLKEAKKLGLS

SEQ ID NO: 312 - protein, 33 aa, synthetic construct; identical to SEQ ID NO: 65

SEQ ID NO: 313 - protein, 33 aa, synthetic construct
AEALKAAAELAKAGVSAEEIKELLKEAKKLGLD

SEQ ID NO: 314 - protein, 33 aa, synthetic construct
AEELKAAAKLAKAGVSAEEIKKLLKEAKKLGLG

SEQ ID NO: 315 - protein, 33 aa, synthetic construct
AEALKAAAELAKAGISAEEIKELLKEAKELGLD

SEQ ID NO: 316 - protein, 33 aa, synthetic construct
EEELKAAAELAKAGVSAEDVLKILREAKKLGLK

SEQ ID NO: 317 - protein, 33 aa, synthetic construct; identical to SEQ ID NO: 181

SEQ ID NO: 318 - protein, 33 aa, synthetic construct; identical to SEQ ID NO: 219

SEQ ID NO: 319 - protein, 33 aa, synthetic construct
AEALKAAAKLAKAGVSAKEIKELLKEAKKLGLG

SEQ ID NO: 320 - protein, 33 aa, synthetic construct; identical to SEQ ID NO: 57

SEQ ID NO: 321 - protein, 33 aa, synthetic construct
AEALKAAAELAKAGISAEEIKKILKEAKKLGLG

SEQ ID NO: 322 - protein, 33 aa, synthetic construct
AEALKAAAKLAKAGVSAEDIEKLLKEAKKLGLS

SEQ ID NO: 323 - protein, 33 aa, synthetic construct
AEALKAAAELAKAGVSAEEIKKLLKEAKALGLD

SEQ ID NO: 324 - protein, 33 aa, synthetic construct
AEELKAAAELAKAGISAEEIKEILKEAKKLGLG

SEQ ID NO: 325 - protein, 33 aa, synthetic construct
AEALKAAAELAKAGVSAEEVKALLKEAKELGLG

SEQ ID NO: 326 - protein, 33 aa, synthetic construct
AEALKAAAELAKAGVSAEEIKKILEKAKELGLD

SEQ ID NO: 327 - protein, 33 aa, synthetic construct; identical to SEQ ID NO: 145

SEQ ID NO: 328 - protein, 33 aa, synthetic construct
AEELKAAAELAKAGVSAEEVKEILKEAKRLGLG

SEQ ID NO: 329 - protein, 33 aa, synthetic construct
AEELKAAAELAKAGVSAEEIKELLKEAKELGLD

SEQ ID NO: 330 - protein, 33 aa, synthetic construct; identical to SEQ ID NO: 57

SEQ ID NO: 331 - protein, 33 aa, synthetic construct
AKDLEAAAKLAKAGVSAEEIKEILKEAKKLGLS

SEQ ID NO: 332 - protein, 33 aa, synthetic construct
AEALAAAAELAKAGVSAEEIAALLAQAKALGLG

SEQ ID NO: 333 - protein, 33 aa, synthetic construct
AEELKAAAELAKAGVSAKEIKELLKEAKELGLG

SEQ ID NO: 334 - protein, 33 aa, synthetic construct
AEALKAAAELAKAGVSAEEIAKILKLAKELGLS

SEQ ID NO: 335 - protein, 33 aa, synthetic construct
EEALKAAAELAKAGVSAEEIKKILKEAKKLGLG

SEQ ID NO: 336 - protein, 33 aa, synthetic construct
AEALKAAAELAKAGVSAEEVAKLLELAKKLGLG

SEQ ID NO: 337 - protein, 33 aa, synthetic construct
AEALKAAAELAKAGVSAEEIAKLLKLAKELGLD

SEQ ID NO: 338 - protein, 33 aa, synthetic construct
AEALKAAAELAKAGVSAEEVKKLLKEAKELGLD

SEQ ID NO: 339 - protein, 33 aa, synthetic construct; identical to SEQ ID NO: 191

SEQ ID NO: 340 - protein, 33 aa, synthetic construct; identical to SEQ ID NO: 119

SEQ ID NO: 341 - protein, 33 aa, synthetic construct
AEELKAAAELAKAGVSAEEVKKLLKEAKRLGLE

SEQ ID NO: 342 - protein, 33 aa, synthetic construct; identical to SEQ ID NO: 67

SEQ ID NO: 343 - protein, 33 aa, synthetic construct; identical to SEQ ID NO: 193

SEQ ID NO: 344 - protein, 33 aa, synthetic construct; identical to SEQ ID NO: 94

SEQ ID NO: 345 - protein, 33 aa, synthetic construct
AEALKAAAELAKAGVSAEEVKKILKEAKKLGLS

SEQ ID NO: 346 - protein, 33 aa, synthetic construct
SEALKAAAELAKAGVSAEEIKKILKEAKKLGLD

SEQ ID NO: 347 - protein, 33 aa, synthetic construct
AKALKAAAELAKAGVSAEEIAKLLKEAAKLGLD

SEQ ID NO: 348 - protein, 33 aa, synthetic construct
AEELKAAAKLAKAGISAKEIKELLKEAKELGLG

SEQ ID NO: 349 - protein, 33 aa, synthetic construct; identical to SEQ ID NO: 310

SEQ ID NO: 350 - protein, 33 aa, synthetic construct
AEELKAAAELAKAGVSAEEVKKLLKEAKALGLK

SEQ ID NO: 351 - protein, 33 aa, synthetic construct
AEALKAAAELAKAGVSAEEVKALLKEAKALGLG

SEQ ID NO: 352 - protein, 33 aa, synthetic construct
AEALKAAAELAKAGVSAKEIKELLKEAKELGLD

SEQ ID NO: 353 - protein, 33 aa, synthetic construct
EEALKAAAELAKAGVSAEEVLKLLKEAKELGLS

SEQ ID NO: 354 - protein, 33 aa, synthetic construct; identical to SEQ ID NO: 57

SEQ ID NO: 355 - protein, 33 aa, synthetic construct
EEALKAAAELAKAGVSAKEIKELLKEAKKLGLK

SEQ ID NO: 356 - protein, 33 aa, synthetic construct
AEALKAAAELAKAGVSAEEIKELLKEAAKLGLD

SEQ ID NO: 357 - protein, 33 aa, synthetic construct
EKALKAAAELAKAGVSAEEIKELLEKAKKLGLD

SEQ ID NO: 358 - protein, 33 aa, synthetic construct; identical to SEQ ID NO: 310

SEQ ID NO: 359 - protein, 33 aa, synthetic construct
AEALAAAAKLAEAGVSAEEIRELLREAARLGLG

SEQ ID NO: 360 - protein, 33 aa, synthetic construct
AEALKAAAELAKAGVSAEEVLKLLKEAKKLGLG

SEQ ID NO: 361 - protein, 33 aa, synthetic construct; identical to SEQ ID NO: 345

SEQ ID NO: 362 - protein, 33 aa, synthetic construct
AEELKAAAELAKAGVSAEEILKILKAAKELGLK

SEQ ID NO: 363 - protein, 33 aa, synthetic construct; identical to SEQ ID NO: 162

SEQ ID NO: 364 - protein, 33 aa, synthetic construct
AEALKAAAELAKAGVSAEEVKKLLKEAKKLGLS

SEQ ID NO: 365 - protein, 33 aa, synthetic construct; identical to SEQ ID NO: 190

SEQ ID NO: 366 - protein, 33 aa, synthetic construct
AEWIKAAAELAKAGVSAEEIKKILKEAKELGLP

SEQ ID NO: 367 - protein, 33 aa, synthetic construct
AEALKAAAELAKAGISAEEIKKLLKEAKKLGLG

SEQ ID NO: 368 - protein, 33 aa, synthetic construct
AEALKAAAELAKAGVSAEEIKKLLREAKELGLG

SEQ ID NO: 369 - protein, 33 aa, synthetic construct; identical to SEQ ID NO: 191

SEQ ID NO: 370 - protein, 33 aa, synthetic construct; identical to SEQ ID NO: 57

SEQ ID NO: 371 - protein, 33 aa, synthetic construct
AEALKAAAELAKAGVSAEEIAKLLELAKKLGLG

SEQ ID NO: 372 - protein, 33 aa, synthetic construct
AEALKAAAELAKAGVSAEEIKKLLKEAKELGLD

SEQ ID NO: 373 - protein, 33 aa, synthetic construct; identical to SEQ ID NO: 75

SEQ ID NO: 374 - protein, 33 aa, synthetic construct
AEALKAAAELAKAGVSAEEIMKILKEAKELGLG

SEQ ID NO: 375 - protein, 33 aa, synthetic construct
AEALKAAAELAKAGVSAEEVKELLREAKELGLG

SEQ ID NO: 376 - protein, 33 aa, synthetic construct; identical to SEQ ID NO: 313

SEQ ID NO: 377 - protein, 33 aa, synthetic construct; identical to SEQ ID NO: 57

SEQ ID NO: 378 - protein, 33 aa, synthetic construct
AEELKAAAELAKAGVSAEDIKKILKEAKELGLS

SEQ ID NO: 379 - protein, 33 aa, synthetic construct
AEALKAAAELAKAGVSAEEIAKLLREAKKLGLD

SEQ ID NO: 380 - protein, 33 aa, synthetic construct
EEELKAAAKLAKAGVSAEEIKKILKEAKKLGLK

SEQ ID NO: 381 - protein, 33 aa, synthetic construct
AEALKAAAELAKAGVSAEEIKELLKEAAKLGLG

SEQ ID NO: 382 - protein, 33 aa, synthetic construct
AKELEAAAKLAKAGVSAEEIKKLLKEAKKLGLS

SEQ ID NO: 383 - protein, 33 aa, synthetic construct
AEALKAAAELAKAGVSAKDIAALLKEAKKLGLD

SEQ ID NO: 384 - protein, 33 aa, synthetic construct
AEALKAAAELAKAGVSAEEVRKLLKEAKELGLS

SEQ ID NO: 385 - protein, 33 aa, synthetic construct
AEALKAAAELAKAGVSAEEIKKILKEAKKLGLT

SEQ ID NO: 386 - protein, 33 aa, synthetic construct
AEELKAAAELAKAGVSAEDIAKLLKEAKKLGLG

SEQ ID NO: 387 - protein, 33 aa, synthetic construct
AEWVKAAAELAKAGVSAEEIKEILREAKELGLD

SEQ ID NO: 388 - protein, 33 aa, synthetic construct
AEALKAAAELAKAGVSAEEIEKLLKEAKKLGLD

SEQ ID NO: 389 - protein, 33 aa, synthetic construct; identical to SEQ ID NO: 77

SEQ ID NO: 390 - protein, 33 aa, synthetic construct
AEALKAAAELAKAGVSPEEILKLLKEAKKLGLS

SEQ ID NO: 391 - protein, 33 aa, synthetic construct
AEALKAAAELAKAGVSAKEVKELLKEAKELGLD

SEQ ID NO: 392 - protein, 33 aa, synthetic construct; identical to SEQ ID NO: 153

SEQ ID NO: 393 - protein, 33 aa, synthetic construct; identical to SEQ ID NO: 196

SEQ ID NO: 394 - protein, 33 aa, synthetic construct
AEALKAAAELAKAGVSAEDIAKLLELAKKLGLG

SEQ ID NO: 395 - protein, 33 aa, synthetic construct
AEYLKAAAELAKAGVSAEEIAKLLKEAKKLGLG

SEQ ID NO: 396 - protein, 33 aa, synthetic construct
AEEIKAAAELAKAGVSAEEIKKILKEAKKLGLK

SEQ ID NO: 397 - protein, 33 aa, synthetic construct
AEALKAAAELAKAGVSAEEIKKLLKEAKELGLG

SEQ ID NO: 398 - protein, 33 aa, synthetic construct
AEALKAAAELAKAGVSAEEIKELLREAKRLGLD

SEQ ID NO: 399 - protein, 33 aa, synthetic construct
AEALAAAAKLAEAGVSAEEVKELLEEAARLGLP

SEQ ID NO: 400 - protein, 33 aa, synthetic construct
AEELKAAAELAKAGVSAEEVKEILKEAKKLGLE

SEQ ID NO: 401 - protein, 33 aa, synthetic construct
AEELKAAAELAKAGVSAEEIKELLKEAKELGLE

SEQ ID NO: 402 - protein, 33 aa, synthetic construct; identical to SEQ ID NO: 57

SEQ ID NO: 403 - protein, 33 aa, synthetic construct; identical to SEQ ID NO: 313

SEQ ID NO: 404 - protein, 33 aa, synthetic construct
EKELKAAAELAKAGVSAEEILKLLEKAKKLGLS

SEQ ID NO: 405 - protein, 33 aa, synthetic construct
AEALKAAAKLAKAGVSAKDIEKLLKEAKKLGLP

SEQ ID NO: 406 - protein, 33 aa, synthetic construct
AEELKAAAELAKAGVSAEEITKILKEAKALGLG

SEQ ID NO: 407 - protein, 33 aa, synthetic construct; identical to SEQ ID NO: 153

SEQ ID NO: 408 - protein, 33 aa, synthetic construct
AEELKAAAELAKAGVSAEDIMKILEKAKKLGLK

SEQ ID NO: 409 - protein, 33 aa, synthetic construct
AEAIKAAAKLAKAGVSAKEIKEILKEAKKLGLD

SEQ ID NO: 410 - protein, 33 aa, synthetic construct
AEALKAAAELAKAGVSAEEIKKLLEEAKKLGLD

SEQ ID NO: 411 - protein, 33 aa, synthetic construct
AEALKAAAKLAKAGVSAEEIKKILKEAAELGLG

SEQ ID NO: 412 - protein, 33 aa, synthetic construct
SEALKAAAKLAKAGVSAEEIKKLLKEAKRLGLS

SEQ ID NO: 413 - protein, 33 aa, synthetic construct
AEALEAAAKLAKAGVSAEEIAKLLREAKELGLG

SEQ ID NO: 414 - protein, 33 aa, synthetic construct
PEALKAAAELAKAGVSAEEIAKLLKEAKKLGLG

SEQ ID NO: 415 - protein, 33 aa, synthetic construct
AEALKAAAELAKAGVSAEEIKEILKEAKELGLD

SEQ ID NO: 416 - protein, 33 aa, synthetic construct
AEELKAAAELAKAGVSAEEVLELLREAKRLGLG

SEQ ID NO: 417 - protein, 33 aa, synthetic construct
AEELKAAAELAKAGVSAEEIKKLLKEAKKLGLE

SEQ ID NO: 418 - protein, 33 aa, synthetic construct; identical to SEQ ID NO: 67

SEQ ID NO: 419 - protein, 33 aa, synthetic construct; identical to SEQ ID NO: 210

SEQ ID NO: 420 - protein, 33 aa, synthetic construct
AEELKAAAELAKAGVSAKEIKEILKEAKKLGLG

SEQ ID NO: 421 - protein, 33 aa, synthetic construct
AEALKAAAELAKAGVSAEEVARLLREAKALGLG

SEQ ID NO: 422 - protein, 33 aa, synthetic construct
AEALKAAAELAKAGVSAEEIAKLLKEAKKLGLS

SEQ ID NO: 423 - protein, 33 aa, synthetic construct; identical to SEQ ID NO: 153

SEQ ID NO: 424 - protein, 33 aa, synthetic construct
AEELKAAAELAKAGVSAEEIKKILKEAKELGLD

SEQ ID NO: 425 - protein, 33 aa, synthetic construct
AEWLKAAAELAKAGVSAEEVKALLKEAKALGLP

SEQ ID NO: 426 - protein, 33 aa, synthetic construct
AEELKAAAELAKAGVSAEEIKKILKEAKELGLE

SEQ ID NO: 427 - protein, 33 aa, synthetic construct; identical to SEQ ID NO: 74

SEQ ID NO: 428 - protein, 33 aa, synthetic construct
AKEIKAAAELAKAGVSAEEIKKLLEEAKKLGLD

SEQ ID NO: 429 - protein, 33 aa, synthetic construct; identical to SEQ ID NO: 210

SEQ ID NO: 430 - protein, 33 aa, synthetic construct; identical to SEQ ID NO: 148

SEQ ID NO: 431 - protein, 33 aa, synthetic construct
AEALKAAAELAKAGVSAEEIERLLREAAELGLG

SEQ ID NO: 432 - protein, 33 aa, synthetic construct
AEELRAAAELAKAGVSAEDIARLLREAKRLGLG

SEQ ID NO: 433 - protein, 33 aa, synthetic construct
EEALKAAAELAKAGVSAEEIKKILKEAKELGLG

SEQ ID NO: 434 - protein, 33 aa, synthetic construct
AEEIKAAAKLAKAGVSAEEIKEILKEAKKLGLG

SEQ ID NO: 435 - protein, 33 aa, synthetic construct
AKELEAAAKLAKAGVSAEEIRKLLKEAKKLGLE

SEQ ID NO: 436 - protein, 33 aa, synthetic construct; identical to SEQ ID NO: 239

SEQ ID NO: 437 - protein, 33 aa, synthetic construct; identical to SEQ ID NO: 173

SEQ ID NO: 438 - protein, 33 aa, synthetic construct
AEELKAAAELAKAGVSAEEIKELLKEAKKLGLS

SEQ ID NO: 439 - protein, 33 aa, synthetic construct
AEELKAAAELAKAGISAKEIKELLELAKKLGLQ

SEQ ID NO: 440 - protein, 33 aa, synthetic construct
AEELKAAAELAKAGVSAEEVKKLLKEAKELGLG

SEQ ID NO: 441 - protein, 33 aa, synthetic construct; identical to SEQ ID NO: 177

SEQ ID NO: 442 - protein, 33 aa, synthetic construct
AEALKAAAELAKAGVSAEEVKEILELAKKLGLG

SEQ ID NO: 443 - protein, 33 aa, synthetic construct; identical to SEQ ID NO: 57

SEQ ID NO: 444 - protein, 33 aa, synthetic construct
AEELKAAAELAKAGVSAEEIERLLREAKELGLE

SEQ ID NO: 445 - protein, 33 aa, synthetic construct
AEAIKAAAELAKAGVSAEDIQKILKEAKKLGLP

SEQ ID NO: 446 - protein, 33 aa, synthetic construct
AKALEAAAKLAKAGVSAEEIKKLLKEAKELGLD

SEQ ID NO: 447 - protein, 33 aa, synthetic construct; identical to SEQ ID NO: 190

SEQ ID NO: 448 - protein, 33 aa, synthetic construct
AEELKAAAELAKAGVSAEEIAALLREAKALGLD

SEQ ID NO: 449 - protein, 33 aa, synthetic construct
AEALKAAAKLAAAGVSAEDIAALLREAAALGLS

SEQ ID NO: 450 - protein, 33 aa, synthetic construct
AEALKAAAKLAKAGVSAEEIKKLLKEAKKLGLD

SEQ ID NO: 451 - protein, 33 aa, synthetic construct
AEALKAAAKLAKAGVSAEEVERLLKEAKELGLD

SEQ ID NO: 452 - protein, 33 aa, synthetic construct
AEELKAAAELAKAGVSAEEIAKLLKLAKALGLG

SEQ ID NO: 453 - protein, 33 aa, synthetic construct
AEALEAAAKLAKAGVSAEEVKELLKEAKELGLG

SEQ ID NO: 454 - protein, 33 aa, synthetic construct
AEALKAAAELAKAGVSAEEIKKLLQEAKALGLG

SEQ ID NO: 455 - protein, 33 aa, synthetic construct
AEELKAAAKLAKAGVSAKEVKEILKEAKKLGLK

SEQ ID NO: 456 - protein, 33 aa, synthetic construct
EEYLKAAAKLAKAGVSAEEIKKILKEAKKLGLK

SEQ ID NO: 457 - protein, 33 aa, synthetic construct
AEALAAAAELAKAGVSAADIAALLREAAALGLD

SEQ ID NO: 458 - protein, 33 aa, synthetic construct
AEALKAAAKLAKAGVSAEEIKEILKEAKRLGLD

SEQ ID NO: 459 - protein, 33 aa, synthetic construct
AEALAAAAELAKAGVSAEEIAALLREAAALGLG

SEQ ID NO: 460 - protein, 33 aa, synthetic construct
AEELKAAAELAKAGVSAEEIAKLLELAKRLGLE

SEQ ID NO: 461 - protein, 33 aa, synthetic construct
AEALKAAAELAKAGVSAEEVKELLKLAKELGLD

SEQ ID NO: 462 - protein, 33 aa, synthetic construct
AEELKAAAELAKAGVSAKDITAILKEAKKLGLG

SEQ ID NO: 463 - protein, 33 aa, synthetic construct
AEEIKAAAELAKAGVSAEEIKEILKEAKKLGLG

SEQ ID NO: 464 - protein, 33 aa, synthetic construct
AEALKAAAELAKAGVSAEEIEKLLEKAKKLGLD

SEQ ID NO: 465 - protein, 33 aa, synthetic construct
AEALKAAAELAKAGVSAKEVKELLKEAKKLGLD

SEQ ID NO: 466 - protein, 33 aa, synthetic construct
AEELKAAAELAKAGVSAEDIKKLLKEAKKLGLE

SEQ ID NO: 467 - protein, 33 aa, synthetic construct
AEALKAAAELAKAGVSAEEIAALLKEAAALGLD

SEQ ID NO: 468 - protein, 33 aa, synthetic construct
AEALKAAAELAKAGVSAKEIAALLREAKALGLG

SEQ ID NO: 469 - protein, 33 aa, synthetic construct; identical to SEQ ID NO: 153

SEQ ID NO: 470 - protein, 33 aa, synthetic construct
AEALKAAAELAKAGVSAEEIAKLLREAAALGLG

SEQ ID NO: 471 - protein, 33 aa, synthetic construct
AEALKAAAKLAEAGVSAEDIAKLLKEAKKLGLG

SEQ ID NO: 472 - protein, 33 aa, synthetic construct
AEALKAAAELAKAGVSAKEIKELLKEAKKLGLG

SEQ ID NO: 473 - protein, 33 aa, synthetic construct
EEMLKAAAELAKAGVSAEEIKELLKEAKKLGLG

SEQ ID NO: 474 - protein, 33 aa, synthetic construct
AEWLEAAAKLAEAGVSAEEVAKLLKEAKKLGLG

SEQ ID NO: 475 - protein, 33 aa, synthetic construct
AEELKAAAKLAKAGVSAEDIKKILKEAKELGLK

SEQ ID NO: 476 - protein, 33 aa, synthetic construct
AEELKAAAELAKAGVSAEEIKEILKLAKELGLG

SEQ ID NO: 477 - protein, 33 aa, synthetic construct; identical to SEQ ID NO: 153

SEQ ID NO: 478 - protein, 33 aa, synthetic construct
AKALEAAAKLAKAGVSAEEIKKLLKEAKKLGLD

SEQ ID NO: 479 - protein, 33 aa, synthetic construct
AEWLKAAAELAKAGVSAEEVKKILKEAKKLGLG

SEQ ID NO: 480 - protein, 33 aa, synthetic construct
AEELKAAAELAKAGVSAEEIKKILKEAKELGLG

SEQ ID NO: 481 - protein, 33 aa, synthetic construct
EKALKAAAELAKAGISAEEIKKILKEAKKLGLG

SEQ ID NO: 482 - protein, 33 aa, synthetic construct
AEELKAAAELAKAGVSAEEVKELLKEAKKLGLS

SEQ ID NO: 483 - protein, 33 aa, synthetic construct
AEALEAAAKLAEAGVSAEEIAALLKEAARLGLG

SEQ ID NO: 484 - protein, 33 aa, synthetic construct; identical to SEQ ID NO: 90

SEQ ID NO: 485 - protein, 33 aa, synthetic construct
AKMVKEAAKLAKAGVSAEEIKKIMKEAKKLGLD

SEQ ID NO: 486 - protein, 33 aa, synthetic construct
AEALKAAAELAKAGISAEEIKELLKEAKKLGLG

SEQ ID NO: 487 - protein, 33 aa, synthetic construct
AEALKAAAKLAKAGVSAEEIKKLLKEAKELGLG

SEQ ID NO: 488 - protein, 33 aa, synthetic construct
AEALKAAAELAKAGVSAEEIAALLRLAAELGLG

SEQ ID NO: 489 - protein, 33 aa, synthetic construct
AEALKAAAELAKAGVSAEEIKELLKEAKKLGLK

SEQ ID NO: 490 - protein, 33 aa, synthetic construct; identical to SEQ ID NO: 133

SEQ ID NO: 491 - protein, 33 aa, synthetic construct
AEALKAAAKLAEAGVSAEEIKELLKEAKELGLD

SEQ ID NO: 492 - protein, 33 aa, synthetic construct
AEWLKAAAELAKAGVSAKEIKELLKEAKKLGLG

SEQ ID NO: 493 - protein, 33 aa, synthetic construct
AEALKAAAELAKAGVSAEEIERILKEAKKLGLG

SEQ ID NO: 494 - protein, 33 aa, synthetic construct
AEALKAAAELAKAGVSAEEVKEILKEAKELGLD

SEQ ID NO: 495 - protein, 33 aa, synthetic construct; identical to SEQ ID NO: 486

SEQ ID NO: 496 - protein, 33 aa, synthetic construct
AKELEAAAKLAKAGVSAEEILKILREAKKLGLG

SEQ ID NO: 497 - protein, 33 aa, synthetic construct
AEELKAAAKLAKAGVSAEEIKKILEEAKKLGLG

SEQ ID NO: 498 - protein, 33 aa, synthetic construct
AEALKAAAKLAKAGVSAKEIKELLKEAKALGLD

SEQ ID NO: 499 - protein, 33 aa, synthetic construct
AEALKAAAELAKAGVSAEDIAKLLKEAKKLGLG

SEQ ID NO: 500 - protein, 33 aa, synthetic construct; identical to SEQ ID NO: 396

SEQ ID NO: 501 - protein, 33 aa, synthetic construct
SEALKAAAELAKAGISAEEIKKILKEAKELGLG

SEQ ID NO: 502 - protein, 33 aa, synthetic construct; identical to SEQ ID NO: 417

SEQ ID NO: 503 - protein, 33 aa, synthetic construct
AEELKAAAELAKAGVSAEEIKELLKEAKKLGLG

SEQ ID NO: 504 - protein, 33 aa, synthetic construct; identical to SEQ ID NO: 422

SEQ ID NO: 505 - protein, 33 aa, synthetic construct
EKALEAAAKLAKAGVSAEEIIKILKEAKKLGLG

SEQ ID NO: 506 - protein, 33 aa, synthetic construct
EEELKAAAELAKAGVSAEDIKKILKEAKELGLK

SEQ ID NO: 507 - protein, 33 aa, synthetic construct
AEALKAAAELAKAGVSAKEVKEILKEAKKLGLG

SEQ ID NO: 508 - protein, 33 aa, synthetic construct
EEALKAAAELAKAGVSAEEVKKLLREAKELGLD

SEQ ID NO: 509 - protein, 33 aa, synthetic construct
AEALAAAAKLAEAGVSAEEIAALLKLAAELGLG

SEQ ID NO: 510 - protein, 33 aa, synthetic construct
AEELKAAAELAKAGVSAEEIAKLLKLAKELGLD

SEQ ID NO: 511 - protein, 33 aa, synthetic construct
AEALKAAAELAKAGVSAEDVERLLREAKRLGLG

SEQ ID NO: 512 - protein, 33 aa, synthetic construct; identical to SEQ ID NO: 450

SEQ ID NO: 513 - protein, 33 aa, synthetic construct
AEALKAAAELAKAGVSAEEIAKLLKLAAKLGLG

SEQ ID NO: 514 - protein, 33 aa, synthetic construct
AEALKAAAKLAKAGVSAEEIKKLLREAKKLGLG

SEQ ID NO: 515 - protein, 33 aa, synthetic construct
AEALKAAAELAKAGVSAEEIKKLLKEAAELGLG

SEQ ID NO: 516 - protein, 33 aa, synthetic construct
AEALKAAAELAKAGVSAEEVAALLKEAKALGLG

SEQ ID NO: 517 - protein, 33 aa, synthetic construct
AEAIKAAAELAKAGVSAEEIKKILKEAKELGLD

SEQ ID NO: 518 - protein, 33 aa, synthetic construct
AESLAAAAKLAKAGVSAEEIKKLLEEAKKLGLK

SEQ ID NO: 519 - protein, 33 aa, synthetic construct
AESLKAAAELAKAGVSAEEIKKLLKEAKKLGLG

SEQ ID NO: 520 - protein, 33 aa, synthetic construct
AEALKAAAELAKAGVSAEDIERLLREAARLGLS

SEQ ID NO: 521 - protein, 33 aa, synthetic construct
AEALKAAAELAKAGVSAEEIEKILKTAKELGLD

SEQ ID NO: 522 - protein, 33 aa, synthetic construct
AEALKAAAKLAKAGVSAKEIAALLKEAKKLGLG

SEQ ID NO: 523 - protein, 33 aa, synthetic construct; identical to SEQ ID NO: 480

SEQ ID NO: 524 - protein, 33 aa, synthetic construct
AEALKAAAELAKAGVSAEEIKEILKEAKKLGLG

SEQ ID NO: 525 - protein, 33 aa, synthetic construct
AEALKAAAKLAKAGVSAEEVKELLKEAAKLGLG

SEQ ID NO: 526 - protein, 33 aa, synthetic construct
AEALKAAAELAKAGVSAEEIAKLLKEAAKLGLG

SEQ ID NO: 527 - protein, 33 aa, synthetic construct
SEALKAAAKLAKAGVSAEEIKKLLKEAKKLGLE

SEQ ID NO: 528 - protein, 33 aa, synthetic construct
AEELKAAAELAKAGVSAKEIKEILKEAKKLGLK

SEQ ID NO: 529 - protein, 33 aa, synthetic construct
AEYIKAAAELAKAGVSAEDIEKILKEAKKLGLD

SEQ ID NO: 530 - protein, 33 aa, synthetic construct; identical to SEQ ID NO: 193

SEQ ID NO: 531 - protein, 33 aa, synthetic construct
AEELKAAAKLAKAGISAEEIAKLLKLAKKLGLG

SEQ ID NO: 532 - protein, 33 aa, synthetic construct
AEELKAAAELAKAGVSAEEVKELLREAKKLGLE

SEQ ID NO: 533 - protein, 33 aa, synthetic construct
AEALKAAAELAKAGVSAEEVAAILREAKALGLD

SEQ ID NO: 534 - protein, 33 aa, synthetic construct
AEALKAAAELAKAGISAEEIAKLLKLAKELGLG

SEQ ID NO: 535 - protein, 33 aa, synthetic construct
AEELKAAAELAKAGISAEEIKKILKEAKELGLK

SEQ ID NO: 536 - protein, 33 aa, synthetic construct; identical to SEQ ID NO: 67

SEQ ID NO: 537 - protein, 33 aa, synthetic construct
AKWLEAAAKLAKAGVSAEEIAKLLKEAKALGLE

SEQ ID NO: 538 - protein, 33 aa, synthetic construct
AEELKAAAELAKAGVSAKDIAKLLKEAKELGLS

SEQ ID NO: 539 - protein, 33 aa, synthetic construct
AEALKAAAELAKAGVSAEDIKKLLKEAKKLGLG

SEQ ID NO: 540 - protein, 33 aa, synthetic construct
AEELKAAAELAKAGVSAEEIKKILREAKELGLE

SEQ ID NO: 541 - protein, 33 aa, synthetic construct
AEALKAAAELAKAGVSAKEIAALLKEAAALGLG

SEQ ID NO: 542 - protein, 33 aa, synthetic construct; identical to SEQ ID NO: 94

SEQ ID NO: 543 - protein, 33 aa, synthetic construct; identical to SEQ ID NO: 319

SEQ ID NO: 544 - protein, 33 aa, synthetic construct; identical to SEQ ID NO: 191

SEQ ID NO: 545 - protein, 33 aa, synthetic construct; identical to SEQ ID NO: 310

SEQ ID NO: 546 - protein, 33 aa, synthetic construct
AEAIKAAAELAKAGVSPEEIKKILKEAKKLGLG

SEQ ID NO: 547 - protein, 33 aa, synthetic construct
AEAVKAAAELAKAGVSAEDIKKILKEAAKLGLD

SEQ ID NO: 548 - protein, 33 aa, synthetic construct
AEALKAAAELAKAGVSAEEIKELLREAKKLGLG

SEQ ID NO: 549 - protein, 33 aa, synthetic construct
AEELKAAAELAKAGVSAEDIKKILKEAKKLGLE

SEQ ID NO: 550 - protein, 33 aa, synthetic construct
AEELKAAAKLAKAGVSAEEIKKLLELAKKLGLG

SEQ ID NO: 551 - protein, 33 aa, synthetic construct
AEALKAAAELAKAGVSAEEIKKLLKEAAKLGLD

SEQ ID NO: 552 - protein, 33 aa, synthetic construct
EEYLKAAAELAKAGVSAEEIKEILEKAKKLGLK

SEQ ID NO: 553 - protein, 33 aa, synthetic construct; identical to SEQ ID NO: 313